SEQUENCE LISTING (65 sequences)

SEQ ID NO: 1 - DNA, 195 bp, Homo sapiens
gctgctaaaagccgggcccgcggcgcagcgcacggagccctctgccgcccgcgcggagaaggcgccgggcgcggtccggcggtcccaagcatccccgcagcgcccccgccgcccgccgcccgcccctggccgcgccgcgcccctctgcaggtcgagcggattccaggtgagggcgtggagcccccgcgtcatcag

SEQ ID NO: 2 - protein, 675 aa, Homo sapiens
MREYHKWRIYYKKRLRKPSREDDLLAPKQAEGRWPPPEQWCKQLFSSVVPVLLGDPEEEPGGRQLLDLNCFLSDISDTLFTMTQSGPSPLQLPPEDAYVGNADMIQPDLTPLQPSLDDFMDISDFFTNSRLPQPPMPSNFPEPPSFSPVVDSLFSSGTLGPEVPPASSAMTHLSGHSRLQARNSCPGPLDSSAFLSSDFLLPEDPKPRLPPPPVPPPLLHYPPPAKVPGLEPCPPPPFPPMAPPTALLQEEPLFSPRFPFPTVPPAPGVSPLPAPAAFPPTPQSVPSPAPTPFPIELLPLGYSEPAFGPCFSMPRGKPPAPSPRGQKASPPTLAPATASPPTTAGSNNPCLTQLLTAAKPEQALEPPLVSSTLLRSPGSPQETVPEFPCTFLPPTPAPTPPRPPPGPATLAPSRPLLVPKAERLSPPAPSGSERRLSGDLSSMPGPGTLSVRVSPPQPILSRGRPDSNKTENRRITHISAEQKRRFNIKLGFDTLHGLVSTLSAQPSLKVSKATTLQKTAEYILMLQQERAGLQEEAQQLRDEIEELNAAINLCQQQLPATGVPITHQRFDQMRDMFDDYVRTRTLHNWKFWVFSILIRPLFESFNGMVSTASVHTLRQTSLAWLDQYCSLPALRPTVLNSLRQLGTSTSILTDPGRIPEQATRAVTEGTLGKPL

SEQ ID NO: 3 - DNA, 1195 bp, Homo sapiens
agagggagagaggagcctggaggagagaagagagagggagaggaaagggagggggagagagagaaagagagaaagcggggagagaagagagtaagagtgagggatgagagtgagagagagtgctggggggactcagggactaggaggaggttcaaagccgggctggggctctgggaggagcccgaggcggtcccagcgtctttctctgcccacctggggtggccccacacctgcggctcctccctccccgagcccagcccgcggctgggggccgggcaccgagcccccggggaaagtagagggggttaaaggtgagcgggcacgagccggcggcagacggggattggcaggcgcccgagcccagggcccgggccatggctgccccacaggcctctgagcggctgagcggctgccgcccacgtgctaaggagagaaggaggcaccagatcaaggggcaaaggacacctcggagcggggcgggggtgcccgcgtccacacccgctgctaaaagccgggcccgcggcgcagcgcacggagccctctgccgcccgcgcggagaaggcgccgggcgcggtccggcggtcccaagcatccccgcagcgcccccgccgcccgccgcccgcccctggccgcgccgcgcccctctgcaggtcgagcggattccaggtgagggcgtggagcccccgcgtcatcaggtgggtgcaggggcctcacccaaggggaattggatccgcctctgtgctgggcctcagtttcccctctgcccatagaggcgcgcgtctctggctcagctgggttgaaggggttcataggcttatgtggcctcctcgctcagcctcagctgccaggaggaaggaggcaaactcgtaccccagggaggactccggatcaggaatccaagaattcatcctgctagaaggtgaagtgcacacgggtcctggaagccccgtgtctcccactggctggactgggctgggcttccatcctggtctgggaggttctcccaggagctgagagggggcccagggagtcaggagggagggccttcctccctctggctctccctgccctgggcaggtgacatgccagtggagaaaccctgtccaggtgggccctgggatgaggaggaccaaaactgcccctggctgggacagaaagaagggagatggcagggtggggtatgtggattctggcc

SEQ ID NO: 4 - DNA, 3132 bp, Homo sapiens
gctgctaaaagccgggcccgcggcgcagcgcacggagccctctgccgcccgcgcggagaaggcgccgggcgcggtccggcggtcccaagcatccccgcagcgcccccgccgcccgccgcccgcccctggccgcgccgcgcccctctgcaggtcgagcggattccaggtgagggcgtggagcccccgcgtcatcagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactcagtgcccagcccagcctcaaggtgagcaaagctaccacgctgcagaagacagctgagtacatccttatgctacagcaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttctctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtctgctggtt

SEQ ID NO: 5 - DNA, 171 bp, Mus musculus
agtctaaaagccgggcttcgtgcgggcgcagcgcacggagccctctgccgcctgcgcggggaaggcgcggggtgaggccgcagcggcctgagcatctgcagcctcgcggagacccgaggtcccaggatccgagcccagcccgacgccatctgcagatcgcgtggagctcag

SEQ ID NO: 6 - protein, 687 aa, Mus musculus
MREYHKWRIYYKKRLRKSSREGDFLAPKQVEGGWPPPERWCEQLFSSVVPVLLGGSEEEPGGRQLLDLDCFLSDISDTLFTMTQPSPSSLQLPPEDAYVGNADMIQPDLTPLQPSLDDFMEISDFFTNYRPPQTPTSSNYIESPSFGPMADSLFSSGILAPEMPSPASSSSSSGMTPHSGNTRLQARNSCSGPLDPNPFLSSEFLLPEDPKTKIPPAPGPTPLLPFPTPVKVHGLEPCTPSPFPTMAPPPSLLPEESLLSARFPFTSAPPAPGVSTLPAPTTFVPTPQPGPGPVPFSVDHLPHGYLEPVFGPHFTVPQGMQPRCKPSSPSPGGQKASPPTLASATASPTATATARDNNPCLTQLLRAAKPEQALEPPTMPGTLLRPPESPQDTVSEIPRARAFFPPIPAPTPPRPPPGPATLAPPRSLVVPKAERLSPPASSGSERRLSGDLNSIQPSGALSVHLSPPQTVLSRGRVDNNKMENRRITHISAEQKRRFNIKLGFDTLHGLVSTLSAQPSLKVSKATTLQKTAEYILMLQQERAAMQEEAQQLRDEIEELNAAINLCQQQLPATGVPITHQRFDQMRDMFDDYVRTRTLHNWKFWVFSILIRPLFESFNGMVSTASLHSLRQTSLAWLEQYCSLPALRPTVLNSLRQLSTSTSILTDPSLVPEQATRAVTEGTLGRPL

SEQ ID NO: 7 - DNA, 1171 bp, Mus musculus
tggctagggagatgtggcgtttggggaagagaagagaagcttagggaacaaggcctcctcaggggaggactcagggtagagcagactccctgggagatagatggagaccagtgagttcatggaagctgcagacaagggaggcccacaaccccgagtgagtttcaaataggggctggggctggggctgagactccttctactctctcggtcccaaatctggggctcctcccttctcgcacccggtctacagtttgggacctagacatcaagcccccggggagaggggggttaaaggtgagcggggcacgagcggggcggcagaaggtgattggcaggctcctgagcccacggcgcgcgccaccgccgctccacaggccaccagcctgaggagcggctgccatccacgtgctaaggagagaaggaggcaccggaccaaggggcaaaggacacctcggagcgggagggcgggggaacctgcagcgcccagcagtccacaccccagtctaaaagccgggcttcgtgcgggcgcagcgcacggagccctctgccgcctgcgcggggaaggcgcggggtgaggccgcagcggcctgagcatctgcagcctcgcggagacccgaggtcccaggatccgagcccagcccgacgccatctgcagatcgcgtggagctcaggtgagcaggaaagatcttcagttatcagagggacatagatgcctcgcagaggaggactcagagccctgccagacctcagtttccagtgtgcaatgaagaggggtgttcctaaatcacgactggagtggggggctcccactctctgtgcctctcagcccccacttgcacatgctcagagccagtggggaaagagaccgactcaggctccagaaaggcttggacatcaggaatgctagtctatcttgtttgaccccttaggatcacagggtcctgtttcctcctgggtcccccatgctgagctgggactgaggggtgatctggtgaaggggggcctgcctggatccagggaggcaggagggaggggacagatgccagggtggagcctgtggatttccggtcacccagcctggcagcaggcaggggctagggacttagttgaaggcctacccactcggtgtagcctaaaagagtcagagctttgtgttggccctcagccatac

SEQ ID NO: 8 - DNA, 3035 bp, Mus musculus
agtctaaaagccgggcttcgtgcgggcgcagcgcacggagccctctgccgcctgcgcggggaaggcgcggggtgaggccgcagcggcctgagcatctgcagcctcgcggagacccgaggtcccaggatccgagcccagcccgacgccatctgcagatcgcgtggagctcagtggcaagctggtctctcccaagtggaagaacttcaaaggcctcaagttgctatgccgggacaagatccggctgaacaacgccatctggagagcctggtacattcagtatgtgcaacggaggaagagcccagtgtgtggtttcgtgacccctctgcaggggtctgaagcagatgagcaccggaaacctgaggctgtcatcctggagggtaattactggaagcggcgcatcgaggtggtgatgcgcgaataccacaagtggagaatctactacaaaaagcggctccgtaagtccagcagggaaggggatttcctggctcccaagcaggtggaaggtgggtggccgccaccagagcgatggtgcgaacagctcttctccagcgtggtgcctgtgctgcttgggggctccgaggaggagcctgggggccggcagcttctagacctggactgcttcttgtccgatatctccgacacactcttcaccatgacacagcccagtccttcgtccctgcagctgcccccagaagatgcttatgttggcaatgctgacatgatccagccagacctgacgccgctgcagcccagcctagatgacttcatggagatatcagatttcttcaccaattaccgcccccctcagacacccacatcttcaaactacatagagtcccccagctttggccccatggctgactccctcttcagcagtgggatcctggccccagagatgccatctccagcctcgtcttcctcctcctcagggatgacccctcactcagggaatacacgcctacaggctcggaacagctgctctggacccttggatcccaacccctttctgagctctgaattccttcttcctgaagaccctaagaccaagatcccacctgctcctggaccgacccctctccttccattccccacccctgtcaaggtgcatggcttagagccctgcaccccctctcccttccccacaatggctccgcccccctctttgttgccagaagaatctctcctctctgccaggttcccattcacctcagcccctcctgctccaggagtgtctacgcttcctgcacccacaacctttgtccccaccccacagcccggccctggtcccgtccccttctctgtagaccatctgccccatgggtacctggaacccgtctttgggcctcacttcactgtgcctcagggtatgcaacccaggtgcaagccctcctccccatcccctgggggacaaaaagcaagtccacccaccttggcctctgccaccgccagtcccactgccactgccactgccagagacaacaacccctgtcttacacagctgctcagggcagccaagcctgagcaagcactggagcctccgactatgcccggtaccctcctccggcctccagagtccccgcaggatacagtttctgaaatcccccgtgcccgtgcctttttccccccaatcccggcccctacaccaccccggccacctccaggcccagccacattggctcctcccaggtccctggttgtccccaaagcagagcggctctcgcctccagcctccagcggcagtgagcggcgactatcaggggatctcaactccatacaaccctcgggggcactgagtgtccacctgtctccccctcaaactgtcctaagccggggtcgtgtagacaacaacaagatggagaaccgacgtatcacacacatctccgcggagcagaagaggcggttcaatattaagctaggattcgacaccctgcacggacttgtcagcacgctcagtgcccagcccagcctcaaggtgagcaaagcaaccacgcttcagaagacagcggagtacatcctgatgctgcagcaggaacgggcagctatgcaggaggaggcgcagcagctgcgggatgaaatagaggagctcaacgctgccatcaacttgtgccagcagcagctaccggccaccggggtgcccatcacacatcagcgctttgaccagatgcgggacatgtttgatgactatgtccggacgcgcacgctgcacaactggaagttctgggtgttcagcatcctcatccgacctttatttgaatccttcaatgggatggtgtctaccgccagcttgcacagcctccgccagacctcactggcctggctggaacagtactgttccctacctgctctccggccaactgtcctgaattcccttcgccaactcagcacttccacaagcatcctgactgaccccagccttgtgcctgagcaagccacacgggcagtcacagagggcaccctggggagaccattataatcctggtgaaagtttccaagctccaggaacaacttgtgaacacgcttctaaaaatcctggctggccgtttccctgcagatctggaaggttccaagtgttgcactcaccacctccttggggatggagaaaaacctagttccattcctgcactgttgctaatgctatgatccctggtgactcatatcccatctttggacctccctttaataacagagggccccaaatgcattagcagctgcagcactggggacctaaacaggagctctgatgagagatgtcggtctgtttcctcacaacttcctgctttggataaagggtaccatccttagttcctgagcagagaggcagagcaggagatctcctctctttgcctttgcactagtgacatggggagctatagggtggcttcctgactgaggccactggggtctgatgccagaagagatcagggcaagggctagccagagcatggcaggcatcttgcatgaatgtagatgtgtgtgtgcgtatgtggattttataaaaaagaaaatgttcttcatcaataaaagcaaaaactgtc

SEQ ID NO: 9 - DNA, 12 bp, Mus musculus
atccacgtgcta

SEQ ID NO: 10 - DNA, 12 bp, Homo sapiens
gcccacgtgcta

SEQ ID NO: 11 - DNA, 12 bp, Pongo abelii
gcccacgtgcta

SEQ ID NO: 12 - DNA, 12 bp, Canis lupus familiaris
acccacgtgcta

SEQ ID NO: 13 - DNA, 12 bp, Equus ferus caballus
acccacgtgcta

SEQ ID NO: 14 - DNA, 12 bp, Monodelphis domestica
tcccacgtgcta

SEQ ID NO: 15 - DNA, 27 bp, Mus musculus
tcgcgtggagctcaggtgagcaggaaa

SEQ ID NO: 16 - DNA, 27 bp, Homo sapiens
tcgagcggattccaggtgaggcgtgga

SEQ ID NO: 17 - DNA, 27 bp, Pongo abelii
tcgagcggattccaggtgaggcgtgga

SEQ ID NO: 18 - DNA, 27 bp, Canis lupus familiaris
tagtgaggaatccaggtgaggcgtgga

SEQ ID NO: 19 - DNA, 27 bp, Equus ferus caballus
ccgcgcagaatccaggtgaggcgtgga

SEQ ID NO: 20 - DNA, 27 bp, Monodelphis domestica
tcccaaggaaaccaggtgaggggacga

SEQ ID NO: 21 - DNA, 345 bp, Artificial Sequence
  Consensus Sequence
ctgagcggctgccacccacgtgctaaggagagaaggaggcaccggatcaaggggcaaaggacacctcggagcgggngngcgggggtgcccgcgtccacacccgctgctaaaagccgggcccgnggcgcagcgcacggagccctctgccgcccgcgcngagaagncgcggggcgcggccgccnnggcccgagcatcccngcagcnnccccgcggcccgccncccgncgnnnnnngncctggccgcgncccgccccanctgcagatcgcgcggaatccaggtgagggggntggngcnccgngncatcaggtgggtgcaggtgcctcncccanggggantngganccc

SEQ ID NO: 22 - DNA, 351 bp, Mus musculus
aggagcggctgccatccacgtgctaaggagagaaggaggcaccggaccaaggggcaaaggacacctcggagcgggagggcgggggaacctgcagcgcccagcagtccacaccccagtctaaaagccgggcttcgtgcgggcgcagcgcacggagccctctgccgcctgcgcggggaaggcgcggggtgaggccgcagcggcctgagcatctgcagcctcgcggagacccgaggtcccaggatccgagcccagcccgacgccatctgcagatcgcgtggagctcaggtgagcaggaaagatcttcagttatcagagggacatagatgcctcgcagaggaggactcagagccc

SEQ ID NO: 23 - DNA, 332 bp, Homo sapiens
ctgagcggctgccgcccacgtgctaaggagagaaggaggcaccagatcaaggggcaaaggacacctcggagcggggcgggggtgcccgcgtccacacccgctgctaaaagccgggcccgcggcgcagcgcacggagccctctgccgcccgcgcggagaaggcgccgggcgcggtccggcggtcccaagcatccccgcagcgcccccgccgcccgccgcccgcccctggccgcgccgcgcccctctgcaggtcgagcggattccaggtgagggcgtggagcccccgcgtcatcaggtgggtgcaggggcctcacccaaggggaattggatccg

SEQ ID NO: 24 - DNA, 350 bp, Pongo abelii
ctgagcggctgccgcccacgtgctaaggagggaaggaggcaccggatcaaggggcaaaggacatctcggagcggggcgggggtgcccgcgtccacacccgctgctaaaagccgggtccgcggcgcagcgcacggagccctctgccgcccgtgcggagaaggcgccgagcgcggtcgggcggtcccaagcatccccgcaccgcccccgccgcccgccgcccgccgcccgcgggacccccggccctggccgcgccgcgcccctctgcaggtcgagcggattccaggtgagggcgtggagcccccgcgtcatcaggtgggtgcaggggcctcactcaaggggaattggatccg

SEQ ID NO: 25 - DNA, 357 bp, Canis lupus familiaris
gtgagcggctgccacccacgtgctaaggagagaaggaggcaccggatcaaggggcaaaggacatctggagcaggcgggggccgggcagtgcccagtccacacccgccactaaaagccgggcccagggtgcagtgcatagagccctctgccgccagcgcagagaagacgcggccgctgtcggcctcggcccgggagcatcctgcatgtgccccgtggcccggaccccagacaccgctggaggggactctggggcgctgcctgggacctgctacaccagcagatagtgaggaatccaggtgaggggcgtggagggcatcaggtgggtgcaggtgcctaggccagggggagctgatcctc

SEQ ID NO: 26 - DNA, 356 bp, Equus ferus caballus
gtgagcggctgccacccacgtgctaaggagagacggaggcaccggatcaaggggcaaaggacacccctagcgagcggcgggcgggcgggcgggggccgggtagtgcccgcgtccacacccgctgctaaaagctgtccgcgggcgcagcgcagagcacggagcgctctgccgccggtgcagaaaagacgtgggagcctccgcggccgagcatcccgcgagcacctcgcggcccggccccggacgccgtctgggacccgccacacccgcggaccgcgcagaatccaggtgaggggcgtggagggcatcggccccaggggagtggtagtctcctctccgtatcctggacctcagcctcc

SEQ ID NO: 27 - DNA, 366 bp, Monodelphis domestica
ctgatcggctgcctcccacgtgctaaggagagaaggaggcaccagagagagggtcaaaggacaccttgaagagaccagtgataggcagcctggggacagatcatagggaacgactataaggctctgagtctcaagcagaacagcccacagagccctctggtgcccgagcagagcagagtgtgacccgggcagcctgggcagggaggagggacagagcatccccaagccacccgctggggctgccccaggagggggacgtgaaggagcagccactcacgccacccaccgctcccaaggaaaccaggtgagggatcttcgggttagggaaggggacgaggacgggtgacagccaaggaaggaggaggc

SEQ ID NO: 28 - DNA, 20 bp, Mus musculus
ttgactcaacacgggaaacc

SEQ ID NO: 29 - DNA, 20 bp, Mus musculus
agacaaatcgctccaccaac

SEQ ID NO: 30 - DNA, 23 bp, Mus musculus
tgtacaagcagtgtgggctggct

SEQ ID NO: 31 - DNA, 22 bp, Mus musculus
ccacatggcctggcttggaggg

SEQ ID NO: 32 - DNA, 17 bp, Mus musculus
gccagcgggagcacatc

SEQ ID NO: 33 - DNA, 21 bp, Mus musculus
ctttgcaggtgccacttcatc

SEQ ID NO: 34 - DNA, 22 bp, Mus musculus
cactcagggaatacacgcctac

SEQ ID NO: 35 - DNA, 23 bp, Mus musculus
atcttggtcttagggtcttcagg

SEQ ID NO: 36 - DNA, 20 bp, Mus musculus
cgacactcacccacctcttc

SEQ ID NO: 37 - DNA, 20 bp, Mus musculus
ttgttcagccggatcttgtc

SEQ ID NO: 38 - DNA, 18 bp, Mus musculus
tctgcagatcgcgtggag

SEQ ID NO: 39 - DNA, 19 bp, Mus musculus
cttgtcccggcatagcaac

SEQ ID NO: 40 - DNA, 18 bp, Mus musculus
tcagcaaagcacccgaac

SEQ ID NO: 41 - DNA, 22 bp, Mus musculus
agcgaccatgtctttgtaggag

SEQ ID NO: 42 - DNA, 21 bp, Mus musculus
gctgcggaaacttcaggaaat

SEQ ID NO: 43 - DNA, 23 bp, Mus musculus
agagacgtgtcactcctggactt

SEQ ID NO: 44 - DNA, 20 bp, Mus musculus
aggagtgtcgacttcgcaaa

SEQ ID NO: 45 - DNA, 21 bp, Mus musculus
ctcttcttgccgcttcagttt

SEQ ID NO: 46 - DNA, 20 bp, Mus musculus
atagtgggtcacgaagcagc

SEQ ID NO: 47 - DNA, 20 bp, Mus musculus
agggcaacagagtcggagac

SEQ ID NO: 48 - DNA, 20 bp, Mus musculus
aaggcatggagaggaaggac

SEQ ID NO: 49 - DNA, 21 bp, Mus musculus
agttcgtattggctgggtttc

SEQ ID NO: 50 - DNA, 22 bp, Mus musculus
atcactttggatgtgggaacag

SEQ ID NO: 51 - DNA, 21 bp, Mus musculus
caggaaggcgtcatactcagg

SEQ ID NO: 52 - DNA, 23 bp, Mus musculus
ggagctctcagcttgtgtcttca

SEQ ID NO: 53 - DNA, 25 bp, Mus musculus
caccgatcacaatctctcgtagagt

SEQ ID NO: 54 - DNA, 20 bp, Mus musculus
ggagcttatcccgaacatcc

SEQ ID NO: 55 - DNA, 21 bp, Mus musculus
cggaagacgtccataccattc

SEQ ID NO: 56 - DNA, 20 bp, Mus musculus
ccctgcggatcttccttatc

SEQ ID NO: 57 - DNA, 23 bp, Mus musculus
tgtgtttctgagaacttgtggtg

SEQ ID NO: 58 - DNA, 20 bp, Mus musculus
ggagccatggattgcacatt

SEQ ID NO: 59 - DNA, 18 bp, Mus musculus
ggcccgggaagtcactgt

SEQ ID NO: 60 - DNA, 20 bp, Mus musculus
ccctatcactcctgccacac

SEQ ID NO: 61 - DNA, 22 bp, Mus musculus
acgaagtgcaatggtctttagg

SEQ ID NO: 62 - DNA, 20 bp, Homo sapiens
agtgcttgagcctggcctac

SEQ ID NO: 63 - DNA, 20 bp, Homo sapiens
ttgttcaggcggatcttgtc

SEQ ID NO: 64 - DNA, 18 bp, Homo sapiens
agcggattccaggtgagg

SEQ ID NO: 65 - DNA, 20 bp, Homo sapiens
ttgttcaggcggatcttgtc

(19) United States
(12) Patent Application Publication
(10) Pub. No.: US 2015/0126438 A1
(43) Pub. Date: May 7, 2015

(54) Novel ChREBP Isoforms and Methods Using the Same

(71) Applicant: Beth Israel Deaconess Medical Center, Inc., Boston, MA (US)

(72) Inventors: Mark A. Herman, Needham, MA (US); Barbara B. Kahn, Cambridge, MA (US)

(21) Appl. No.: 14/374,417

(22) PCT Filed: Jan. 22, 2013
(86) PCT No.: PCT/US2013/022521

§ 371 Date: Jul. 24, 2014

Related U.S. Application Data
(60) Provisional application No. 61/590,012, filed on Jan. 24, 2012.

(51) Int. Cl. G01N 33/68; G01N 33/50; C12Q 1/68

(52) U.S. Cl. CPC G01N 33/6893; C12Q 1/6883; G01N 33/502; G01N 2333/47; C12Q 2600/158; C12Q 2600/112; G01N 2800/042; G01N 2800/56; G01N 2800/52; G01N 2500/10; G01N 2500/04
USPC 514/4.8; 536/24.1; 435/6.11; 435/6.12; 506/9; 435/7.92; 436/501; 436/86; 435/7.1; 435/8; 435/29; 514/44 A; 514/19.3; 514/6.9; 514/6.7

(57) ABSTRACT

The invention provides diagnostic and prognostic methods and methods of evaluating treatment protocols for disorders such as obesity, diabetes, metabolic syndrome, cancer and vascular disease by detecting the levels of a novel isoform of ChREBP, termed ChREBP β. The invention also provides nucleic acids, proteins, reporter constructs based on ChREBP β and methods of identifying one or more agents that modulate the expression of a ChREBP β target gene.

### RELATED APPLICATION

This application claims the benefit of U.S. Provisional Application No. 61/590,012, filed on Jan. 24, 2012.

The entire teachings of the above application are incorporated herein by reference.

### GOVERNMENT SUPPORT

This invention was made with government support under R37 DK43051 (B.B.K), K08 DK076726 (M.A.H), and BADERC DK057521 (B.B.K, M.A.H) awarded by the NIH/NIDDK. The government has certain rights in the invention.

### INCORPORATION BY REFERENCE OF MATERIAL IN ASCII TEXT FILE

This application incorporates by reference the Sequence Listing contained in the following ASCII text file being submitted concurrently herewith:

a) File name: SequenceListing.txt; created Nov. 15, 2012, 33 KB in size.

### BACKGROUND OF THE INVENTION

The prevalence of obesity and type 2-diabetes is increasing worldwide and threatens to shorten lifespan. Impaired insulin action in peripheral tissues is a major pathogenic factor. Insulin stimulates glucose uptake in adipose tissue through the Glut4-glucose transporter and alterations in adipose-Glut4 expression or function regulate systemic insulin sensitivity. Downregulation of adipose tissue-Glut4 occurs early in diabetes development. Complications from obesity and type-2 diabetes include vascular disease, which detracts from quality of life further. In addition, cancer, a prevalent and devastating disorder can be characterized by changes in metabolic flux, e.g., via the so-called Warburg effect, by which cancer cells substantially upregulate the level of glycolysis. In view of the prevalence of these disorders and their relation to changes in metabolism, a need exists for methods of detecting and monitoring disease states and/or treatment programs in subjects with diabetes-related disorders, obesity, vascular disease, or cancer, as well as tools and method of identifying agents that modulate key metabolic pathways.

### SUMMARY OF THE INVENTION

The invention provides, inter alia, methods of monitoring disease states and/or treatment response for a variety of metabolic disorders, such as obesity and type 2 diabetes (and diabetes-related disorders) as well as common complications such as vascular disease, and other disorders associated with changes in metabolic flux, such as cancer. The invention also provides isolated nucleic acids and proteins encoding novel isoforms of ChREBP, termed ChREBP β expression products. The invention also provides reporter molecules and methods of screening candidate therapeutic agents for a variety of metabolic disorders as well as cancer and vascular disease. The invention is based, at least in part, on Applicants' surprising discovery of a new mechanism for glucose-regulation of ChREBP: Glucose-mediated activation of the canonical ChREBP isoform (ChREBPα) induces the expression of a novel, potent isoform (ChREBPβ) that is transcribed from an alternative promoter. Expression of ChREBPβ in human adipose tissue predicts insulin sensitivity indicating that it may be an effective target for treating diabetes. Furthermore, in view of the metabolic shift to glycolysis in cancer cells (the Warburg effect) and observations regarding a role for ChREBP α in cell proliferation (see Tong et al., Proc Natl Acad Sci USA. 106(51):21660-5 (2009) (exploring the role of ChREBP α in glucose-dependent anabolic synthesis and cell proliferation)), Applicants' observations on the substantially elevated activity of ChREBP β relative to ChREBP α make levels of ChREBP β expression products important biomarkers for cancer and a target for therapeutic interventions.

Accordingly, in a first aspect, the invention provides methods of assessing the disease state and/or treatment response of a mammalian subject for a disease or disorder selected from, for example, obesity, type 2 diabetes, impaired glucose tolerance, impaired fasting glucose, metabolic syndrome, insulin resistance, vascular disease or cancer. The methods typically include the step of determining the level of a ChREBP β expression product in a biological sample isolated from the mammalian subject, where the level of the ChREBP β expression product is indicative of the subject's disease state and/or treatment response for the disease or disorder. Another aspect of the invention provides methods of treatment for any of the forgoing disorders by providing a suitable prophylaxis or treatment to a subject assessed by the methods provided by the invention, which, in some embodiments, can include either an agonist or antagonist of a ChREBP β expression product.

In another aspect, the invention provides an isolated nucleic acid comprising a ChREBP β expression product or a fragment thereof, which, in some embodiments, may be characterized by comprising a sequence that has at least 60% identity to SEQ ID NO: 1 or a fragment thereof or a similar sequence, e.g., which hybridizes to the complement of SEQ ID NO: 1 under highly stringent hybridization conditions. In another aspect, the invention provides an isolated oligonucleotide capable of specifically amplifying the foregoing nucleic acids. A further aspect of the invention provides a siRNA molecule that specifically targets any of the foregoing nucleic acids.

Another aspect of the invention provides n isolated polypeptide comprising a ChREBP β expression product or a fragment thereof as well as isolated antibodies or antigen-binding fragments thereof that specifically bind such polypeptides.

In yet another aspect, the invention provides screening methods for identifying an agent that modulates the expression of a ChREBP β target gene. The screening methods typically include the step of determining the level of a ChREBP β expression product reporter in a cell contacted with a candidate agent, where a change in the level of the ChREBP β expression product reporter in the cell relative to a control cell not contacted with the candidate agent indicates that the agent modulates the expression of a ChREBP β target gene. In another aspect, the invention also provides isolated nucleic acids comprising a ChREBP β promoter. In certain embodiments, the promoter can provide a useful research tool and includes a nucleic acid sequence that has at least 60% identity to SEQ ID NO: 3 in operative association with a heterologous sequence, e.g., a suitable reporter molecule.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 Genetically altering adipose tissue glucose flux regulates the expression of ChREBP and its lipogenic targets.

FIG. 1a, mRNA expression of fatty acid synthetic enzymes, and FIG. 1b, lipogenic transcription factors in perigonadal fat from 6-week-old female AG4OX, AG4KO, and littermate control mice (n=10-14 per group). *P<0.05 compared to their respective controls.

FIG. 1c, Nuclear abundance of mature Srebp1 and Lamin B1 (loading control) protein in AG4OX and WT adipose tissue from 6-week-old females. Each lane comprises pooled nuclear lysates from 3-4 mice. mRNA of, FIG. 1d, LXR and, FIG. 1e, ChREBP transcriptional targets in perigonadal fat from mice described above. *P<0.05 compared to their respective controls. FIG. 1f, ChREBP and Glut4 mRNA correlate highly in PG WAT from control and AG4KO mice (n=27). R=0.872, P<0.001.

FIG. 2 ChREBP is essential for the effects of adipose tissue Glut4 on adiposity, fatty acid synthesis, and glucose homeostasis. FIG. 2a, Total fatty acid synthesis (from all substrates) measured in vivo in fed, 4-month-old male mice PG=perigonadal, SC=subcutaneous, (n=5-6 per group). FIG. 2b, mRNA expression of fatty acid synthetic enzymes and FIG. 2c, western blots of FAS, ACC, and p85 (loading control) in SC fat from fed, 6-month-old females (n=9-10 per group). For FIGS. 2a-2c, *P<0.05 versus same ChREBP genotype, different AG4OX genotype. #p<0.05 versus same AG4OX genotype, different ChREBP genotype. FIG. 2d, Body weights in male mice on chow (n=5-7 per group). *P<0.05 versus all other groups at the indicated time. FIG. 2e, Body composition in 8-week-old, female mice (for FIGS. 2e-2g; n=10-12 per group). *P<0.01 versus all others. FIG. 2f, Glucose tolerance test and FIG. 2g, insulin tolerance test. *P<0.05 versus all others; #P<0.05 versus WT and ChREBP KO; †P<0.05 versus WT. FIG. 2h, Glycemia following food removal *P<0.05 versus ChREBP KO and AG4OX-ChREBP KO; #P<0.001 versus all others; †P<0.05 versus AG4KO; ‡P<0.05 versus WT and AG4OX-ChREBP KO. FIG. 2i, Glut4 protein levels in PG-fat of 6-month-old females (n=6-8 per group). *P<0.001 within same ChREBP genotype. FIG. 2j, Basal and insulin-stimulated glucose uptake into adipocytes isolated from PG fat (n=6 per group) from male mice. *P<0.05 and ‡P=0.09 by paired t-test for the effect of insulin within genotype. #P<0.05 versus WT and ChREBP KO within same insulin treatment condition.

FIG. 3 ChREBP is regulated in mouse and human adipose tissue in pathologic conditions. FIG. 3a, Body weight and FIG. 3b, composition in chow-versus HFD-WT and AG4OX mice (9-week-old, n=8 per group). For weight,*P<0.05 WT-Chow compared to all others at the same time point. For body composition, *P<0.01 for WT-Chow compared to all others. FIG. 3c, Glucose tolerance test in 7-week-old males (n=8 per group). *P<0.001 versus all others; #P 0.001 versus WT-Chow. FIG. 3d, Total fatty acid synthesis (from all substrates) measured in vivo in 5-6 month-old males (n=7 per group). Last panel, plasma insulin: HFD to increases insulin (†P=0.01 by 2-way ANOVA). FIG. 3e, Glut4 protein levels in PG fat from 4-month-old-males (n=6-8 per group) and mRNA expression of FIG. 3f, ChREBP and SREBP-1c, FIG. 3g, fatty acid synthetic enzymes, FIG. 3h, LXRs and Mlx, and FIG. 3i, LXR transcriptional targets in SC fat from 4-month-old male mice (n=8-14 per group). For FIGS. 3d-3i, *P<0.05 compared to same diet, different genotype. #P<0.05 compared to same genotype, different diet. Correlations between FIG. 3j, ChREBP correlates highly with glucose infusion rate measured during a euglycemic-hyperinsulinemic clamp procedure in non-diabetic, normal glucose-tolerant humans (n=123). FIG. 3k, ChREBP and Glut4 mRNA expression in SC fat correlate in this group (n=123). FIG. 3l, ChREBP mRNA in SC fat correlate highly with % increase in insulin-stimulated glucose uptake over basal measured during a euglycemic-hyperinsulinemic clamp procedure in obese, non-diabetic, BMI-matched humans (n=38). FIG. 3m, ChREBP and Glut4 mRNA expression do not correlate in this group. Due to the large variance and non-normal distribution of Glut4 expression in this group, Log mRNA expression levels were analyzed.

FIG. 4 Expression of the novel ChREBPβ isoform is regulated in a glucose- and ChREBP-dependent manner. FIG. 4a, ChREBP mRNA expression in SC fat from fed, 6-month-old female mice. * and # as described in FIG. 2a legend (n=9-10 per group). FIG. 4b, Model of ChREBPα and ChREBPβ gene structure with indication of splice sites and translational start sites (ATG). FIG. 4c, Regulation of ChREBPα and β mRNA expression in perigonadal fat and liver of 10-week-old, female mice with fasting and refeeding (n=6/group). *P<0.05 compared to fed group. FIG. 4d, ChREBPα and β mRNA expression in perigonadal fat from 6-week-old female AG4OX and AG4KO compared to littermate controls (n=10-14 per group). *P<0.05 versus control. FIG. 4e, Glucose regulation of exon 1b promoter-luciferase reporter and indicated ChREBPβ mutants, co-transfected with ChREBPα and Mlx (n=3/group). *P<0.05 compared to non-mutated exon 1b-luciferase construct in the same glucose. #P<0.05 compared to the same construct in low glucose. FIG. 4f, ChREBPα and β induce an ACC ChoRE-luciferase reporter compared to pGL3_basic control vector in both low and high glucose. *P<0.05 compared to ChREBPα in the same glucose, #P<0.05 compared to ChREBPα, low glucose.

FIG. 5 Glucose-mediated activation of ChREBPα induces expression of ChREBPβ. Glucose or a glucose metabolite stimulates the transcriptional activity of ChREBPα which binds to ChoREs in its lipogenic targets and in ChREBPβ resulting in increased gene expression. The increased ChREBPβ protein further activates expression of ChREBP lipogenic target genes by binding to ChoREs. Whereas glucose regulates ChREBPα transcriptional activity, other nutritional signals regulate its expression. Other nutritional signals may also regulate ChREBPβ expression. The activation of ChREBPα and induction of ChREBPβ expression increase fatty acid synthesis in adipose tissue which improves systemic insulin sensitivity.

FIG. 6 Adipose tissue ChREBPβ expression predicts insulin sensitivity. FIG. 6a, ChREBPα and β mRNA expression in SC fat and liver of 4-month-old male mice on chow or HFD (n=10-14 per group). *P<0.05 compared to Chow-fed. FIG. 6b, mRNA expression of ChREBPβ in SC fat correlates more highly with insulin sensitivity (% increase in insulin-stimulated glucose uptake over basal measured during a euglycemic-hyperinsulinemic clamp procedure) than ChREBPα in obese, non-diabetic, BMI-matched humans (n=38).

FIG. 7. Adipose tissue Glut4, FAS, and ACCT mRNA expression correlate with ChREBP mRNA expression across 30 mouse strains. Each point on the above graphs represents a different mouse strain. Expression data was obtained from the GNF1M Gene Atlas Data set via the BioGPS website (http://biogps.gnf.org)^{22,23}. n=30 mouse strains. Statistics: two-tailed Pearson Correlation.

FIG. 8. Food Intake, energy expenditure, and respiratory exchange ratio measured in AG4OX mice crossbred with ChREBP KO mice. Food intake was measured in individually caged two-month-old female mice over two weeks. Metabolic measurements were made using the Comprehensive Lab Animal Monitoring System (Columbus Instruments). Values are means±SE. n=7-8 per group. Statistical comparisons were performed by ANOVA with pairwise comparisons by Tukey's test. No statistically significant changes were observed.

FIG. 9. Glucose incorporation into newly synthesized fatty acids is normalized in AG4OX adipose tissue lacking ChREBP. Values are means±SE. n=5-6 per group. *p<0.05 compared within genotype, chow to HFD. #p<0.05 compared within diet, WT to AG4OX.

FIG. 10. Liver mRNA expression in 6-month-old female mice. Values are means±SE. n=9-10 per group. *p<0.05 comparing same ChREBP genotype, different AG4OX genotype. #p<0.05 comparing same AG4OX genotype, different ChREBP genotype diet. Using qPCR primers which are proximal to the deleted exons in ChREBP KO mice, ChREBP mRNA can be detected in ChREBP KO tissues.

FIG. 11. Glucose incorporation into newly synthesized fatty acids is reduced in WT and AG4OX mice on HFD in adipose tissue. Values are means±SE. n=7 per group. *p<0.05 compared within genotype, chow to HFD. #p<0.05 compared within diet, WT to AG4OX.

FIG. 12. Liver mRNA expression in 4-month-old male WT mice on chow versus HFD. Values are means±SE. n=10-14 per group. *p<0.05 comparing chow to HFD.

FIG. 13. Position weight matrix for ChoREs. This matrix is generated by counting the frequency of each nucleotide species at each position in the aligned ChoREs of 16 reported and experimentally validated ChoREs in human, mouse, or rat ChREBP targets [Minn, A. H., et al. Endocrinology 146, 2397-405 (2005); O'Callaghan, B. L., et al. J Biol Chem 276, 16033-9 (2001); Pedersen, K. B., et al. Biochem J 426, 159-70; Shih, H. M., et al. J Biol Chem 267, 13222-8 (1992)]

FIG. 14. Conservation of E-box and ChoRE in ChREBP Gene. Sequence is obtained from and elements of the image are adapted from http://genome.ucsc.edu^{47,48}.

FIG. 15. Alignment of genomic sequence obtained from //genome.ucsc.edu^{47,48}. Alignment performed by Clustal W algorithm using Megalign (Dnastar) software. Sequence highlighted in blue corresponds to mouse exon 1b. Sequence highlighted in red corresponds to the upstream E-box. Sequence highlighted in yellow corresponds to the ChoRE. Sequence assemblies and coordinates are as follows: mouse: July 2007 (NCBI37/mm9) Assembly; Chr5(+):135565646-135565996, human: February 2009 (GRCh37/hg19) Assembly; Chr7(−):73062038-73062369, orangutan: July 2007 (WUGSC 2.0.2/ponAbe2) Assembly; Chr7(+):14424980-14425329, dog: May 2005 (Broad/canFam2) Assembly; Chr6(+):9619418-9619774, horse: September 2007 (Broad/equCab2) Assembly; Chr13(−):11282385-11282740, and opossum: October 2006 (Broad/monDom5) Assembly; Chr2(−):530122703-530123068.

FIG. 16. Mono-methylation of lysine 4 of histone H3 (H3kme1) and tri-methylation of lysine 4 of histone H3 (H3kme4) is suggestive of the presence of an active promoter^{49,50}. The peaks in the above graph indicate genomic regions with increased H3K4me1 or H3k4me3. Peaks align under ChREBP exon 1a which is the canonical transcriptional start site for ChREBP. H3k4me1 and H3k4me3 marks are also increased in the genomic region 17 kb 5′ of the canonical ChREBP exon 1a in the Bruce4 embryonic stem cell line and in liver tissue suggesting the presence of an active promoter in this genomic region. ChREBP exon 1b aligns with this region. Image adapted from //genome.ucsc.edu⁴⁷.

FIG. 17. Glucose-dependent expression of the exon 1b-promoter-luciferase construct requires co-transfection of ChREBPα and Mlx in HEK293T cells. (n=3/group). *P<0.05 compared to pGL3 basic in the same glucose condition. #P<0.05 compared to exon 1b-luciferase construct in low glucose. This is representative of three independent experiments.

FIG. 18. Mouse ChREBPα (864 amino acids) and ChREBPβ (687 amino acids) protein structure. NES1 and NES2: nuclear export signals 1 and 2. 14-3-3: binding site for 14-3-3 protein. NLS: nuclear localization sequence. LID: low glucose inhibitory domain. GRACE: glucose response conserved element. Proline-rich region. bHLH/ZIP: basic helix-loop-helix-leucine-zipper. ZIP-like: leucine zipper-like domain [the location of domains within the ChREBP protein adapted from and reviewed in Poupeau, A., et al. Biochimica et Biophysica Acta 1182, 995-1006 (2011)].

FIG. 19. ChREBPα protein levels are markedly higher than ChREBPβ protein levels in cell lysates from luciferase studies (left panel). HEK293T cells were transfected with equivalent moles of ChREBPα versus ChREBPβ expression vectors and co-transfected with Mlx. Equivalent volumes of luciferase lysate were loaded in each lane and equivalent protein loading is verified by ponceau staining (right panel). Blots were probed with rabbit polyclonal anti-ChREBP antibody (Novus Biologicals) then HRPconjugated anti-rabbit secondary antibody (GE Healthcare) and exposed with enhanced chemiluminescence (Perkin Elmer). To visualize the ChREBPβ band a long exposure is required resulting in marked overexposure of the ChREBPα band.

FIG. 20. Western blotting was performed on serial 10-fold dilutions of lysates from ChREBPα transfected cells compared to undiluted lysate from ChREBPβ transfected cells. The lysates are from the cells used for luciferase assays shown in FIG. 4f in the manuscript. Transfection of equivalent moles of ChREBPα and ChREBPβ expression vectors results in 100-fold lower levels of ChREBPβ protein.

FIG. 21. ChREBPβ is localized in the nucleus in low and high glucose conditions. In contrast, ChREBPα is predominantly localized in the cytoplasm in low and high glucose conditions. The panels labeled anti-ChREBP Ab show ChREBPα or ChREBPβ localization in red. The panels labeled Dapi show nuclei in blue. The panels labeled Merged show the merged images of ChREBP and nuclear staining HEK392T cells were transfected with Flag-tagged ChREBPα or ChREBPβ and co-transfected with Mlx overnight in DMEM+10% FBS. Cells were subsequently incubated in DMEM with 2.5 mM glucose and 10% FBS. Cells were either maintained in 2.5 mM glucose or, after 4 hours, changed to 25 mM glucose. Cells were fixed in methanol after 2 hours (shown above) or 18 hours incubation (not shown). ChREBP localization was similar after 2 or 18 hours incubation at both glucose concentrations. Immunofluorescence was performed using a rabbit polyclonal anti-ChREBP antibody (Novus Biologicals) and an Alexa-Fluor 594-conjugated goat anti-rabbit antibody (Invitrogen). Nuclei were stained with Dapi (Vector Systems).

FIG. 22 ChREBPβ is localized in the nucleus in low and high glucose conditions. In contrast, ChREBPα is localized in both the nucleus and extra-nuclear compartment in low and high glucose conditions. HEK392T cells were transfected with Flag-tagged ChREBPα or ChREBPβ and co-transfected with Mlx overnight in DMEM+10% FBS. Cells were subsuquently incubated in DMEM with 2.5 mM glucose and 10% FBS. Cells were either maintained in 2.5 mM glucose or, after 4 hours, changed to 25 mM glucose. Cells were harvested in ice-cold PBS, washed with PBS and then incubated on ice in hypotonic buffer for 10 minutes. NP40 was added to a final concentration of 0.5% and cells were lysed by vortexing. Nuclei were pelleted by centrifugation at 1000 rcf and supernatant (extranuclear lysate) was collected and frozen for further processing. Nuclei were washed in hypotonic buffer, and then resuspended and lysed in RIPA buffer. Protein concentrations for the nuclear and extra-nuclear fractions were determined by BCA assay and equivalent amounts of protein were loaded in each lane (14 ug per lane for nuclear lysates, 30 ug per lane for extra-nuclear lysates). Equivalent loading is verified by ponceau staining (right panel). Blots were probed and developed as described in Fig A. Based upon the calculated amount of total nuclear and extra-nuclear protein, approximately 60% of the nuclear protein recovered from each sample was loaded per lane. Approximately 10% of the extra-nuclear protein was loaded per lane. The ChREBPα band is roughly 3-fold more intense in the nuclear compared to the extra-nuclear lysate. Thus, based upon these blots, there is ˜2-fold more ChREBPα in the extranuclear compartment compared to the nuclear compartment irrespective of the glucose condition. Even with longer exposure, ChREBPβ is not seen in the extra-nuclear fraction.

### DETAILED DESCRIPTION OF THE INVENTION

A description of example embodiments of the invention follows.

### I. Certain Definitions

The following definitions will be adhered to throughout the application.

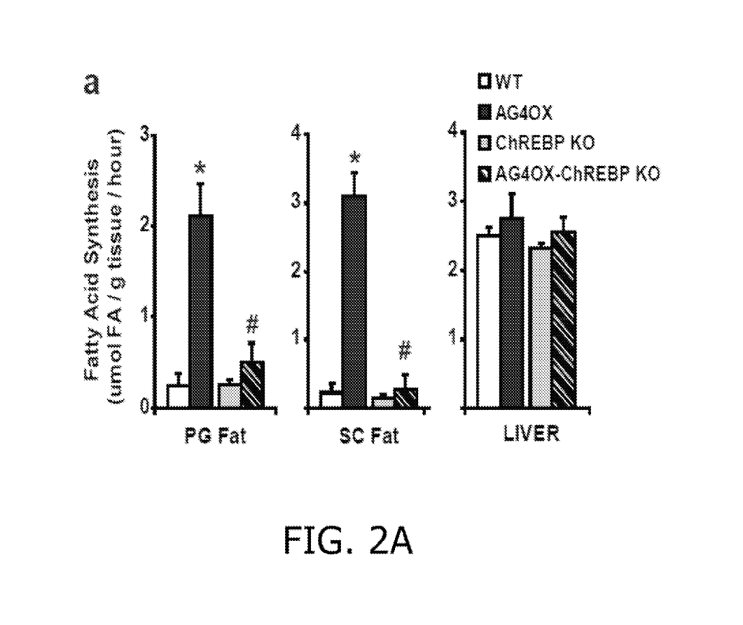

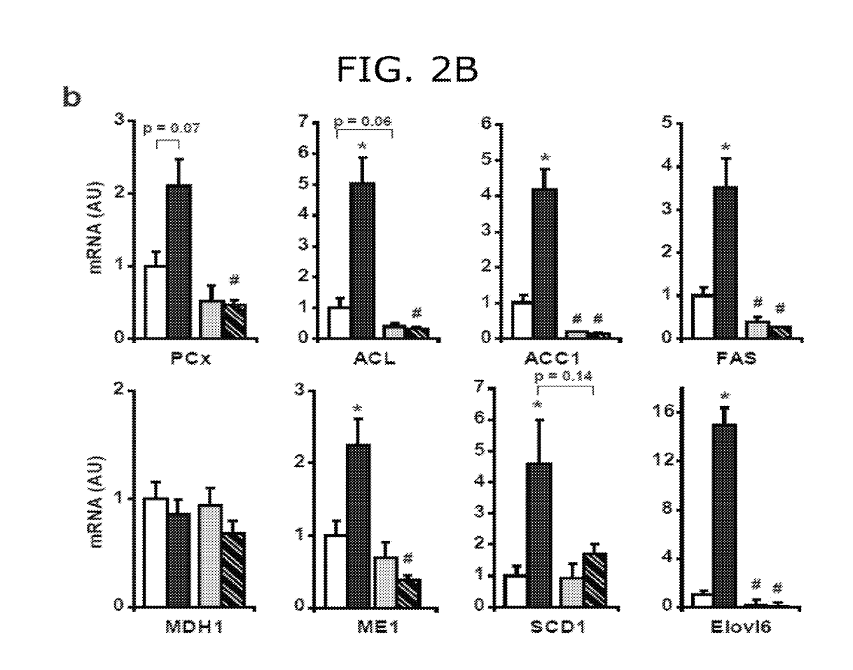

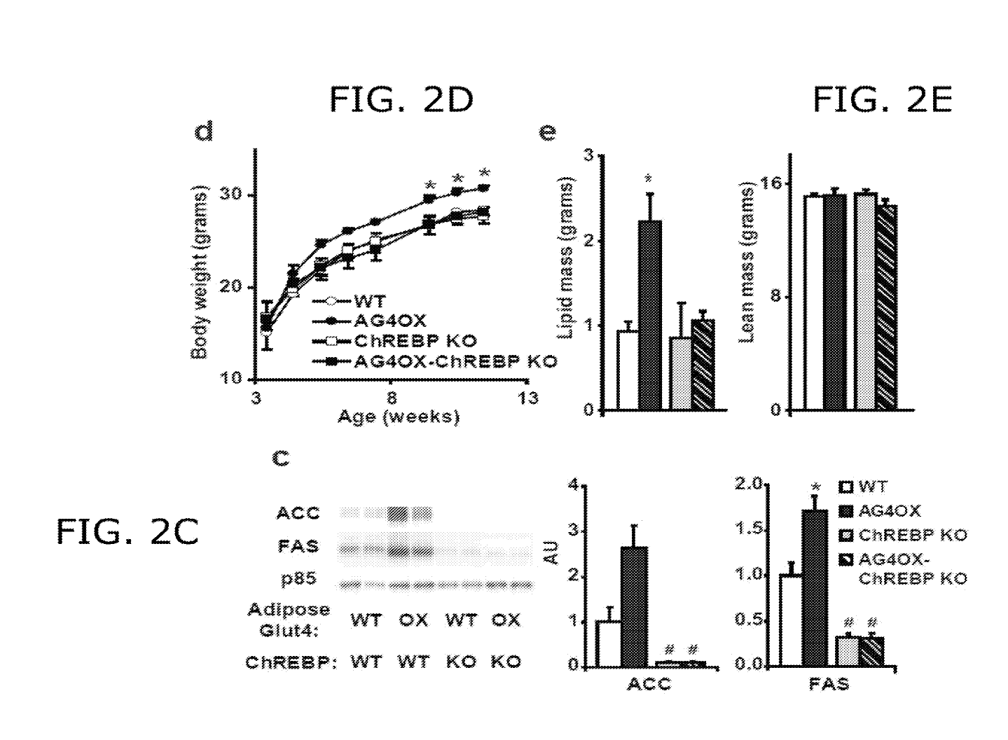

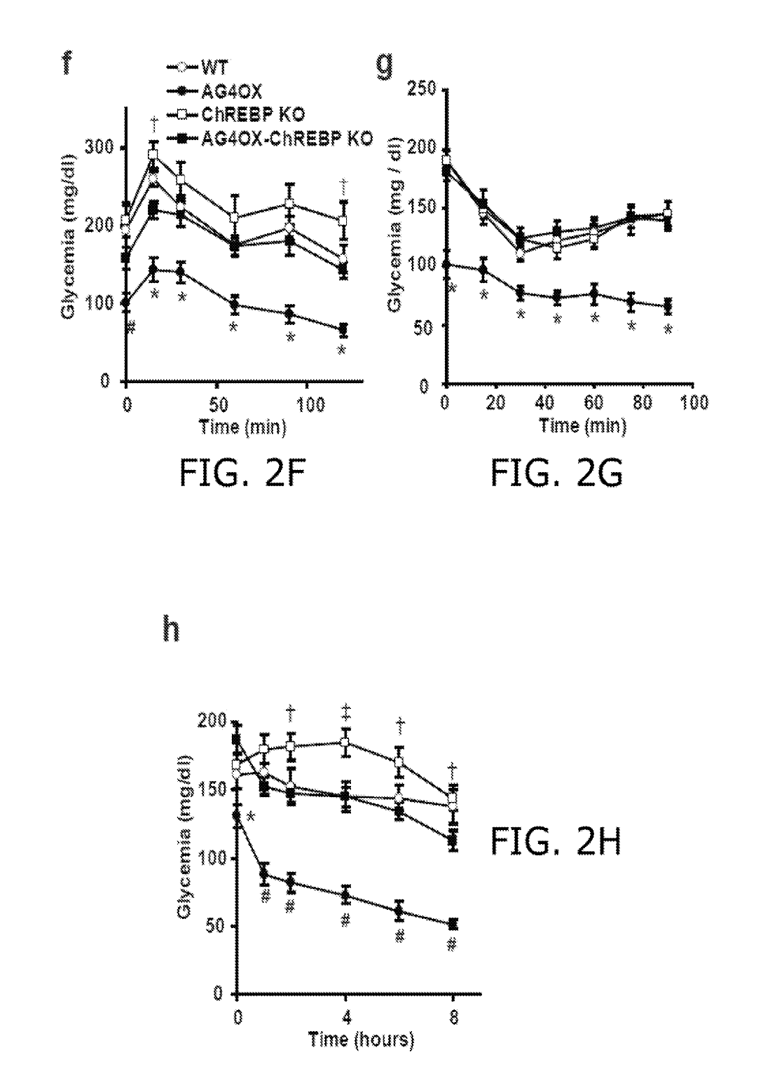

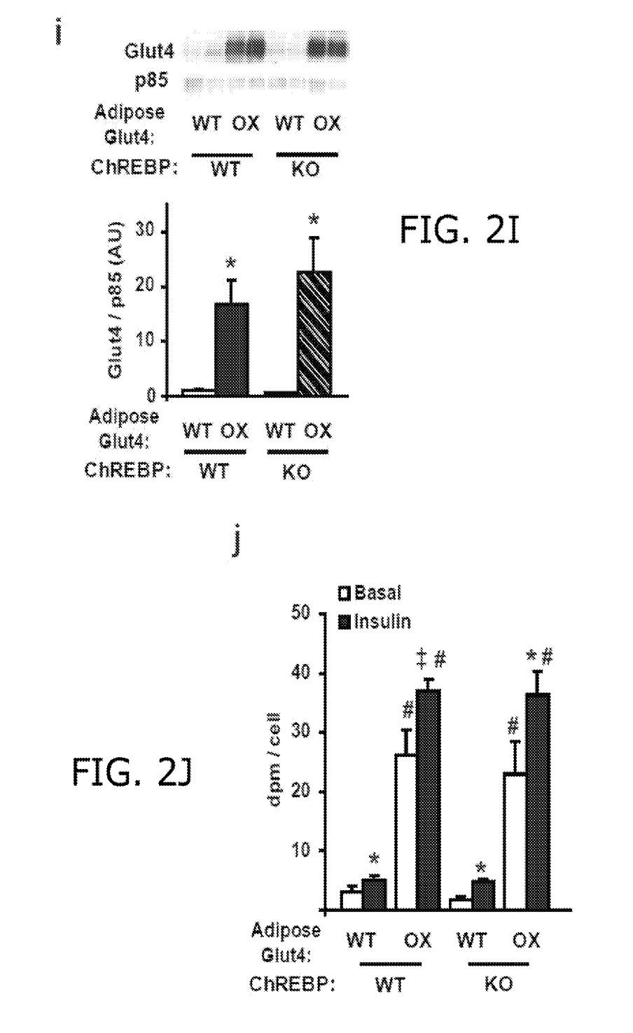

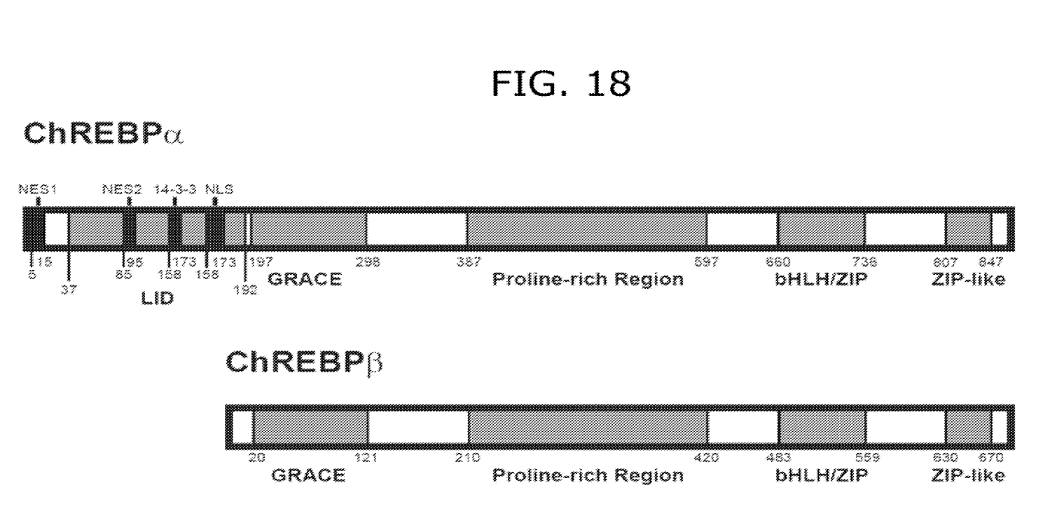

“ChREBP β expression product” or simply “ChREBP β” refers to a non-recombinant nucleic acid encoding an N-terminally truncated isoform of ChREBP that exhibits enhanced transcriptional activation relative to non-recombinant ChREBP protein that is not N-terminally truncated—termed “ChREBP α,” here; “ChREBP β” and “ChREBP β expression product” also encompasses a protein expression product encoded by non-recombinant nucleic acid which encodes an N-terminally truncated isoform of ChREBP that exhibits enhanced transcriptional activation relative to ChREBP α. In a particular embodiment, ChREBP α is exemplified by the reference sequence NP_{—}116569. In some embodiments a ChREBP β expression product has (or, in the case of a nucleic acid, encodes) an N-terminal truncation, relative to ChREBP α, of at least about 10, 20, 30, 40, 50, 75, 100, 125, 150, 170, 171, 172, 173, 174, 175, 176, 177, 178, 179, or 180 amino acids. In certain embodiments, the ChREBP β expression product is encoded by a transcript lacking the first exon of a ChREBP transcript encoding a ChREBP α protein (such as the reference sequence NM_{—}032951.2). In more particular embodiments the ChREBP β expression product is encoded by a transcript comprising an alternative first exon, termed “exon 1b” herein, preferably wherein the exon 1b does not encode protein sequence. In more particular embodiments, the exon 1b comprises a nucleic acid sequence having at least 60% identity to SEQ ID NO: 1 or 5. ChREBP β expression products will typically retain the function of structurally conserved regions, including those depicted in FIG. 18 and, in more preferred embodiments, which preserve the function of the “P2” domain depicted in Li et al. 2006 (see FIG. 2 in Li, which is incorporated herein by reference, and is contained within the GRACE domain), which is required for ChREBP function.

“Abnormal levels of a ChREBP β expression product” may be either aberrantly high or aberrantly low, depending on the particular subject, disorder, or tissue, and may be assessed by a variety of means including concentration of the expression product that is normalized to volume and/or, for example, amount or concentration of total protein, a reference nucleic acid, protein or other metabolite, as well as a ratio of concentration to ChREBP α expression products. Abnormal levels are determined relative to suitable controls, which can be assessed by any means including, for example, paired samples from a single patient (e.g., samples obtained at different times, e.g., before and after developing a disorder; as well as a pair of samples from different tissues, which may be obtained at the same or different times) as well as reference values previously compiled from samples determined—by any means—to be associated with, e.g., a diseased or normal state. For example, reference values for one or more disorders and/or treatments may be compiled and used to develop binary or probabilistic classification algorithms that are then used to classify a sample from a patient as, e.g., normal or abnormal.

“Gene expression” or “expression product” encompasses both nucleic acid (e.g., mRNA or cDNA derived from it) and protein expression of a gene. Nucleic acid expression products may or may not include subsequences that do not encode and/or get translated into protein.

“Level of expression,” “expression level,” “gene expression level” and the like, refers to the amount of a gene expression product (e.g., mRNA or protein) and may be normalized by any suitable means.

“ChREBP β expression product reporter” is an expression product—at the nucleic acid or protein level—whose level is modulated following induction of a ChREBP β expression product in a cell or non-cellular system and encompasses both recombinant and non-recombinant products.

“ChREBP β target gene” is a non-recombinant ChREBP β expression product reporter.

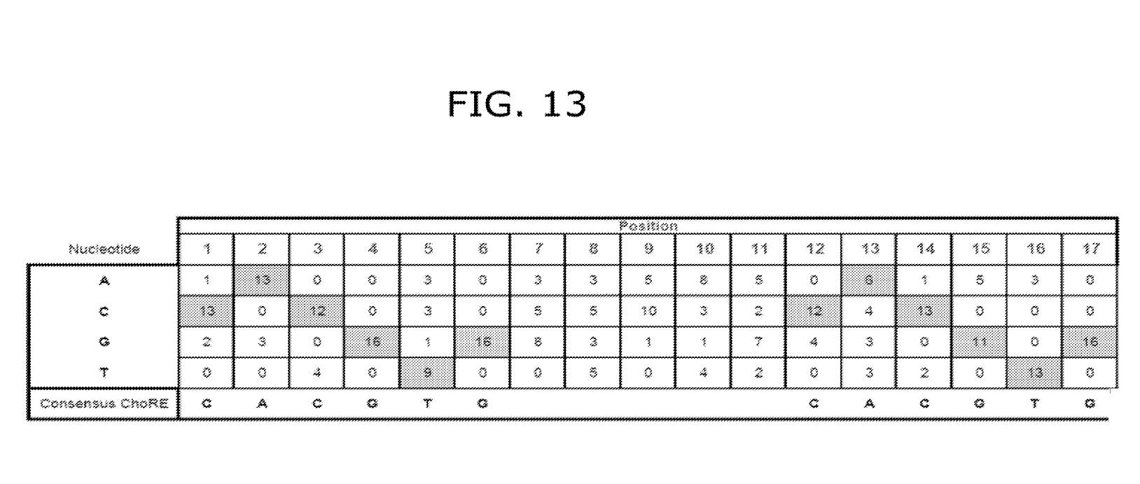

A “ChREBP β promoter” is a nucleic acid comprising an exon 1b of a ChREBP β expression product and further comprising at least one of an E-box element or a ChoRE, both of which are exemplified in FIGS. 13, 14 and 15. In particular embodiments a ChREBP β promoter contains both an E-box element and a ChoRE. In more particular embodiments, the exon 1b has at least 60% identity to SEQ ID NO: 1 or 5. In still more particular embodiments, a ChREBP β promoter comprises a sequence having at least 60% identity to SEQ ID NO: 3 or 7.

A “subject” refers to a mammal, including primates (e.g., humans or monkeys), cows, sheep, goats, horses, dogs, cats, rabbits, guinea pigs, rats, mice or other bovine, ovine, equine, canine, feline, rodent or murine species. Examples of suitable subjects include, but are not limited to, human patients (e.g., obese, diabetic, non-diabetic, having a diabetes-related disorder, cancer or vascular disease) and in more particular embodiments human patients (e.g., obese, non-obese) who have, or are at risk for developing, insulin resistance, type 2 diabetes, metabolic syndrome, vascular disease or cancer. Examples of high-risk groups for the development of metabolic syndrome, insulin resistance or type 2 diabetes include medically overweight and obese individuals. In preferred embodiments, the subject is human. In particular embodiments, the subjects to be tested or treated by the methods provided by the invention have, or are at increased risk for developing obesity or a diabetes-related disorder, cancer or vascular disease. In more particular embodiments, the vascular disease may be secondary to either obesity and/or a diabetes-related disorder. Similarly, the diabetes-related disorder may be secondary to obesity, or vice-versa. While subjects may be of any stage of life and any age, e.g., neonate, infant, toddler, child, young adult, adult, or geriatric; in particular embodiments the subject is an adult, e.g. a human adult, i.e. 18 years old, or older, e.g., 18-70, 20-60, 25-55, 25-50, 30-50, 25-65 years old, as well as greater than 30, 40, 50, 60, 70, 80 or 90 years old.

As used herein, the terms “treat,” “treating,” or “treatment,” mean to counteract a medical condition (e.g., obesity, a diabetes-related disorder, cancer, vascular disease) to the extent that the medical condition is improved according to a clinically-acceptable standard. For example, an improvement in a medical condition related to obesity can be determined according to one or more of the following: 1) reduction of body weight, 2) reduction of body mass index (BMI), 3) reduction of waist-to-hip ratio (WHR); improvement relative to diabetes can include 1) improved glucose tolerance, 2) reduced glycated hemoglobin, 3) improved insulin sensitivity, 4) improved glycemia; improvement in cancer can include: 1) reduced tumor growth, 2) tumor shrinking, 3) remission, 4) reduction in metastases, 5) reduced glucose uptake or utilization; improvement in vascular disease can include 1) reduced blood pressure, 2) lowered LDL cholesterol, 3) increased HDL cholesterol, 4) lowered triglycerides, 5) reduced atherosclerotic burden, 6) improved cardiac output.

The terms “prevent,” “preventing,” or “prevention,” as used herein, mean reducing the probability/likelihood, progression, onset, risk or severity of a disorder—including, for example, obesity or a diabetes-related disorder, cancer or vascular disease—in a subject. In general, a subject undergoing a preventative regimen most likely will be categorized as being “at-risk” for a given disorder, e.g., the risk for the subject developing obesity, a diabetes-related condition, vascular disease or cancer is higher than the risk for an individual represented by the relevant baseline population.

As used herein, a “therapeutically effective amount” is an amount sufficient to achieve the desired therapeutic or prophylactic effect under the conditions of administration, such as an amount sufficient to inhibit (e.g., reduce, prevent), e.g., obesity, diabetes-related disorder, vascular disease or cancer. The effectiveness of a therapy can be determined by one skilled in the art using standard measures and routine methods.

The term “obese” or “obesity” refers to the condition of a subject having a body mass index (BMI) of about 30 kg/m² or higher, e.g., a BMI of 25, 26, 27, 28, 29, 30, 31, 32, 33, 34, 35, 36, 37 kg/m², or more. In particular embodiments, an obese subject has a BMI within the ranges defined as “obese” by the Center for Disease Control. See, URL //www.cdc.gov/obesity/defining.html. For example, in some embodiments, an adult who has a BMI >=30.0 kg/m² is obese.

“Diabetes-related disorders” include T2D, impaired fasting glucose, impaired glucose tolerance, insulin resistance and metabolic syndrome.

“Type 2 diabetes” or “T2D” (OMIM 125853), in some embodiments, is defined as provided by the World Health Organization and the International Diabetes Federation in “Definition and diagnosis of diabetes mellitus and intermediate hyperglycaemia,” published in 2006, which is incorporated by reference in its entirety. In more particular embodiments, a diabetic subject exhibits a fasting plasma glucose of >=126 mg/dL or a 2-hour plasma glucose (2 hours after oral administration of 75 grams of glucose) >=200 mg/dL. In some embodiments a diabetic or pre-diabetic subject exhibits elevated levels of glycated hemoglobin, e.g., greater than 4.5, 4.6, 4.7, 4.8, 4.9, 5.0, 5.1, 5.2, 5.3, 5.4, 5.5, 5.6, 5.8, 6.0, 6.2, 6.4, 6.6, 6.8, 7.0, 7.2, 7.4, 7.6%, or more of total hemoglobin.

“Insulin resistance,” which may be identified by any means known in the art, and is characterized by a reduced ability of insulin to lower blood glucose levels.

The term “metabolic syndrome” refers to a group of symptoms that occur together and increase the risk for coronary artery disease, stroke and type 2 diabetes. In some embodiments the subject has central obesity (waist circumference >=80 cm for women; >=90 cm for Asian men, including ethnic South and Central Americans, and >=94 cm for all other males), BMI>30 kg/m², raised triglycerides (>=150 mg/dL, or specific treatment for this lipid abnormality), reduced HDL cholesterol (<40 mg/dL in males, <50 mg/dL in females or specific treatment for this lipid abnormality), raised blood pressure (sBP>=130 mm HG or dBP>=85 mm HG or treatment of previously diagnosed hypertension) or raised fasting plasma glucose (FPG>=100 mg/dL or previous type 2 diabetes diagnosis), including combinations thereof. In more particular embodiments, the subject to be treated by the methods provided by the invention has or is at increased risk for metabolic syndrome, as defined by the International Diabetes Federation in “The IDF consensus worldwide definition of the metabolic syndrome,” published in 2006, which is incorporated by reference in its entirety, i.e., the subject has central obesity (as described above, and/or BMI>30 kg/m²) AND any two of raised triglycerides, reduced HDL cholesterol, raised blood pressure, or raised fasting plasma glucose.

“Cancer” refers to mammalian cancers, in some embodiments, human cancers, and carcinomas, sarcomas, adenocarcinomas, lymphomas, leukemias, et cetera, including solid and lymphoid cancers, kidney, breast, lung, kidney, bladder, colon, ovarian, prostate, pancreas, stomach, brain, head and neck, skin, uterine, testicular, esophagus, and liver cancer, including hepatocarcinoma, lymphoma, including non-Hodgkin's lymphomas (e.g., Burkitt's, Small Cell, and Large Cell lymphomas) and Hodgkin's lymphoma, leukemia, and multiple myeloma. Cancers embraced in the current application include both metastatic and non-metastatic cancers. In certain embodiments, a cancer cell may exhibit one or more of loss of contact inhibition when cultured, abnormal karyotype, abnormal cellular morphology or altered metabolic state consonant with what is termed the Warburg effect. Additional states that may be related to cancer and that can be diagnosed, monitored and/or treated by the methods provided by the invention include precancerous lesions and neoplasias.

“Vascular disease” is a pathological state of large, medium, or small sized arteries and may be triggered by endothelial cell dysfunction (e.g. including aneurisms, blockage, collapse) in central, peripheral or cerebral vasculature and can include angina, as well as severe complications such as stroke (ischemia), myocardial infarct (heart attack), arrhythmia, congestive heart failure, or ischemia resulting in gangrene or amputation.

“Highly stringent hybridization” conditions refers to at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

The term “antibody,” as used herein, refers to an immunoglobulin or a part thereof, and encompasses any polypeptide comprising an antigen-binding site regardless of the source, species of origin, method of production, and characteristics. Antibodies for use in the methods provided by the invention include, for example, human, orangutan, mouse, rat, goat, sheep, rabbit and chicken antibodies. Antibodies may be polyclonal, monoclonal, monospecific, polyspecific, non-specific, humanized, camelized, single-chain, chimeric, synthetic, recombinant, hybrid, mutated, and CDR-grafted antibodies. For the purposes of the present invention, antibodies also include, unless otherwise stated, antibody fragments such as Fab, F(ab′)2, Fv, scFv, Fd, dAb, VHH (also referred to as nanobodies), and other antibody fragments that retain the antigen-binding function. Antibodies will have at least 3 CDRs and, in more particular embodiments, 4, 5, or 6 CDRS.

### II A. ChREBP α and ChREBP β

The present invention relates to novel forms of carbohydrate responsive-element binding protein, ChREBP, also known as MlxIPL (Mlx interacting protein-like, see OMIM 605678), MIO, MONDOB, WBSCR14, WS-bHLH and bHLHd14. ChREBP sequences from a variety of organisms are known, as shown in TABLE A.

TABLE A

| Organism | Entrez GeneID |
|---|---|
| Human | 51085 |
| Mus musculus | 58805 |
| Bos taurus | 788534 |
| Pongo abelii | 100438130 |
| Pan troglodytes | 742401 |
| Gallus gallus | 425603 |
| Canis lupus familiaris | 489807 |
| Rattus norvegicus | 171078 |
| Sus scrofa | 100170769 |
| Cricetulus griseus | 100760281 |

The GeneIDs in TABLE A can be used to retrieve reference protein and mRNA sequences from the NCBI (National Center for Biotechnology Information) database, as well as genomic sequences, along with annotations of each of these sequences for ChREBP α expression products—i.e. expression products that may include, e.g., differentially spliced variants—but none of which include the unprecedented ChREBP β expression products discovered by Applicants. For example, Table B shows reference protein and mRNA sequences available under some of the GeneIDs in Table A.

TABLE B

| Organism | mRNAs | Proteins |
|---|---|---|
| Human | NM_032951.2 | NP_116569.1 |
| | NM_032952.2 | NP_116570.1 |
| | NM_032953.2 | NP_116571.1 |
| | NM_032954.2 | NP_116572.1 |
| mouse | NM_021455.4 | NP_067430.2 |
| rat | NM_133552.1 | NP_598236.1 |
| pig | XM_003481002.1 | XP_003481050.1 |
| | XM_003124408.2 | XP_003124456.2 |
| chimp | XM_003318668.1 | XP_003318716.1 |
| | XM_003318669.1 | XP_003318717.1 |

The present invention is based, at least in part, on Applicants' unexpected and surprising discovery of novel forms of ChREBP, termed ChREBP β expression products, as defined above. The working examples of the application summarize observations made in mouse and human cells and provide exemplary mRNAs, proteins and promoters for human and mouse ChREBP β, which are provided in SEQ ID NOs: 1-8 as summarized in TABLE C, below.

TABLE C

| SEQ ID NO: | descriptor |
|---|---|
| 1 | human exon 1b of ChREBP β |
| 2 | human ChREBP β protein |
| 3 | human ChREBP β promoter |
| 4 | human ChREBP β mRNA |
| 5 | murine exon 1b of ChREBP β |
| 6 | murine ChREBP β protein |
| 7 | murine ChREBP β promoter |
| 8 | murine ChREBP β mRNA |

Based on Applicants' observations, variants of the exemplary ChREBP β expression products listed in TABLE C, including ChREBP β expression products from other species, can be readily detected.

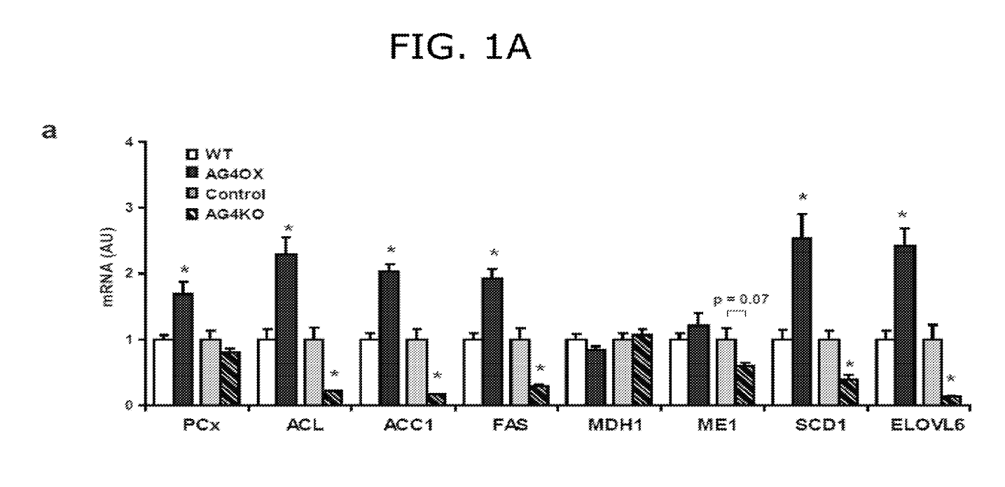

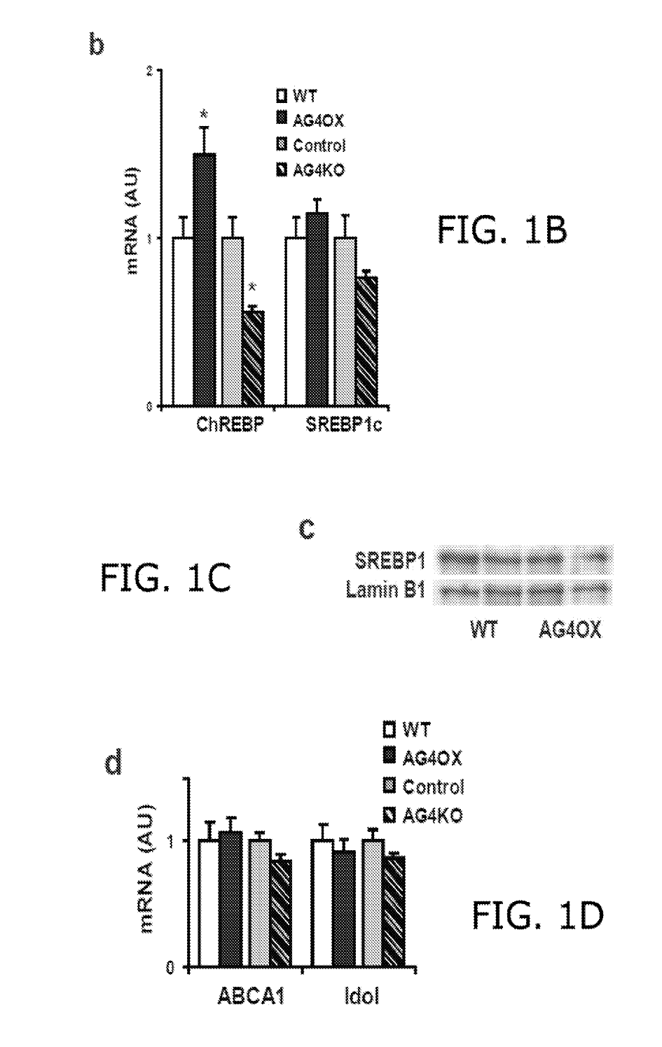

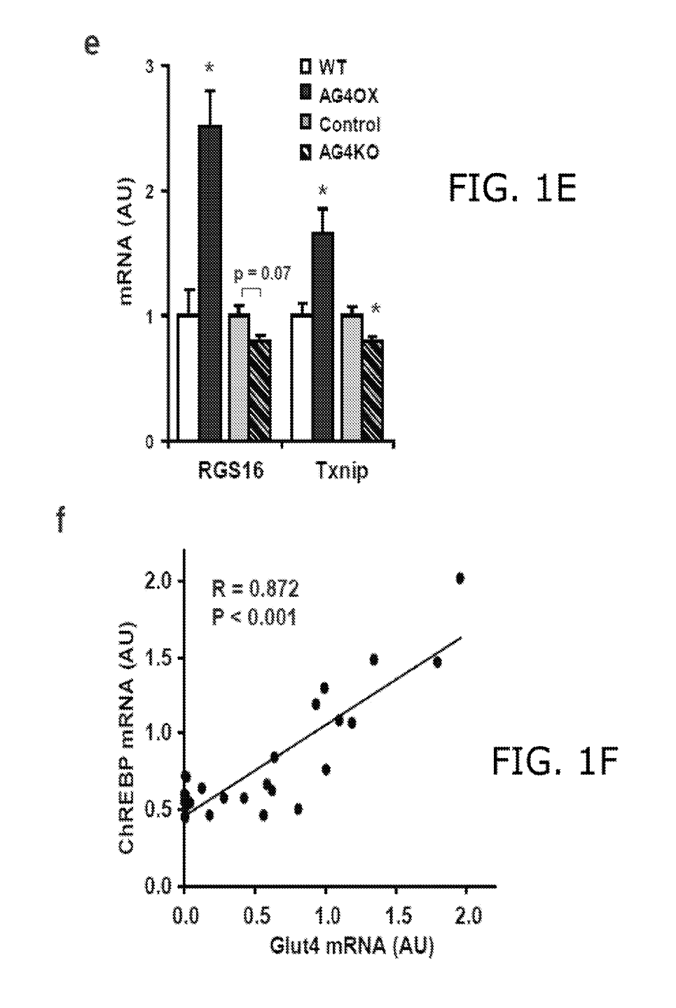

For example, before the present invention, the ENTREZ database annotated 4 splice variants of human ChREBP α, which result in slightly different protein sequences in the C-terminus of the proteins, but none of which encompass the ChREBP β expression products provided by the invention. Therefore, in certain embodiments, ChREBP β expression products encompasses N-terminal truncations of each of above human splice variants, concordant with Applicants' observations in mouse. In a similar fashion, ChREBP β expression products from other species can be easily identified by performing sequence alignments (pairwise or multiple sequence alignments—by any means), and making reference to, for example, the reference nucleic acid and protein structures illustrated in FIGS. 13-15 (nucleic acids) and FIG. 18 (protein, including conserved domain structures). Additional information on the structure of ChREBP proteins is provided in Poupeau and Postic Biochim Biophys Acta. 1812(8):995-1006 (2011; particularly FIG. 1 therein) and Li et al., Diabetes 55:1179-89 (2006), which are incorporated by reference in their entirety.

Methods for sequence alignments and comparison are widely known and include FASTA (Lipman and Pearson, Science, 227: 1435-41 (1985) and Lipman and Pearson, PNAS, 85: 2444-48), BLAST (McGinnis & Madden, Nucleic Acids Res., 32:W20-W25 (2004) (current BLAST reference, describing, inter alia, MegaBlast); Zhang et al., J. Comput. Biol., 7(1-2):203-14 (2000) (describing the “greedy algorithm” implemented in MegaBlast); Altschul et al., J. Mol. Biol., 215:403-410 (1990) (original BLAST publication)), Needleman-Wunsch (Needleman and Wunsch, J. Molec. Bio., 48 (3): 443-53(1970)), Sellers (Sellers, Bull. Math. Biol., 46:501-14 (1984), and Smith-Waterman (Smith and Waterman, J. Molec. Bio., 147: 195-197 (1981)), and other algorithms (including those described in Gerhard et al., Genome Res., 14(10b):2121-27 (2004)), which are incorporated by reference.

For simplicity, ChREBP β expression products claimed or used in methods provided by the invention may be described by reference (e.g. by percent identity or homology) to any of the suitable reference sequences in TABLE C as well as ChREBP β sequences readily deduced from any of the identifiers provided in TABLES A and B, above. For example, in certain embodiments, a protein ChREBP β expression product comprises an amino acid sequence having at least 60% identity to SEQ ID NO: 2 or 6, e.g., at least 60, 65, 70, 75, 80, 85, 90, 95, 96, 97, 98, 99, or 100% identity to SEQ ID NO: 2 or 6. In particular embodiments, the protein ChREBP β expression product can be characterized as lacking a functional “LID” domain, as shown in FIG. 18, and described further in Li et al., 2006. Similarly, in some embodiments, a nucleic acid ChREBP β expression product comprises a sequence having at least 60% identity to any one of SEQ ID NOs: 1, 4, 5 or 8, e.g., at least 60, 65, 70, 75, 80, 85, 90, 95, 96, 97, 98, 99, or 100% identity to any one of SEQ ID NOs: 1, 4, 5 or 8—e.g. exon 1b's may be described relative to SEQ ID NOs: 1 and 5 and mRNAs (or corresponding cDNAs) references may be described relative to SEQ ID NOs: 4 and 8 (mRNAs). ChREBP β promoters may be described by reference to SEQ ID NOs: 3 and 7, analogously, e.g. having at least 60, 65, 70, 75, 80, 85, 90, 95, 96, 97, 98, 99, or 100% identity thereto. Of course, the invention encompasses ChREBP β expression products, exon 1bs, and ChREBP β promoters defined relative to other reference sequences, including those from any organism listed in TABLE A as well as those based on the ChREBP α expression products exemplified in TABLE B—e.g. by performing sequence alignments to the ChREBP β expression products, exon 1bs, and ChREBP β promoters exemplified here, such as those in TABLE C.

Accordingly, in one aspect, the invention provides isolated nucleic acid comprising a ChREBP β expression products or a fragment thereof, wherein the fragment is of a size suitable to distinguish it from other nucleic acids in a biological sample. In particular embodiments, the fragment is at least about 10, 15, 16, 17, 18, 19, 20, 21, 22, 23, 24, 25, 26, 27, 28, 29, 30, 35, 40, 45, 50, 55, 60, 70, 80, 90, 100, 110, 120, 130, 140, 150, 160, 170, 180, 190, 200, 210, 220, 300, 400, 500, 600, 700, 800, 900, 1000, 1250, 1500, 1750, 2000, 2250, 2500, 2750, 3000, 3050, 3100 nucleotides, or more, in length and, in particular embodiments, comprises a nucleic acid that has at least 60% identity to SEQ ID NO: 1 or 5 or a fragment thereof, e.g. at least 60, 65, 70, 80, 85, 90, 95, 96, 97, 98, 99 or 100% identity to a fragment of at least about 10, 15, 16, 17, 18, 19, 20, 21, 22, 23, 24, 25, 26, 27, 28, 29, 30, 35, 40, 45, 50, 55, 60, 70, 80, 90, 100, 110, 120, 130, 140, 150, 160, 170, 180, 190, 200 nucleotides. In other embodiments, the nucleic acid comprises a sequence hybridizes under highly stringent conditions to SEQ ID NO: 1 or 5 or its complement.

In another aspect, the invention provides siRNAs that specifically target nucleic acid ChREBP β expression products and in particular embodiments, specifically target a nucleic acid ChREBP β expression product comprising a nucleic acid sequence that has at least 60% identity to SEQ ID NO: 1 or 5 or a fragment thereof, e.g. at least 60, 65, 70, 80, 85, 90, 95, 96, 97, 98, 99 or 100% identity to a fragment of at least about 10, 15, 16, 17, 18, 19, 20, 21, 22, 23, 24, 25, 26, 27, 28, 29, 30, 35, 40, 45, 50, 55, 60, 70, 80, 90, 100, 110, 120, 130, 140, 150, 160, 170, 180, 190, 200 nucleotides of SEQ ID NOs: 1 or 5. Methods of designing siRNAs are well known in the art and described, inter alia, in U.S. Pat. Nos. 7,078,196; 7,056,704, U.S. Patent Application Publication No. 20090209625 and International Publication Numbers WO 2001/075164 and WO 2002/044321, which are incorporated by reference.

In another aspect, the invention provides isolated polypeptides comprising a protein ChREBP β expression product or a fragment thereof. In particular embodiments, the fragment is at least about 10, 20, 30, 40, 50, 60, 70, 80, 90, 100, 150, 200, 250, 300, 350, 400, 450, 500, 550, 600, or 650 amino acids in length. In particular embodiments, the polypeptide comprises an amino acid sequence has at least 60% identity to SEQ ID NO: 2 or 6, e.g., at least least 60, 65, 70, 80, 85, 90, 95, 96, 97, 98, 99 or 100% identity. The invention also provides dominant negative ChREBP proteins based on protein ChREBP β expression products—e.g., those lacking a functional DNA binding domain, e.g., due to deletion or mutation. The DNA binding domain of ChREBP is contained in the bHLH domain depicted in FIG. 18, which corresponds to approximately amino acids 483-559 of SEQ ID NO: 6.

### II B. Certain Sequences

SEQ ID NO: 1 is a human ChREBP β exon 1b nucleic acid sequence.

SEQ ID NO: 2 is a human ChREBP β protein sequence.

SEQ ID NO: 3 is the genomic sequence 500 base pairs 5′ and 3′ to human ChREBP β exon 1b. The upstream e-box is nucleotides 418 to 423. The ChoRE is nucleotides 653 to 669. The exon 1b sequence is nucleotides 501 to 695. In the human genomic exon 1b region there is an additional e-box, nucleotides 693 to 698, and a potential ChoRE, nucleotides 1075 to 1091, that is conserved across primates, but not present in the mouse genomic sequence.

SEQ ID NO: 4 is a full-length human ChREBP β mRNA. The sequence of Exon 1b is nucleotides 1 to 195.

SEQ ID NO: 5 is a murine exon 1b nucleic acid sequence.

SEQ ID NO: 6 is a protein sequence of murine ChREBP β.

SEQ ID NO: 7 is a murine ChREBP β promoter nucleic acid sequence. This is the genomic sequence 500 base pairs 5′ and 3′ to murine exon 1b. The upstream e-box is nucleotides 403 to 408. The ChoRE is nucleotides 658 to 674. The exon 1b sequence is nucleotides 501 to 671.

SEQ ID NO: 8 is a full-length murine ChREBP β mRNA nucleic acid sequence. The Exon 1b sequence is nucleotides 1 to 171.

### III. Diagnostic Methods, Oligonucleotides, Antibodies and Kits

The invention provides a variety of diagnostic, prognostic and monitoring (e.g. disease progression and/or treatment efficacy) by determining the level of ChREBP β expression products in a biological sample from a subject and, e.g., comparing them to suitable controls, such as annotated reference values for a particular disease or disorder, as well as monitoring them over time in a subject. Accordingly, the invention also provides kits, nucleic acids (e.g. oligonucleotides, including pairs of oligonucleotides for amplification) and antibodies for performing these methods.

Based on the prominent role of ChREBP in metabolism—including glucose flux and lipid metabolism—and the central role of these metabolic pathways in obesity, diabetes-related disorders, cancer and vascular disease, and further in view of the dramatic activity of ChREBP β expression products, the level of ChREBP β expression products is a powerful monitor of disease state and, accordingly, can elucidate the need, efficacy and/or progress of any treatments for these disorders.

For the methods provided by the invention, fragments of ChREBP β expression products (nucleic acid or protein) detected need not be functional per se, as these molecules are being used in such applications as biomarkers only. Thus, fragments of ChREBP β expression products detected by the methods provided by the invention described under this subheading, in general, need only be of a size and structure so as to be distinguishable from other molecules in a given biological sample (or derived fraction thereof), such as total protein or nucleic acid, or ChREBP α expression products.

As defined above, any mammalian subject can be evaluated by the methods of the invention, while human subjects are one particular exemplification. Also, the subject may be of any age, with adult human subjects serving as particular exemplifications. However veterinary applications, particularly in a research context to develop treatments for human subjects are clearly encompassed by the invention as well.

Samples for use in the methods provided by the invention include any suitable biological sample or fraction thereof (e.g., extracted total nucleic acids, mRNAs, cDNAs, total protein, or mixture or further subfractions thereof). In particular embodiments, the biological sample may be isolated from blood, liver, adipose tissue, brown adipose tissue, muscle, pancreas, islet cells, kidney, breast, small intestine, bone marrow, nervous tissue (central, including brain or spine, and/or periperheral), ovary, or prostate. In some embodiments, the biological sample includes cancerous, precancerous, or neoplastic tissue that may include tissue obtained from any of the foregoing tissues. In more particular embodiments, the biological sample is obtained from liver or adipose tissue. Biological samples may be assayed from fresh or fixed samples.

The classification of a sample as normal (or well-controlled) or associated with a disease state (and/or requiring a modified treatment protocol) depends on the particular indication being assayed for (e.g., cancer versus obesity and diabetes-related disorders, including, in some embodiments, associated vascular disease). For example, elevated levels of ChREBP β expression products in liver or in cancerous, precancerous, or neoplastic samples are associated with disease states and/or indicate a need for modified treatment regimens. With regard to cancer, this may be based, at least in part on what is termed the Warburg effect—where cancer cells shift their metabolic profiles towards glycolysis and lactic acid fermentation. See, e.g., Christofk et al., Nature 452(7184):230-33 (2008); see also Tong et al. (2009). Furthermore, the activity of ChREBP to promote lipogenesis may be important for cancer progression. Tong et al., (2009). Expression of lipogenic enzymes positively correlated with ChREBP activity such as ATP-citrate lyase, acetyl-CoA carboxylase, and fatty acid synthase are increased in many cancers and inhibiting the activity of these enzymes may be therapeutic or preventative for numerous different cancers. See, e.g., Chajès et al., Cancer Research 66, no. 10: 5287-5294(May 15, 2006); Hatzivassiliou et al., Cancer Cell 8, no. 4: 311-321(October 2005); Swinnen et al., Current Opinion in Clinical Nutrition and Metabolic Care 9, no. 4: 358-365 (July 2006); Alli et al., Oncogene 24, no. 1:39-46 (Oct. 18, 2004). In adipose tissue, in contrast, levels of ChREBP β expression products are negatively correlated with disease state—i.e. reduced levels ChREBP β expression products in adipose tissue are associated with disease states such as diabetes-related disorders.

Levels of nucleic acid ChREBP β expression products can be determined by any means known in the art, including polymerase chain reaction (PCR), including reverse transcriptase (rt) PCR, real-time and quantitative PCR methods (including, e.g., TAQMAN®, molecular beacon, LIGHTUP™, SCORPION™, SIMPLEPROBES®; see, e.g., U.S. Pat. Nos. 5,538,848; 5,925,517; 6,174,670; 6,329,144; 6,326,145 and 6,635,427)); Northern blotting; Southern blotting of reverse transcription products and derivatives; array based methods, including blotted arrays or in situ-synthesized arrays; and sequencing, e.g., sequencing by synthesis, pyrosequencing, dideoxy sequencing, and sequencing by ligation, or any other methods known in the art, such as discussed in Shendure et al., Nat. Rev. Genet. 5:335-44 (2004) or Nowrousian Euk. Cell, 9(9): 1300-1310 (2010), including such specific platforms as HELICOS®, ROCHE® 454, ILLUMINA®/SOLEXA®, ABI SOLiD®, and POLONATOR® sequencing.

In particular embodiments, the invention provides oligonucleotides for detecting nucleic acid ChREBP β expression products, as well as additional tools for evaluating ChREBP β expression products—e.g., oligonucleotides to detect total (i.e. α and β) ChREBP expression products, as well as ChREBP α expression products. Based on the transcription mechanism driving nucleic acid ChREBP β expression products, in certain embodiments, the oligonucleotides provided by the invention enable detection, by any means, of exon 1b—the novel first exon of ChREBP β. The skilled artisan can readily develop a wide array of oligonucleotides to detect nucleic acid ChREBP β expression products and particular oligonucleotide pairs for detection (by amplification) of mouse (e.g. TCTGCAGATCGCGTGGAG and CTTGTCCCGGCATAGCAAC; SEQ ID NOs: 39 and 39, respectively) and human (e.g. AGCGGATTCCAGGTGAGG and TTGTTCAGGCGGATCTTGTC; SEQ ID NOs: 64 and 65, respectively) nucleic acid ChREBP β expression products are shown in TABLE 6 in the examples. Other suitable oligonucleotides for detecting nucleic acid ChREBP β expression products from human, mouse, or any other organism can readily be reduced to practice without undue experimentation. One non-limiting example of an additional modality to detect nucleic acid ChREBP β expression products includes subtractive analysis, e.g. by measuring total nucleic acid ChREBP expression product levels (α and β) and detecting nucleic acid ChREBP α expression products (e.g. by using sequence directed to the first exon of ChREBP α expression products, which is not present in nucleic acid ChREBP β expression products).

The oligonucleotides provided by the invention can readily be designed using ordinary skill in the art of molecular biology to arrive at oligonucleotides that are specific for a nucleic acid ChREBP β expression product, including, in certain embodiments, exon 1b (as well as fragments and similar nucleic acid sequences, as described above)—i.e., so that the oligonucleotides can discriminate the target nucleic acid from other nucleic acids present (or expected to be present) in a sample, including ChREBP α, entire transcriptomes and/or oligonucleotides directed to other genes.

The size of the nucleic acid ChREBP β expression product, e.g. the size of an amplified region to be detected, can be adjusted so as to provide the necessary resolution, based on the biological sample provided, e.g. the relative heterogeneity of nucleic acids in the sample or a fraction thereof. For example the nucleic acid ChREBP β expression product, or fragment thereof, detected may be at least about 10, 15, 16, 17, 18, 19, 20, 21, 22, 23, 24, 25, 26, 27, 28, 29, 30, 35, 40, 45, 50, 55, 60, 70, 80, 90, 100, 110, 120, 130, 140, 150, 160, 170, 180, 190, 200, 210, 220, 300, 400, 500, 600, 700, 800, 900, 1000, 1250, 1500, 1750, 2000, 2250, 2500, 2750, 3000, 3050, 3100 nucleotides, or more, in length and, in particular embodiments, comprises a nucleic acid that has at least 60% identity to SEQ ID NO: 1 or 5, e.g. at least 60, 65, 70, 80, 85, 90, 95, 96, 97, 98, 99 or 100% identity or which hybridizes to SEQ ID NO: 1 or 5 under highly stringent conditions.

Similarly, the length (e.g., about 10-100 nucleotides, e.g., about 10, 15, 16, 17, 18, 19, 20, 21, 22, 23, 24, 25, 26, 27, 28, 29, 30, 35, 40, 45, 50, 55, 60, 70, 80, 90, 100 nucleotides, or more) and sequence (i.e., the particular portion of a nucleic acid ChREBP β expression product to which the oligonucleotide hybridizes) of the oligonucleotides can readily be adjusted to achieve, e.g., a desired melting temperature (Tm; e.g., about 45-72° C., e.g., about 45, 50, 55, 60, 65, 70, 72° C. or more) and specificity, e.g., depending on the other materials present in the biological sample or fraction thereof. The skilled artisan will readily account for factors such as secondary structures, primer dimers, salt concentrations, nucleic acid concentrations, et cetera. Oligonucleotide primers provided by the invention may consist of (or consist essentially of) naturally occurring deoxribonucleotides or, optionally, may include modifications such as non-natural nucleotides, artificial backbones (such as PNAs), and detectable labels, such as florescent labels, biotinylation, et cetera.

Levels of protein ChREBP β expression products may be detected by any means of protein detection known in the art, including, for example, ELISA, Western Blotting, RIA (radioimmunoassay), nucleic acid-based or protein-based aptamer techniques, HPLC (high precision liquid chromatography), SPR (surface plasmon resonance), SAT (suspension array technology—including both immune-based, aptamer-based, or combination methods), direct peptide sequencing (such as Edman degradation sequencing), or mass spectrometry (such as MS/MS, optionally coupled to HPLC).

In particular embodiments, the methods provided by the invention that encompass detecting protein ChREBP β expression products employ antibodies directed to ChREBP, but which do not bind to the N-terminus of ChREBP—i.e., the portion of ChREBP that is absent in ChREBP β expression products. For example, antibodies suitable for use in detecting protein ChREBP β expression products include those that specifically bind a polypeptide having an amino acid sequence with at least 60% identity to SEQ ID NO: 2 or 6, e.g., bind a polypeptide having an amino acid sequence with at least 60, 65, 70, 80, 85, 90, 95, 96, 97, 98, 99 or 100% identity to SEQ ID NO: 2 or 6, or a fragment thereof, wherein the fragment is at least 10, 20, 30, 40, 50, 60, 70, 80, 90, 100, 150, 200, 250, 300, 350, 400, 450, 500, 550, 600, or 650 amino acids in length. In certain embodiments, subtractive or comparative methods employing multiple antibodies may be used. For example, total protein ChREBP expression products (i.e. α and β) can be measured using an antibody that binds to regions common to the α and β forms, while a second antibody that specifically binds a portion of the N-terminus of ChREBP α (e.g. within the first approximately 170-180, e.g. the first 178 amino acids of ChREBP α) not present in protein ChREBP β expression products is used to determine the proportion of total ChREBP protein that is ChREBP α, thereby providing the level of ChREBP β expression product). Similar approaches will be readily recognized by the skilled artisan based on the teachings of the application.

Levels of ChREBP β expression products can be evaluated and classified by a variety of means such as general linear model (GLM), ANOVA, regression (including logistic regression), support vector machines (SVM), linear discriminant analysis (LDA), principal component analysis (PCA), k-nearest neighbor (kNN), neural network (NN), nearest mean/centroid (NM), and baysian covariate predictor (BCP). Suitable cutoffs for evaluating levels of ChREBP β expression products (e.g., for classification as abnormal (obsese; positive or at risks for a diabetes-related disorder, cancer or vascular disease; requiring modification of a treatment regime) or normal (or low risk, or positive response to treatment) can be determined using routine methods, such as ROC (receiver operating characteristic) analysis, and may be adjusted to achieve the desired sensitivity (e.g., at least about 50, 52, 55, 57, 60, 62, 65, 67, 70, 72, 75, 77, 80, 82, 85, 87, 90, 92, 95, 97, or 99% sensitivity) and specificity (e.g., at least about 50, 52, 55, 57, 60, 62, 65, 67, 70, 72, 75, 77, 80, 82, 85, 87, 90, 92, 95, 97, or 99% specificity).

For example, in particular embodiments, expression levels are converted to a disease index. In particular embodiments, a disease index can use raw or transformed (e.g. normalized to any suitable metabolite, log-normalized, expressed as a ratio of ChREBP β and ChREBP α levels, percentile ranked, ranked as quartiles, et cetera) levels of ChREBP β expression products. A disease index for a particular individual can be compared to reference values as, for example, a percentile rank. Using percentile ranks the skilled artisan can then diagnose, prognose, or otherwise clinically stratify a subject by comparing the subject's disease index to these reference values. For example, in certain embodiments, a subject with a disease index percentile rank of at least 40, 45, 50, 60, 65, 70, 75, 80, 85, 90, or 95 may be classified as having, being at an increased risk for developing, or needing additional/alternative treatment for obesity, a diabetes-related disorder, cancer or vascular disease. In more particular embodiments, a subject exhibiting a disease index in at least the 60^{th}, e.g., at least 70^{th} or at least 75^{th} percentile is classified as having, being at an increased risk for developing, or needing additional/alternative treatment for obesity, a diabetes-related disorder, cancer or vascular disease. A selected threshold for a disease index can be set to achieve a desired sensitivity or specificity, as described above, and/or to stratify subjects based on a relative hazard ratio between stratification groups. For example, in some embodiments, a disease index threshold is set to achieve a “hazard ratio” (ratio of frequency of a disorder between two stratification groups, e.g., high and low risk of disease or complication) of about 1.1, 1.2, 1.4, 1.6, 1.8, 2.0, 2.2, 2.4, 2.5, 2.6, 2.8, 3.0, 3.5, 4.0, or more. In more particular embodiments, the index threshold is set to achieve a hazard ratio of at least about 3.8, 3.9, 4.0, 4.1, 4.2, 4.3, 4.4, or more, e.g., 5, 6, 7, 8, 9, or 10. “Stratification groups” comprises the member of a data set satisfying one or more stratification criteria—for example, a percentile rank of disease index, such as all group members with a disease index greater than or equal to the 60^{th} percentile. Stratification groups may be compared by any means by any statistic, such as, mean, median, mode, and/or standard deviation of any clinical parameter, such as age, duration of disease, frequency of death, et cetera.

Kits provided by the invention contain reagents to perform any of the methods provided by the invention, e.g. oligonucleotides and/or antibodies as described above. In particular embodiments, the kits include instructions for use. Optionally, the kits may include “suitable positive controls,” which are compositions comprising (consisting essentially of, or consisting of) nucleic acids, proteins, or nucleic acids and proteins that contain known concentrations of ChREBP β expression products and, in certain embodiments, ChREBP α expression products. For example, suitable controls may be from a clinical source known to have obesity, a diabetes-related disorder, cancer or vascular disease and may include either fixed or preserved but otherwise unprocessed biological sample, or, alternatively, isolated fractions from such samples, including fractions comprising (consisting of or consisting essentially of) nucleic acids (e.g., mRNA or cDNAs thereof), protein, and combinations thereof (e.g., at least 20, 40, 50, 60, 70, 80, 90, 95, 97, 99% by dry weight, or more, nucleic acid and/or protein). Alternatively, in certain embodiments, the suitable positive controls may comprise artificial mixtures of nucleic acids and/or proteins, e.g., combined in proportions characteristic of an abnormal levels of ChREBP β expression products for a particular disorder and/or particular ranges of concentrations.

### IV. Treatment Methods

In another aspect, the invention provides methods of treatment comprising administering a suitable prophylaxis or treatment to a subject in need thereof, as determined by the methods provided by the invention—e.g., according to any of the methods described under the previous subheading. Specifically, in certain embodiments, the subject is administered any clinically acceptable prophylaxis or treatment (including new regimens or modifications of existing regimens) for their given indication, based on the determination that the subject exhibits an abnormal level of ChREBP β expression products according to the methods provided by the invention. In more particular embodiments, any of the methods described in the claims or under the previous subheading further comprise the steps of follow-on diagnosis, prognosis or treatment. In other embodiments, the follow-on diagnosis, prognosis or treatment is performed by a provider advised of the presence of an abnormal level of a ChREBP β expression product, but who did not necessarily make the determination. For example, where the subject has been identified as having, being at an increased risk for, or needing further treatment for obesity, a diabetes-related disorder, cancer or vascular disease, at least in part, on the basis of abnormal levels of ChREBP β expression products, a provider administers or directs the subject to undergo a prophylactic and/or treatment regime suitable for the indication.

Suitable prophylaxes or treatments for diabetes-related disorders include, for example, weight loss programs, increased exercise, modified diet (e.g., reduced glycemic index), GLP-1R (human GeneID 2740) agonists (such as exenatide and liraglutide); DPP-4 antagonists (e.g., saxagliptin, vildagliptin); pramlintide; insulins (e.g., glulisine, detemir, glargine, lispro, aspart); SGLT2 (human GeneID No. 6524) inhibitors; inhibitors of glucose synthesis or release (FR-225654, CS-917 and MB07803); inhibitors of pyruvate kinase M2 (human GeneID No. 5315) (including agents described in U.S. Patent Application Publication No. 20100099726 A1, incorporated by reference in its entirety); insulin sensitizers (such as biguanidines, including metformin); adiponectin receptor 1 (human GeneID No. 51094) and adiponectin receptor 2 (human GeneID No. 79602) agonists; leptin receptor (human GeneID No. 3953) agonists; anoretics (e.g., sibutramine, rimonabant, bupropion); and the like, including combinations of the foregoing.

Suitable prophylaxes or treatments for obesity include weight loss programs, increased exercise, modified diet (e.g. reduced caloric, carbohydrate, or fat diets), gastric bypass or laproscopic banding, SGLT2 (human GeneID No. 6524) inhibitors; inhibitors of glucose synthesis or release (SB-204990, 2-deoxy-D-glucose (2DG), 3-bromopyruvate (3-BrPA, Bromopyruvic acid, or bromopyruvate), 3-BrOP, 5-thioglucose and dichloroacetic acid (DCA), FR-225654, CS-917 and MB07803); inhibitors of pyruvate kinase M2 (human GeneID No. 5315) (including agents described in U.S. Patent Application Publication No. 20100099726 A1, incorporated by reference in its entirety); leptin receptor (human GeneID No. 3953) agonists; anoretics (e.g., sibutramine, rimonabant, bupropion); and the like, including combinations of the foregoing.

Suitable prophylaxes or treatments for cancer include chemotherapy, hormonal therapy, immunotherapy, radiotherapy, surgery, targeted gene therapies (e.g., epidermal growth factor receptor-tyrosine kinase inhibitors, such as gefitinib; and agents targeting ALK mutations and rearrangements, such as crizotinib, et cetera), glycolytic inhibitors (e.g., SB-204990, 2DG, 3-BrOP, 5-thioglucose, DCA, as well as those agents described in U.S. Patent Application Publication No. 20100099726 A1), inhibitors of ATP-citrate lyase, inhibitors acetyl-CoA carboxylase, inhibitors fatty acid synthase, and combinations of the foregoing.

Suitable prophylaxes or treatments for vascular disease include weight loss programs (such as the pharmaceutical obesity treatments, above), increased exercise, modified diet (e.g. reduced salt), statin treatment (e.g. atorvastatin, fluvastatin, lovastatin, pitavastatin, pravastatin, rosuvastatin and simvastatin), bypass surgery and stenting.

It is, of course, encompassed by the methods provided by the invention that a subject determined to have obesity, a diabetes-related disorder, cancer or vascular disease may have one or more of these disorders, e.g. 2, 3 or 4 of these disorders, and may therefore provide an indication for treatment with combinations of prophylaxes or treatments for the different disorders, i.e. combinations of prophylaxes or treatments for obesity, a diabetes-related disorder, cancer or vascular disease as described above.

In another aspect, the invention provides methods of modulating the activity of ChREBP β expression products, i.e. increasing or decreasing their activity. Such methods can be performed in vitro or in vivo, e.g. in methods of treatment. For example, to increase the activity of ChREBP β expression products, an agonists of ChREBP β expression products can be contacted with a target cell, e.g., within a subject. This contact can be any means including by, e.g., intra venous, or intra peritoneal, rectal, oral or nasal administration of a recombinant polypeptide corresponding to a protein ChREBP β expression product, but which does not comprise a functional “LID” domain, as depicted in FIG. 18 and described further in Li et al., 2006, or by contacting the cell with a nucleic acid encoding said polypeptide, including, without limitation a recombinant cell containing said nucleic acid, e.g., in plasmid form or as a stable chromosomal construct. In certain embodiments, the agonist of ChREBP β expression product may be a proteasome inhibitor, since ChREBP β is degraded in a proteasome dependent manner but ChREBP α is not. In some embodiments, the agonist of ChREBP β expression products may be directed to adipose tissue.

Similarly, antagonists of ChREBP β expression products may be used in methods of treatment where reduced levels of ChREBP β expression products are desireable, e.g., in cancer, or, for diabetes-related disorders, elevated ChREBP β expression products in the liver. Suitable antagonists of ChREBP β expression products include siRNA directed to a nucleic acid ChREBP β expression products, e.g., in certain embodiments the siRNA is directed to exon 1b of a nucleic acid ChREBP β expression product. In other embodiments, the antagonist may be a dominant negative recombinant polypeptide corresponding to a ChREBP β expression product—i.e., that does not include a functional “LID” domain, as depicted in FIG. 18 and described further in Li et al., 2006—as either a purified polypeptide or a nucleic acid encoding the polypeptide, including a recombinant cell comprising such a nucleic acid—either in plasmid or stable chromosomal form. Exemplary dominant negative forms of ChREBP based on ChREBP β include inactivation of its DNA binding domain, e.g., by deletion or mutation to a non-functional form, which is depicted in FIG. 18—i.e. the bHLH/ZIP domain. Additional dominant negative proteins for inhibiting ChREBP β expression product activity includes dominant negative forms of Mlx (human GeneID No. 6945), including those that lack a functional DNA-binding domain (e.g., by deletion or mutation—for examples, see, e.g., Ma et al., J. Biol. Chem. 280:12019-27(2005), which is incorporated by reference in its entirety).

### V. Screening Methods

In another aspect, the invention provides screening methods for identifying an agent that modulates the expression of a ChREBP β target gene, as defined above. The methods comprise determining the level of a ChREBP β expression product reporter in a cell contacted with a candidate agent where a change in the level of the ChREBP β expression product reporter in the cell relative to a control cell not contacted with the agent indicates that the agent modulates the expression of a ChREBP β target gene.

Any cell type may be useful in such methods including, yeast or insect cells, or a mammalian cell, e.g. a primate, murine, bovine, ovine, leporine, or porcine cell. The cell may be isolated, e.g., the method is performed in vitro, in a suitable culture, e.g., with either primary or established cell lines. In some embodiments, the cells are recombinant while in other embodiments the cells are non-recombinant. In other embodiments, the cell is in situ, i.e., the method is performed in vivo in a non-human animal, and in more particular embodiments in a non-human mammal, such as a non-human primate, a leporine or murine animal. The non-human animal may be transgenic or non-transgenic and such animals may also serve as the source of cells for the in vitro screening methods provided by the invention. The cell, in certain embodiments, further expresses Mlx. In other embodiments, the cell further express GLUT4 (SLC2A4, human GeneID No. 6517) and in more particular embodiments, the cell further expresses both Mlx and GLUT4. Although not essential to all aspects of these screening methods, in some embodiments, the methods include incubating the cell in the presence of glucose or fructose or their metabolites or analogs such as glucose-6-phosphate, xyulose-5-phosphate, fructose-2,6-bisphosphate, mannoheptulose, 2-deoxyglucose (2DG), or fluorodeoxyglucose.

The screening methods provided by the invention can, in some embodiments, include screening the candidate agent in the presence of additional agents, e.g., a second (or more) candidate therapeutic. For example, second therapeutic agents can include one or more of any of the therapeutics described under the previous subheading in order to identify combinations of agents with synergistic interactions.

In some embodiments, the ChREBP β expression product reporter is a ChREBP β target gene (i.e., a non-recombinant, endogenous product). In other embodiments, the ChREBP β expression product reporter is a synthetic (e.g. recombinant) ChREBP β expression product reporter and in more particular embodiments the synthetic ChREBP β expression product reporter is an expression product of a nucleic acid construct comprising a ChREBP β promoter, e.g., comprises a sequence having at least 60% identity to SEQ ID NO: 1 or 5. In still more particular embodiments, the construct comprises a nucleic acid sequence that has at least 60% identity to SEQ ID NO: 3 in operative association with a heterologous sequence. Exemplary heterologous sequences useful in these methods include, for example, a fluorescent protein, luciferase, aequorin, a peptide epitope, or an enzyme. Accordingly, in another aspect, the invention provides isolated nucleic acids encoding any of the foregoing reporter constructs.

Agents identified by these screening methods may either increase or decrease the expression of a ChREBP β target gene and/or increase or decrease the activity of a ChREBP β expression product. Methods of treating obesity, a diabetes-related disorder, cancer or vascular disease by administering an effective amount of one or more of the agents identified by these screening methods are contemplated and encompassed by the present invention.

The following examples serve to illustrate, and in no way to limit, the present invention.

### EXEMPLIFICATION

Insulin resistance is a common complication of obesity and is a major factor in the pathogenesis of type 2 diabetes and cardiovascular disease¹. Adipose tissue contributes to the development of obesity-related insulin resistance through increased release of fatty acids, altered adipokine secretion, and/or macrophage infiltration and cytokine release^{2,3}. Altered adipose tissue glucose metabolism is also an important cause of insulin resistance, and adipose tissue Glut4 (Slc2a4), the major insulin-responsive glucose transporter, plays a more central role in systemic glucose metabolism than previously appreciated^{1,4,5}. In insulin-resistant states, Glut4 is down-regulated in adipose tissue, but not in muscle¹, the major site of insulin-stimulated glucose uptake. In addition, mice with adipose-specific Glut4 overexpression (AG4OX) have improved glucose homeostasis⁵ while adipose-specific Glut4 knockout mice (AG4KO) have insulin resistance and type 2 diabetes⁴. We investigated how altering adipose tissue glucose flux regulates glucose homeostasis. Here we show that carbohydrate responsive-element binding protein (ChREBP, also known as Mlxipl), which is a glucose-responsive transcription factor that regulates fatty acid synthesis and glycolysis,⁶ is highly regulated by Glut4 in adipose tissue and is a key determinant of systemic insulin sensitivity and glucose homeostasis. We also demonstrate that ChREBP in adipose tissue is required for the improved glucose homeostasis resulting from increased adipose-Glut4 expression. Glut4-mediated glucose uptake induces ChREBP which activates adipose tissue de novo lipogenesis. The latter is associated with enhanced insulin sensitivity⁷⁻¹⁰. We also show in obese humans that adipose-ChREBP gene expression correlates with insulin sensitivity, suggesting that ChREBP protects against obesity-associated insulin resistance. In addition, we discovered a novel mechanism for glucose-regulated ChREBP expression which involves a new ChREBP isoform, ChREBPβ. ChREBPβ is expressed from an alternative promoter in a glucose- and ChREBP-dependent manner. In contrast, the expression of the canonical ChREBPα isoform is not regulated by glucose flux. However, glucose-induced ChREBPα transcriptional activity increases ChREBPβ expression. Furthermore, expression of ChREBPβ is more highly regulated than ChREBα in adipose tissue in insulin resistant states. Thus, activation of adipose tissue ChREBP, and particularly ChREBPβ, may be a novel strategy for preventing and treating obesity-related metabolic dysfunction and type 2 diabetes.

### Methods Summary:

AG4OX, AG4KO, and ChREBP KO mice were described previously⁴⁻⁶ except that AG4KO mice were generated using adiponectin-Cre expressing mice⁴³ rather than aP2-Cre expressing mice. Phenotypic analyses were performed as previously described^{4,5,44}. All mouse studies were conducted in accordance with federal guidelines and were approved by the BIDMC Institutional Animal Care and Use Committee. For microarray analysis, RNA was isolated from epididymal adipose tissue and the cDNA was analyzed on the Affymetrix MG-U74-A.v2 Genechip. Quantitative real-time PCR was performed on the Applied Biosystems 7900 HT using SYBR Green PCR Master Mix. Cloning, mutagenesis, and luciferase assays were performed by standard methods. Human subjects were recruited as previously described^{41,45}. Human studies were approved by the Human Studies Committee of Washington University School of Medicine or the Ethics Committee of the University of Leipzig.

### Methods:

### Animal Studies:

Generation and initial metabolic characterization of the adipose-specific Glut4-overexpressing mice (AG4OX), adipose-specific Glut4 knock-out mice (AG4KO), and ChREBP KO mice were previously described⁴⁻⁶ except that AG4KO mice were generated using adiponectin-Cre expressing mice⁴³ rather than aP2-Cre expressing mice. WT and adiponectin-Cre littermates were used as controls for AG4KO mice. Mice were housed at Beth Israel Deaconess Medical Center with a 14/10 light-dark cycle and were fed standard chow (Formulab 5008) or HFD (Harlan-Teklad TD.93075) ad libitum. All studies were performed on age- and sex-matched littermates. Blood collections were performed by submandibular vein or tail vein bleeding. Body composition was measured by NMR (Echo Medical Systems). Glucose tolerance tests were performed by injection of 1 mg glucose per kg body weight i.p. after 5 hours food removal. For the fasting glycemic time course experiment, food was removed at 8 AM. Mice were sacrificed by CO2 euthanasia or decapitation and serum was collected, and tissues were harvested, snap frozen in liquid nitrogen, and stored at −80 C for processing. Mouse studies were conducted in accordance with federal guidelines and were approved by the BIDMC Institutional Animal Care and Use Committee.

### Human Studies:

For the cross-sectional cohort, adipose tissue samples were obtained from 123 men (n=64) and women (n=59) who underwent open abdominal surgery for gastric banding, cholecystectomy, appendectomy, weight reduction surgery, abdominal injuries or explorative laparotomy. These patients represent a normal glucose tolerant subset of patients previously described⁴⁵. All subjects had a stable weight, defined as the absence of fluctuations of >2% of body weight for at least 3 months before surgery. Patients with malignant diseases or any acute or chronic inflammatory disease, as determined by a leukocyte count of >7,000 Gpt/l, C-reactive protein levels of >50 mg/l or clinical signs of infection, were excluded from the study. After resection, samples of visceral and subcutaneous adipose tissue were immediately frozen in liquid nitrogen. Euglycemic-hyperinsulinemic clamp studies were performed prior to surgery in subjects undergoing bariatric surgery or 3 months after surgery in subjects who underwent non-elective surgeries. The clamp study was performed with an insulin infusion rate of 20 mU/kg/min, as described and the glucose disposal rate was defined as the glucose infusion rate during the last 30 min of the study⁴⁵. The study was approved by the ethics committee of the University of Leipzig. All participants gave written informed consent before taking part in the study.

For the obese cohort, 38 obese subjects (10 men, 28 women; 41±11 years old) who had normal oral glucose tolerance were studied. Subjects were sedentary (i.e., participated in regular exercise <1 hour/wk and ≦1 time/wk) and weight stable. All subjects provided written, informed consent before participating in the study, which was approved by the Human Studies Committee of Washington University School of Medicine in St. Louis.

Subjects were admitted to the Clinical Research Unit at Washington University School of Medicine in the evening before the clamp procedure. The next morning, after subjects fasted for 12 h overnight, a hyperinsulinemic-euglycemic clamp procedure in conjunction with [6,6-²H₂]glucose tracer infusion was performed, as described previously⁴¹. Euglycemia was maintained at a blood glucose concentration of approximately 5.6 mmol/L (100 mg/dL) by infusing 20% dextrose enriched to 2.5% with [6,6-²H₂]glucose. Skeletal muscle insulin sensitivity was determined as the relative increase in the rate of glucose uptake during insulin infusion (50 mU/m²/min).

### Microarray Analysis:

Total RNA from epididymal adipose tissue was extracted using the RNeasy Mini Kit from Qiagen from three mice from each of four genotypes: aP2-Cre transgenic littermates (controls for AG4KO mice), AG4KO mice; FVB littermates (controls for AG4OX) and AG4OX. RNA from each mouse was hybridized on an Affymetrix MG-U74-A.v2 Genechip microarray. Affymetrix gene chip hybridization and analysis were performed at the Genomics Core Facility of the Beth Israel Deaconess Medical Center. Genome-wide expression analysis of the microarray data were performed using gene set enrichment analysis (GSEA)¹¹ and the s2.mgu74av2.gmt gene set database available through the Broad Institute.

### Gene Expression Analysis:

Mouse tissues were harvested following CO2 euthanasia or decapitation, snap frozen in liquid nitrogen, and stored at −80 C for processing. Total RNA was extracted from frozen tissue with TRI Reagent (Molecular Research Center, Inc.). Reverse transcription was performed using the Advantage RT-for-PCR kit (Clontech). Quantitative PCR was performed using SYBR Green PCR Master Mix (Applied Biosystems) in a 7900 HT thermocycler (Applied Biosystems). SDS 2.3 (Applied Biosystems) was used for calculation of cycle thresholds. Relative expression levels were determined using the standard curve method with normalization of target gene expression levels to 18 s or Tbp. See Table 6 for primer sequences.

For the cross-sectional cohort, total RNA was isolated from subcutaneous adipose tissue samples using TRIzol (Life Technologies) and cDNA was synthesized with standard reagents (Life Technologies). Quantitative PCR was performed using Brilliant SYBR Green QPCR Core Reagent Kit (Stratagene) in a PRISM 7900 thermocycler (Applied Biosystems). Relative expression levels were determined using the standard curve method with normalization of target gene expression levels to 18 s.

For the obese cohort, total RNA was isolated from subcutaneous adipose tissue samples using TRIzol (Invitrogen) and cDNA was synthesized using Taqman Reverse Transcription (Applied Biosystems). Quantitative PCR was performed using SYBR Green PCR Master Mix (Applied Biosystems) on the ABI 7500 Real-Time PCR System (Applied Biosystems). Relative expression levels were determined using the 2^{-ΔCt} method with normalization of target gene expression levels to the 36B4 gene.

### Western Blotting:

For adipose tissue lysates, aliquots of frozen adipose tissue were homogenized on ice in RIPA buffer supplemented with phospho-preserving and anti-protease agents (sodium fluoride, sodium pyrophosphate, sodium orthovanadate, PMSF, aprotinin, and leupeptin). For adipose tissue nuclear lysates, an adipose tissue sample was ground under liquid nitrogen, then dounce homogenized in low salt buffer [potassium chloride, 10 mM; Hepes pH 7.9, 10 mM; EDTA 0.1 mM; EGTA 0.1 mM; DTT 1 mM] with protease inhibitors. Homogenates were centrifuged at 1000 RCF for 10 minutes at 4 C and nuclear pellets were collected and washed with low salt buffer. The nuclear pellets were resuspended in RIPA buffer and vortexed vigorously. Nuclear lysates from 3 to 4 animals from the same genotype were pooled for further processing. Protein concentrations were assayed using the BCA assay. Equal amounts of protein were loaded and transferred to nitrocellulose membranes. Membranes were probed with antibodies against Acetyl-CoA Carboxylase (Cell Signaling), Fatty Acid Synthase (Cell Signaling), Glut4 (provided by H. Haspel), SREBP1 (Novus Biologicals), and Lamin B (Santa Cruz) and PI3 kinase p85 (Upstate). Film images were scanned (Epson Expression 10000 XL) and results were quantified with ImageQuant TL software (GE).

### In Vivo Fatty Acid Synthesis:

Conscious, freely moving mice in the fed state were injected intraperitoneally with 5 mCi of ³H₂O (MP Biomedicals) and 10 μCi of [U-¹⁴C]-glucose (Perkin Elmer) and sacrificed 1 hour later using an overdose of ketamine and xylazine (160 and 24 mg/kg i.p., respectively). Samples of plasma were obtained serially at 5, 10, 30, and 60 minutes after injection for measurement of the plasma glucose levels and the specific activity of plasma water and glucose. At sacrifice, tissues were rapidly removed, frozen in liquid N₂ and stored at −80° C. for processing. Lipids were extracted by the Folch method with chloroform/methanol (2:1). Fatty acids were obtained by saponification and extraction with petroleum ether. Incorporation of ³H and ¹⁴C into saponified fatty acids were measured. The rate of synthesis was calculated as a molar rate based on the estimate that 13.3 mol of H₂O are incorporated into each newly synthesized C16 fatty acid⁴⁶. The rate of glucose incorporation into newly synthesized fatty acids was calculated as nanomoles of 14C-glucose incorporated into fatty acids/gram tissue per minute, using ¹⁴C counts in saponified fatty acids extracted from tissue normalized by the time averaged specific activity of glucose in plasma over the course of the hour-long experiment.

### Glucose Uptake in Isolated Adipocytes:

Adipocytes were isolated from perigonadal fat pads and glucose uptake was measured as previously described⁵. Briefly, perigonadal fat pads were digested with collagenase (1 mg/ml) and cells were incubated at 37 “C with constant shaking in Krebs-Ringer-Hepes (30 mM) buffer (pH 7.4) with 2% bovine serum albumin, 200 nM adenosine, and without (basal) or with (insulin-stimulated) 80 nM insulin. Following a 30-min incubation with or without insulin, U-¹⁴C glucose (3 μM) was added for 60 min and the reaction was terminated by separating cells from media by spinning the suspension through dinonyl-phthalate oil. A portion of isolated adipocytes from each sample were fixed with osmic acid and counted in a Coulter counter to normalize glucose uptake per cell.

### Identification of ChoRE and ChREBP Exon 1b:

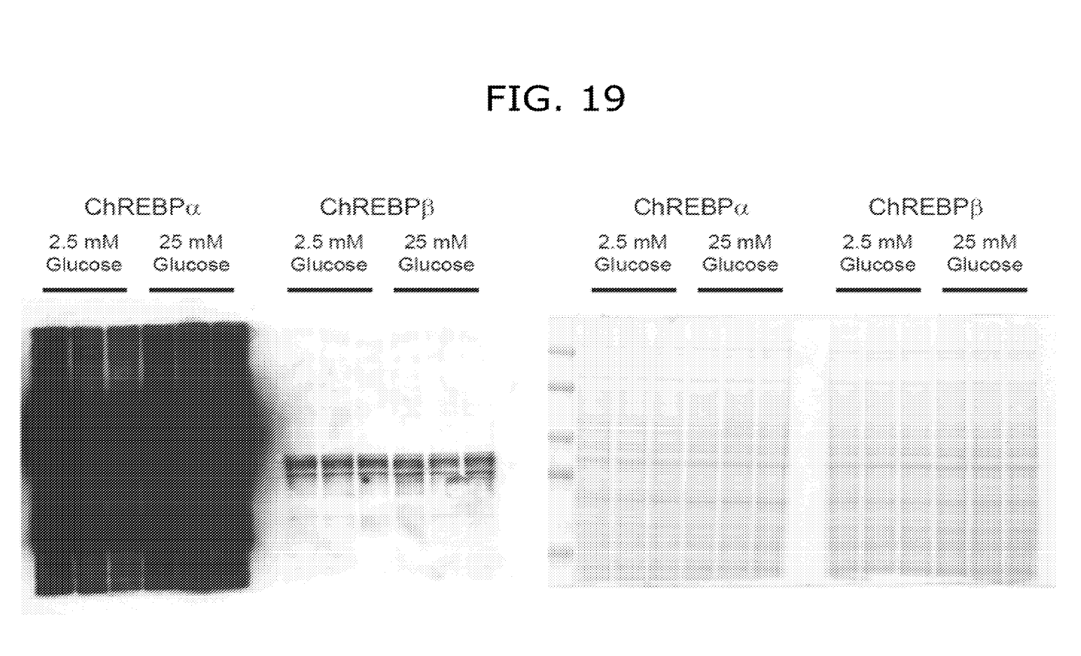

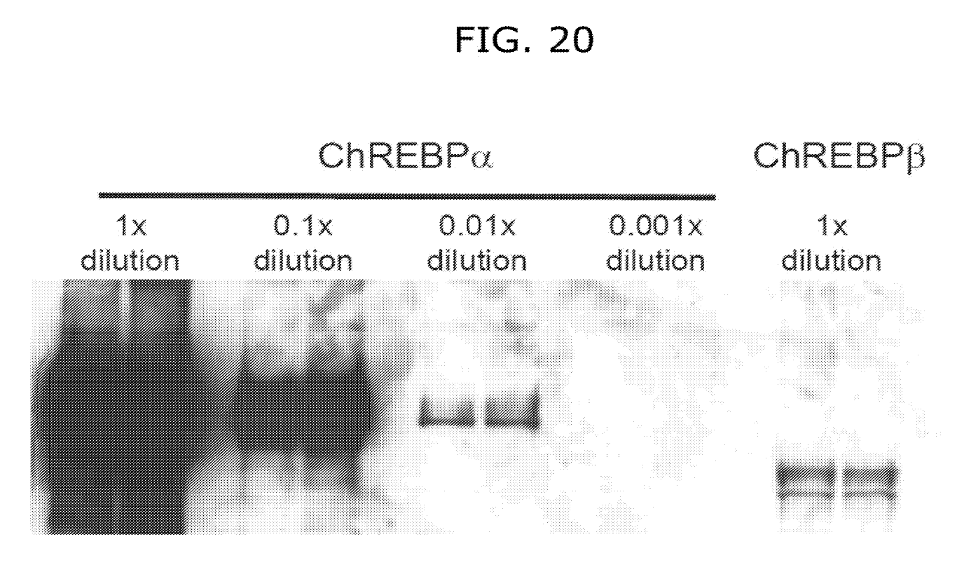

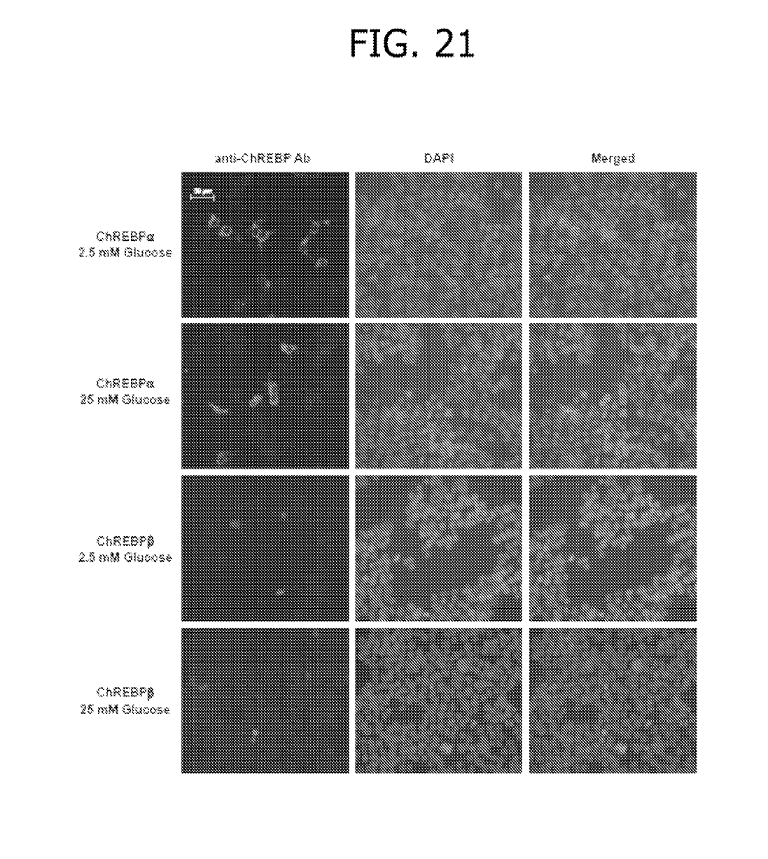

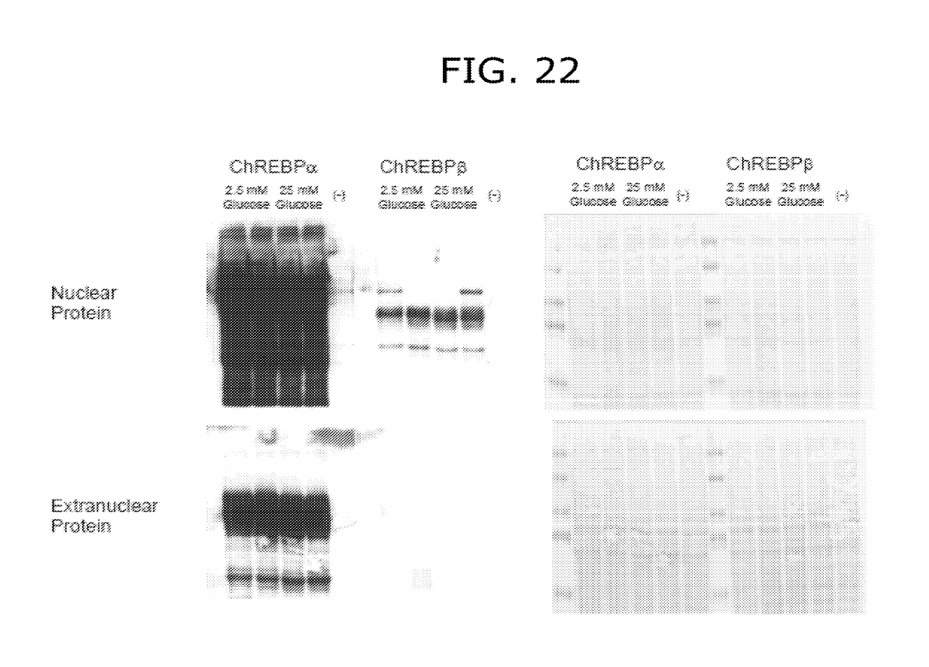

A position weighted matrix representative of a consensus ChoRE was generated from sequences for 16 mouse, human, or rat ChoREs previously identified and experimentally validated (FIG. 19). Genomic sequence 20 kb upstream and downstream of the mouse ChREBP transcriptional start site was obtained from the UCSC Genome Browser (Mouse July 2007 (NCBI37/mm9) Assembly)^{47,48} and was scanned using the Transcriptional Element Search System (www.cbil.upenn.edu/tess). Genomic sequence in the exon 1b region was obtained through the UCSC genome browser and comparisons across species were performed with Megalign (Dnastar) using the Clustal W algorithm. Analysis of histone methylation marks was performed using the Encode browser and database^{49,50}. ChREBP exon 1b was cloned from RNA prepared from AG4OX adipose tissue by 5′ rapid amplification of cDNA ends (GeneRacer, Invitrogen). A full-length ChREBPβ mRNA species was cloned using GeneRacer reagents. All cloned products were verified by sequencing.

### Functional Analysis of ChoREs:

PCR of bacterial artificial chromosome containing the mouse ChREBP gene (CH29-535J6, Children's Hospital Oakland Research Institute) was used to generate the mouse ChREBPβ promoter sequence (−631 to +224, in reference to exon 1b transcriptional start site) which was cloned into the pGL3_basic reporter vector. The putative ChoRE sequence (+157 to +174) and E-box (−98 to −92) were deleted by site-directed mutagenesis (QuickChange II Site-Directed Mutagenesis Kit, Agilent). The exon 1b promoter_pGL3, mutant pGL3 constructs, or an empty pGL3_basic control were co-transfected using Lipofectamine LTX (Invitrogen) in HEK293T cells with Flag-tagged ChREBPα (ζ-isoform) and HA-tagged Mlx, both in the CMV4 vector³³, along with a renilla luciferase control. After 24 hours of transfection, cells were cultured in DMEM containing low (2.5 mM) versus high (25 mM) glucose. Cells were collected and firefly luciferase activity was measured 24 hours after the media change and normalized to renilla luciferase activity (Dual Luciferase Assay System, Promega). All experiments were repeated at least three times with comparable results.

### Functional Analysis of ChREBPβ:

Flag-tagged ChREBPβ was generated by deletion of N-terminal sequence in the mouse Flag-tagged ChREBPα (GenBank: AF245475) in the CMV4 vector³³ by site-directed mutagenesis (QuickChange II Site-Directed Mutagenesis Kit, Agilent). ChREBPβ sequence was confirmed by sequencing. Flag-tagged ChREBPα, Flag-tagged ChREBPβ, or empty pGL3_basic control were co-transfected with HA-tagged Mlx and a reporter plasmid containing a promoter region consisting of two copies of the ACC ChoRE fused to the firefly luciferase gene⁴⁰. 24 hours after transfection, cells were cultured in DMEM containing low (2.5 mM) or high (25 mM) glucose. Cells were collected and firefly luciferase activity was measured 24 hours after the media change and normalized to renilla luciferase activity (Dual Luciferase Assay System, Promega). All experiments were repeated at least three times with comparable results.

### Statistics:

All values are given as means±S.E. Differences between two groups were assessed using unpaired two-tailed Student's t-tests unless otherwise specified in figure legends. Comparisons between multiple groups were performed by ANOVA with pairwise comparisons and adjustment for multiple comparisons by the Tukey test unless otherwise specified in figure legends. For analyses with non-normal distributions or unequal variances, ANOVA and pairwise comparisons were performed on log-transformed values. All data presented in the figures are of non-transformed values. Statistical Analyses including linear regressions were performed with SPSS 8.0.

### Results

### Glucose Regulates Adipose Tissue ChREBP

To understand the cellular mechanisms by which adipocytes respond to changes in glucose flux, we undertook global gene expression analyses of adipose tissue from AG4OX and AG4KO mice. Gene-set enrichment analysis¹¹ demonstrated coordinate up-regulation of enzymes of fatty acid synthesis in AG4OX mice (Table 1). We confirmed that in adipose tissue, the expression of the enzymes of fatty acid synthesis are coordinately up-regulated in AG4OX mice and down-regulated in AG4KO mice (FIG. 1a). Therefore we investigated the expression of transcription factors known to regulate lipogenic enzymes, e.g. sterol regulatory element binding protein 1c (SREBP-1c) and ChREBP^{6,12}. Expression of ChREBP but not SREBP-1c was increased 50% in AG4OX and decreased by 44% in AG4KO adipose tissue compared to littermate controls (FIG. 1b). SREBP-1c transcriptional activity was primarily determined by the accumulation of mature SREBP-1c in the nucleus¹³. However, the nuclear abundance of SREBP-1c was not increased in AG4OX adipose tissue (FIG. 1c). Liver X receptors alpha and beta (LXRα and LXRβ) regulate the expression of both ChREBP and SREBP-1c^{14,15}, and can coordinately regulate the expression of fatty acid synthetic enzymes independent of ChREBP and SREBP-1c¹⁶. Moreover, LXRs may act as glucose sensors¹⁷, but in the liver, glucose-mediated activation of ChREBP does not require LXR¹⁸. Expression of Abca1 and Idol, canonical LXR targets, did not change (FIG. 1d) in AG4OX or AG4KO adipose tissue indicating that LXR activity was unchanged and is not driving the changes in ChREBP or lipogenic enzyme expression. In contrast, expression of RGS16 and Txnip, two ChREBP transcriptional targets¹⁹⁻²¹ not known to be regulated by other lipogenic transcription factors, were reciprocally regulated in AG4OX and AG4KO mice (FIG. 1e) supporting a role for ChREBP in mediating the changes in lipogenic gene expression as a result of genetic changes in Glut4 expression and glucose flux into adipocytes.

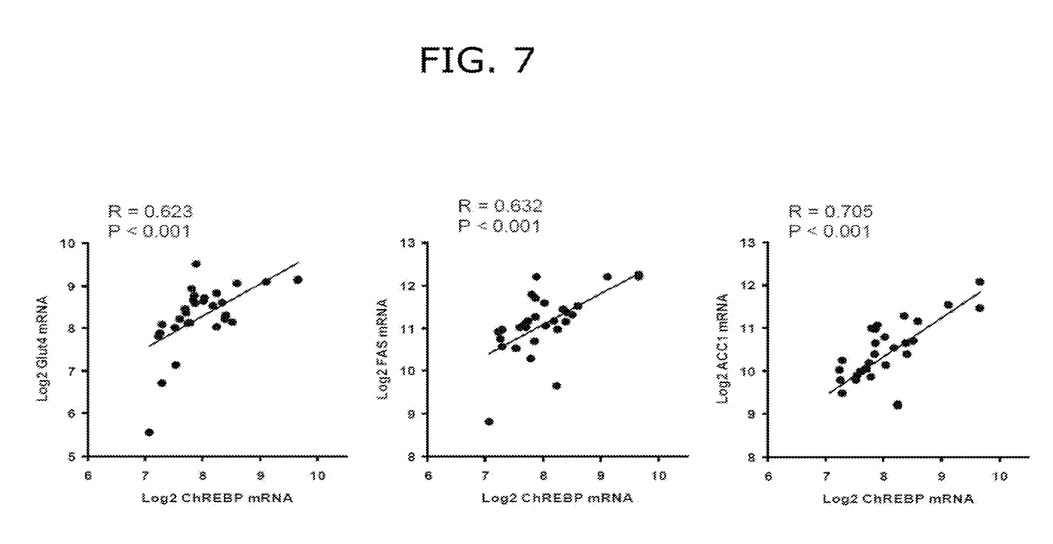

In AG4KO and control mice, adipose-ChREBP expression strongly correlated with Glut4 expression (FIG. 1f) and this is also seen across 30 mouse strains (FIG. 7)^{22,23}. The expression of ChREBP transcriptional targets FAS and ACC1 also strongly correlated with ChREBP expression across these different genetic strains (FIG. 7). Together, these data suggested that adipose tissue ChREBP may mediate the effects of changes in Glut4 expression and glucose flux on lipogenic enzyme expression.

### ChREBP Mediates the Effects of Altered Adipose Tissue Glucose Flux

Increased fatty-acid synthesis in rodent adipose tissue is associated with enhanced glucose tolerance and insulin sensitivity^{24,25}. Consistent with the increases in ChREBP and lipogenic enzyme expression (FIG. 1a-b), fatty acid synthesis rates in vivo were increased in perigonadal (8-fold) and subcutaneous (14-fold) adipose tissue in AG4OX mice compared to wild-type (FIG. 2a).

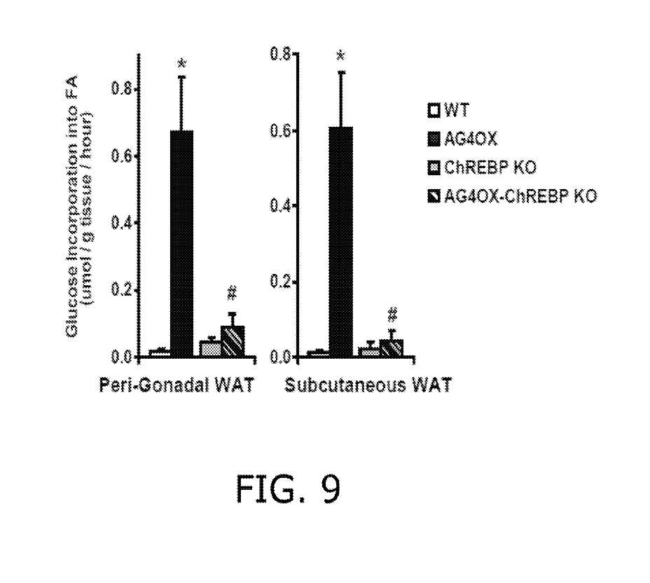

To determine whether adipose tissue ChREBP was required for the increased adipose tissue fatty-acid synthesis resulting from Glut4 overexpression, we crossbred AG4OX mice with whole-body ChREBP KO mice⁶. Fatty acid synthesis was normalized in perigonadal and subcutaneous adipose tissue after deletion of ChREBP in AG4OX mice (FIG. 2a). Glucose incorporation into newly synthesized fatty acids was also decreased in WT and AG4OX adipose following ChREBP knockout (FIG. 9). In liver, fatty acid synthesis was normal in AG4OX and AG4OX-ChREBP KO mice and was not decreased in ChREBP KO (FIG. 2a) in contrast to data from a prior report⁶. This difference may be attributable to significant differences in age, sex, genetic background and/or diet.

In parallel with the changes in lipogenesis, the marked increases in the expression of fatty acid synthetic enzymes in AG4OX adipose tissue normalize following deletion of ChREBP (FIG. 2b). This was accompanied by corresponding changes in fatty acid synthase and acetyl-CoA carboxylase protein levels (FIG. 2c). Thus, we determined that ChREBP is a major regulator of the fatty acid synthetic enzymes in adipose tissue and mediates the changes in lipogenic enzyme expression resulting from increased glucose flux.

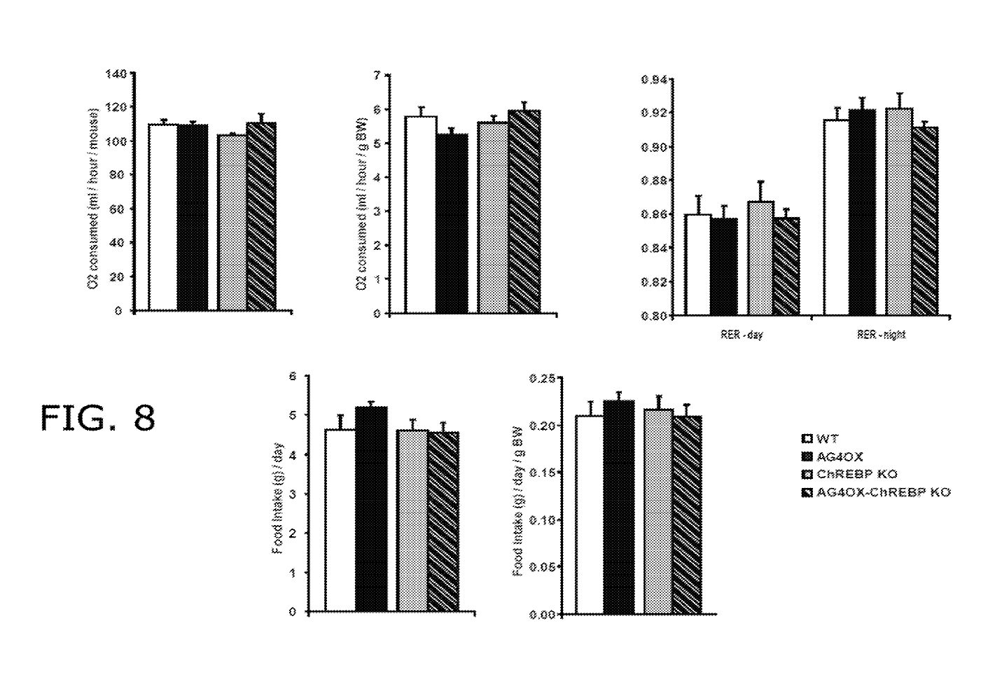

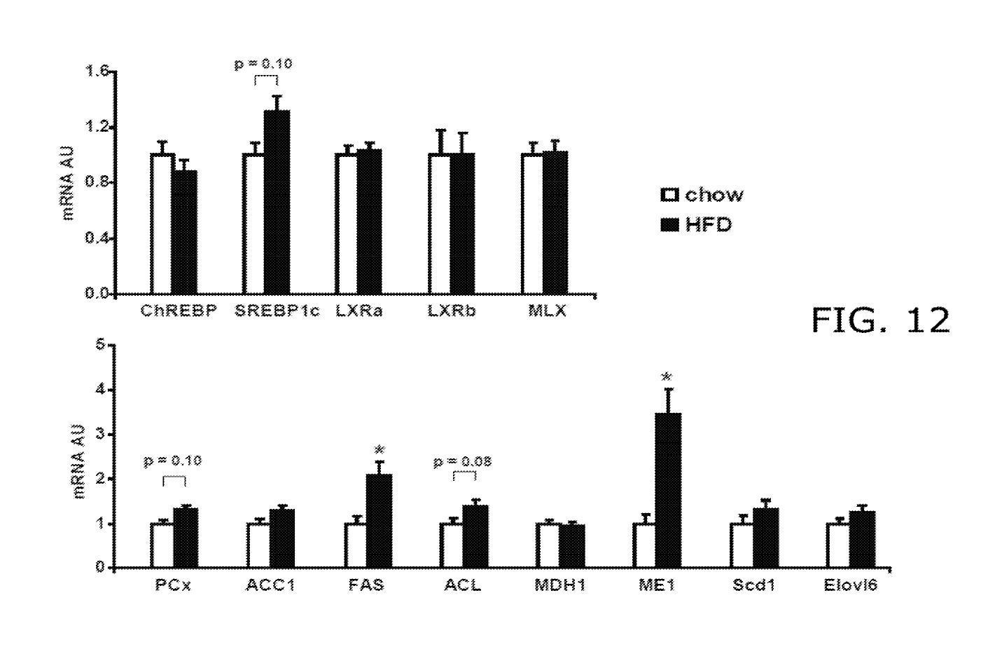

AG4OX mice were obese compared to wild-type mice (FIG. 2d-e) but had enhanced glucose tolerance (FIG. 2f)⁵. The increased body weight (FIG. 2d) and adiposity (FIG. 2e) normalized with deletion of ChREBP. A tendency toward increased food intake also normalized with ChREBP KO and no difference in energy expenditure was detected compared to WT mice (FIG. 8). In spite of the obesity, AG4OX mice were more insulin-sensitive than WT mice as evidenced by lower fed glucose levels, a trend towards lower fed insulin levels and a lower glucose-insulin product (Table 2). Decreased fed glycemia (Table 2), the markedly enhanced glucose tolerance (FIG. 2f), and the lower glycemia during insulin tolerance testing (FIG. 2g) in AG4OX mice normalized after deletion of ChREBP. Genetically ablating ChREBP in WT and AG4OX mice impairs insulin sensitivity as demonstrated by increased fed insulin levels and insulin-glucose product (P≦0.01, 2-way ANOVA) (Table 2). We observed modest glucose intolerance in ChREBP KO mice compared to WT mice and AG4OX-ChREBP KO mice (FIG. 2f) consistent with previous reports⁶. Similarly, glycemia in ChREBP KO mice remained elevated following food removal compared to WT mice (FIG. 2h). ChREBP KO prevented the relative hypoglycemia which develops in AG4OX mice within five hours of food removal (FIG. 2h). Knocking out ChREBP did not decrease the highly elevated Glut4 expression (FIG. 2i) or basal or insulin-stimulated glucose uptake (FIG. 2j) in AG4OX adipocytes indicating that the mechanism by which ChREBP regulates glucose homeostasis was not simply by altering adipose tissue glucose uptake. The decreased insulin sensitivity and glucose tolerance in AG4OX-ChREBP KO mice was not due to elevated serum triglycerides or non-esterified fatty acids since both metabolites decreased compared to AG4OX mice (Table 2). The effects of ChREBP KO on metabolism and gene expression in AG4OX mice appeared to result from the absence of ChREBP specifically in adipose tissue because ChREBP expression was increased in adipose tissue (FIG. 1b) but not in liver of AG4OX mice (FIGS. 10 and 12). The reversal of enhanced glucose tolerance and insulin sensitivity with normalization of fatty acid synthesis is consistent with a role for adipose tissue fatty acid synthesis in improving systemic glucose metabolism.

### Obesity Regulates Adipose Tissue ChREBP

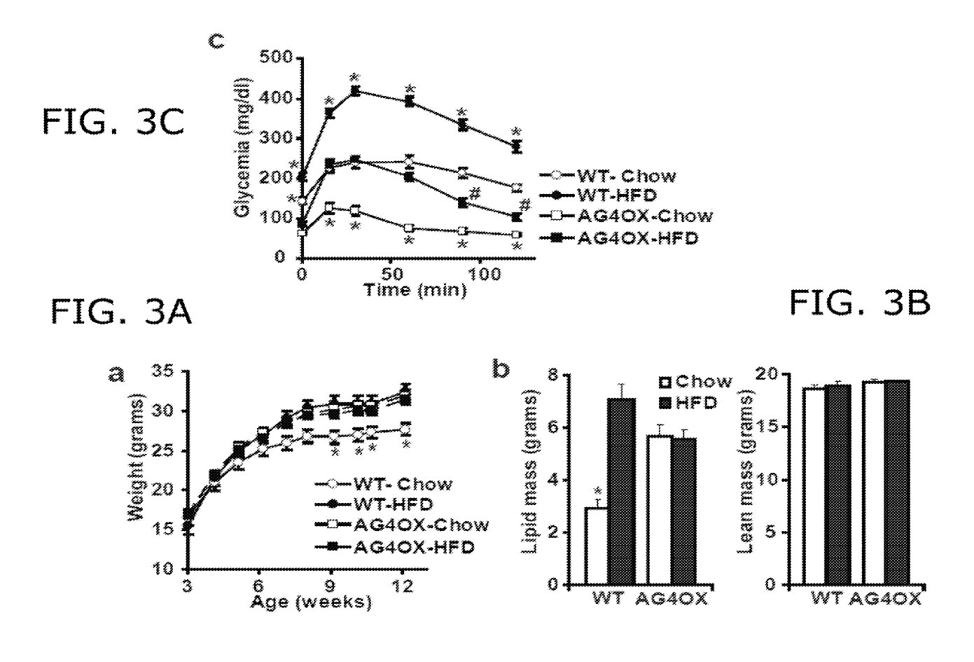

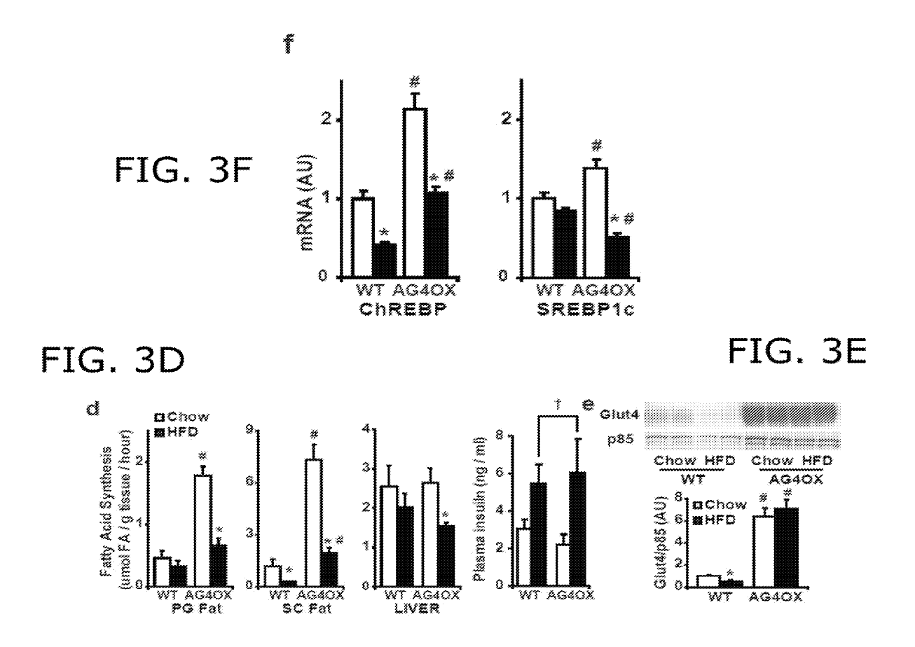

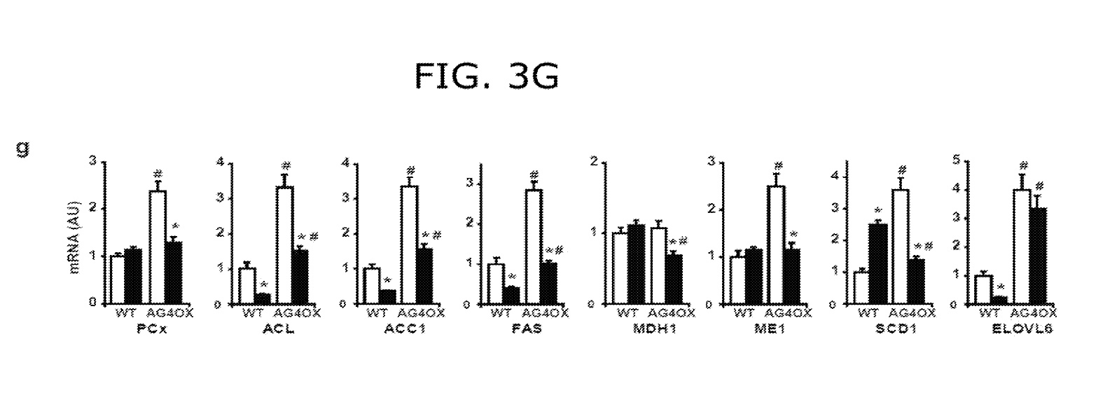

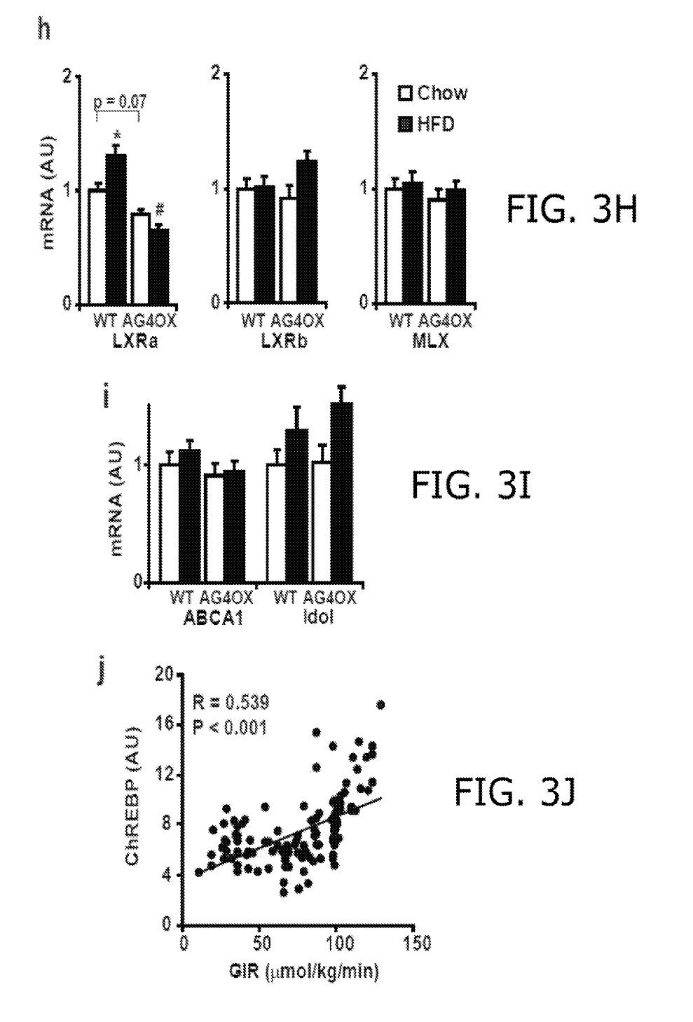

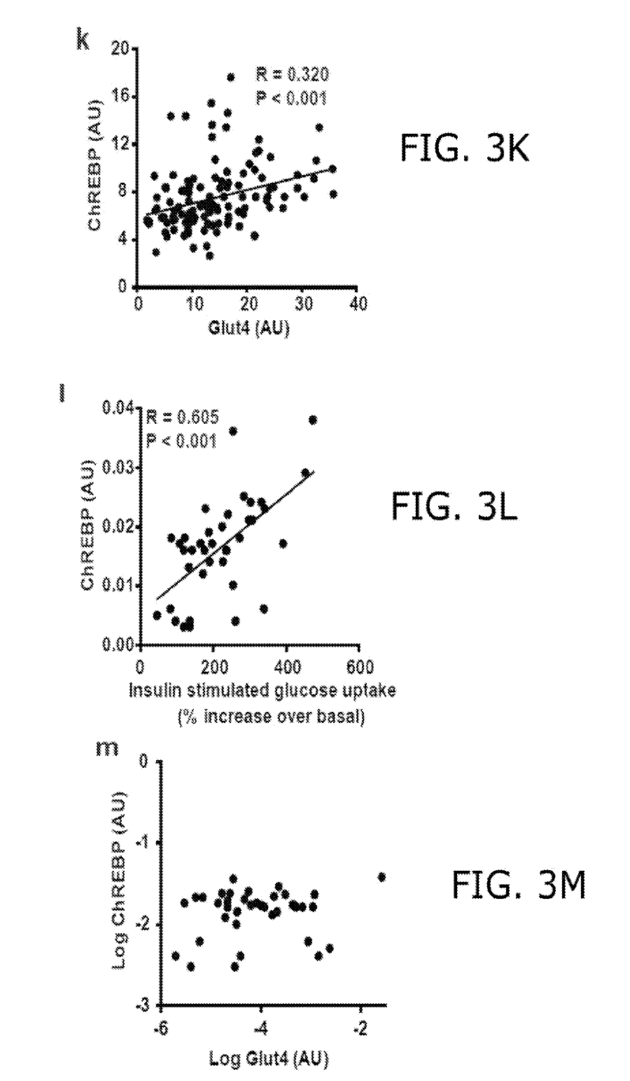

High-fat feeding causes obesity and insulin resistance in humans and rodents and down-regulates Glut4 expression selectively in adipose tissue¹. To investigate whether adipose ChREBP expression may play a role in the insulin resistance that results from down-regulation of adipose-Glut4 expression with high-fat feeding, we subjected wild-type and AG4OX mice to high-fat diet (HFD). On chow, AG4OX mice were obese compared to chow-fed wild-type mice (FIGS. 2a-b and 3a-b). AG4OX did not gain more weight on HFD and their degree of obesity on both diets was similar to HFD-fed wild-type (FIG. 3b). Lean mass was normal in AG4OX on both diets.

HFD induced insulin resistance in both genotypes reflected by increased glycemia, insulinemia, and glucose-insulin product (Table 3). In wild-type mice, HFD caused a diabetic GTT (FIG. 3c). In AG4OX mice, HFD worsened glucose tolerance, but the glucose concentration curve still remained much lower than in HFD-fed wild-type mice. The enhanced glucose tolerance in AG4OX mice did not result from alterations in serum non-esterified fatty acids, triglycerides, leptin, or adiponectin levels. AG4OX mice had higher non-esterified fatty acids and triglycerides, and lower leptin levels on both diets (Table 3), and lower adiponectin levels²⁶ on chow compared to wild-type mice. This pattern is thought to contribute to insulin resistance^{2,3,27} and not to enhanced insulin sensitivity.

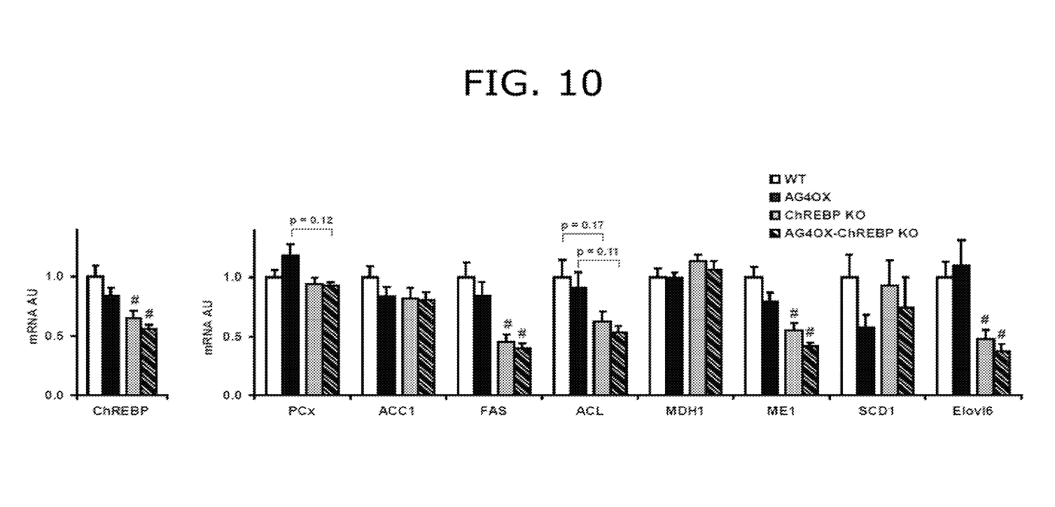

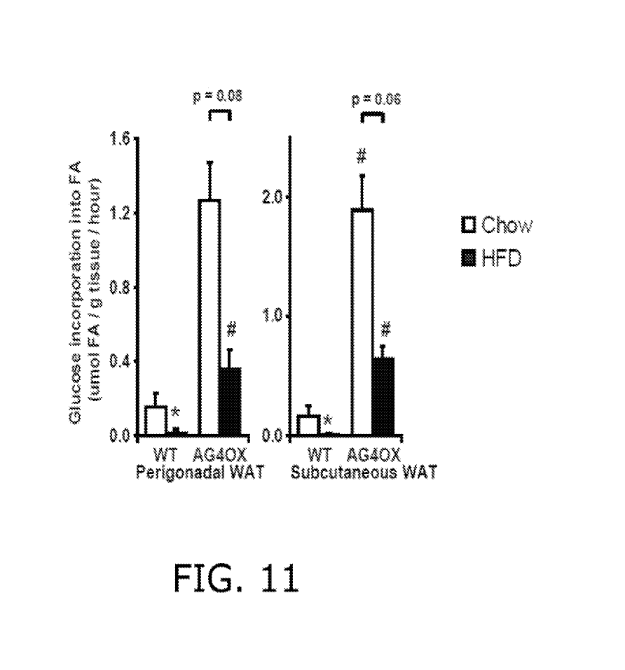

HFD reduces the expression of adipose tissue fatty acid synthetic enzymes in rodents^{28,29} and recent data from studies in humans show that downregulation of fatty-acid synthetic enzymes in adipose tissue correlates with insulin resistance⁷⁻¹⁰. Therefore, we examined whether downregulation of adipose tissue ChREBP and fatty acid synthesis may contribute to the insulin resistance resulting from HFD. HFD in wild-type mice reduced fatty acid synthetic rates in subcutaneous (78%) adipose tissue, but not perigonadal fat (FIG. 3d). In AG4OX, HFD markedly reduced fatty acid synthesis in perigonadal (63%) and subcutaneous (74%) adipose tissue, but the rates remained 2- to 7-fold higher in AG4OX compared to wild-type on HFD. This intermediate rate of fatty acid synthesis paralleled the change in glucose tolerance in AG4OX mice on HFD (FIG. 3c). Changes in glucose incorporation into newly synthesized fatty acids (FIG. 11) were similar to the changes in total (from all substrates) fatty acid synthesis (FIG. 3d) in adipose tissue in both genotypes. In the liver, fatty acid synthesis was not higher in AG4OX on either diet (FIG. 3d) suggesting that the increase in fatty acid synthesis in adipose tissue resulted from increased adipose tissue glucose flux and was not secondary to systemic effects of adipose-specific Glut4 overexpression. HFD inhibited adipose fatty acid synthesis independently of adipose tissue glucose uptake in AG4OX mice since Glut4 protein (FIG. 3e) and basal- and insulin-stimulated glucose transport remained elevated³⁰.

To investigate the molecular mechanisms responsible for the HFD-induced changes in adipose tissue fatty acid synthesis, we examined the expression of lipogenic transcription factors and fatty acid synthetic enzymes. In wild-type mice, HFD diminished ChREBP but not SREBP-1c expression in white adipose tissue (WAT) (FIG. 3f) consistent with recent reports^{29,31}. In AG4OX WAT, HFD markedly decreased ChREBP and also SREBP-1c expression but ChREBP remained elevated compared to wild-type mice on HFD. Nevertheless, HFD reduced the degree of ChREBP induction in AG4OX adipose tissue despite the fact that Glut4 overexpression persisted (FIG. 3e). Thus, HFD may regulate ChREBP expression in part by mechanisms independent of Glut4 expression and glucose flux consistent with the effects of HFD on fatty acid synthesis (FIG. 3d).

Many of the fatty acid synthetic genes that were up-regulated in WAT of AG4OX on chow are down-regulated in HFD WT and AG4OX mice (FIG. 3g) but remain higher in AG4OX WAT compared to WT in a pattern that paralleled the changes in ChREBP expression (FIG. 3f) and fatty acid synthesis (FIG. 3d). In WT WAT, the down-regulation of ChREBP most likely accounted for the HFD-induced down-regulation of fatty-acid synthetic gene expression since WAT SREBP-1c expression is not altered. Furthermore, whole body genetic ablation of SREBP-1c does not diminish the expression of these genes in adipose tissue³².

In contrast, both ChREBP and SREBP-1c expression were down-regulated in WAT in AG4OX mice on HFD compared to AG4OX on chow (FIG. 3f). The persistent modest elevation of ChREBP in WAT of AG4OX on HFD compared to WT on HFD (FIG. 3f) was likely responsible for the increased lipogenesis which contributes to the improved glucose tolerance in AG4OX (FIG. 3c).

LXRs were unlikely to contribute to the down-regulation of ChREBP expression, fatty-acid synthesis, or lipogenic enzyme expression in WT mice on HFD because expression of LXRs (FIG. 3h) and canonical LXR targets (FIG. 3i) were either unchanged or modestly increased. Mlx (Max like protein X) is an obligate dimerization partner for ChREBP transcriptional activity³³. Neither HFD nor overexpression of Glut4 altered Mlx expression in WAT (FIG. 3h).

In the liver, neither adipose-specific Glut4 overexpression nor HFD affected fatty acid synthesis compared to WT-chow (FIG. 3d), although, HFD in WT mice tended to increase SREBP1c, PCx, FAS, ACL, and MEl mRNA expression in liver (FIG. 12).

We next sought to determine whether adipose tissue ChREBP might contribute to regulating insulin sensitivity and glucose homeostasis in humans. In 123 non-diabetic individuals with normal glucose tolerance and widely ranging body mass index (20.8-50.9 kg/m²; Table 4), ChREBP expression in subcutaneous adipose tissue correlated strongly with insulin sensitivity measured by glucose infusion rate during a euglycemic-hyperinsulinemic clamp procedure (R=0.539, P<0.001; FIG. 3j). Expression of adipose tissue ChREBP correlated with Glut4 (FIG. 3k), consistent with a role for ChREBP in mediating the beneficial effects of adipose tissue Glut4 expression on glucose homeostasis.

Most, but not all, obese people are insulin resistant. To determine whether adipose tissue-ChREBP expression could have a role in regulating insulin sensitivity in obese people, we also investigated adipose tissue-ChREBP expression in a group of non-diabetic obese individuals (BMI=36.6±4.6 kg/m²) with widely ranging insulin-sensitivity (Table 4). Adipose tissue-ChREBP expression was directly associated with insulin-stimulated glucose uptake during a hyperinsulinemic-euglycemic clamp procedure, independent of BMI (R=0.605; P<0.001; FIG. 3l). Surprisingly, ChREBP expression did not correlate with Glut4 expression in these obese individuals (FIG. 3m). These results suggest that adipose tissue-ChREBP expression has beneficial effects on insulin sensitivity, even among obese subjects, and this can be independent of Glut4 gene expression.

### Identification of a Novel ChREBP Isoform

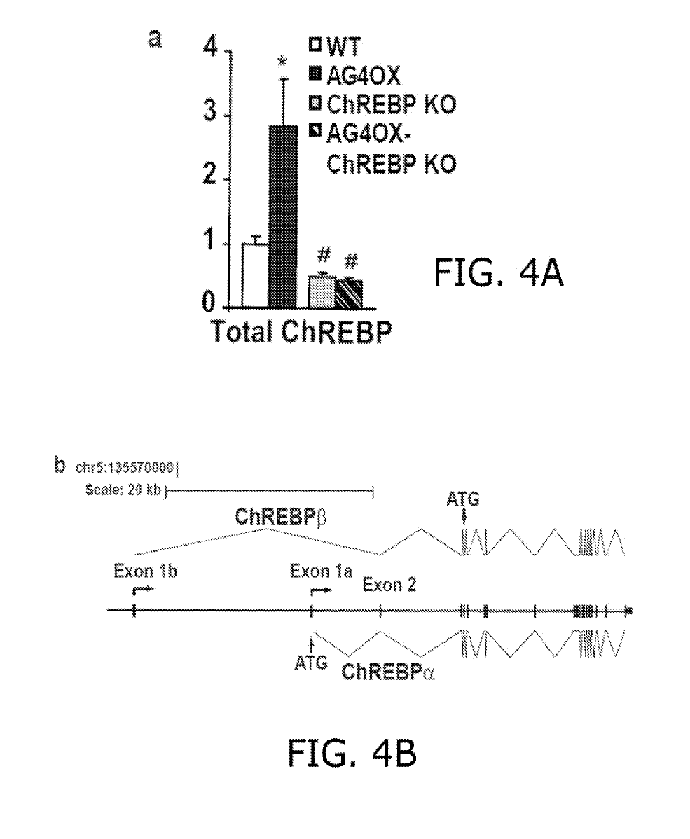

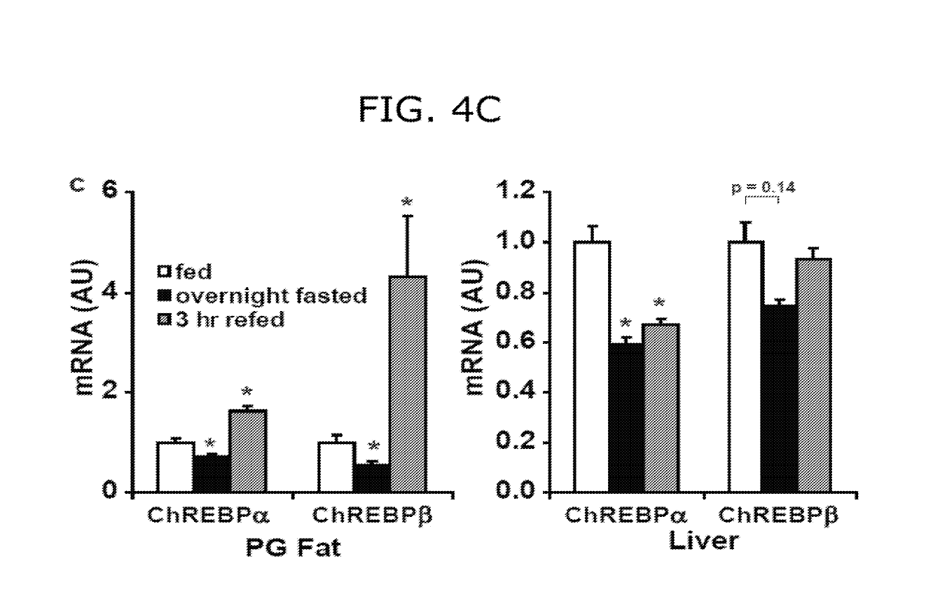

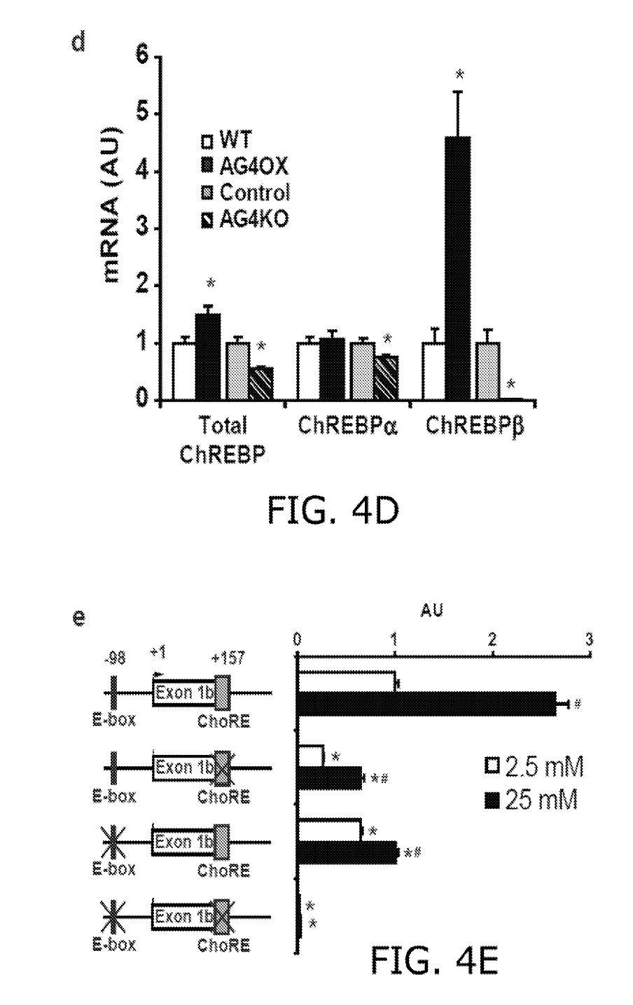

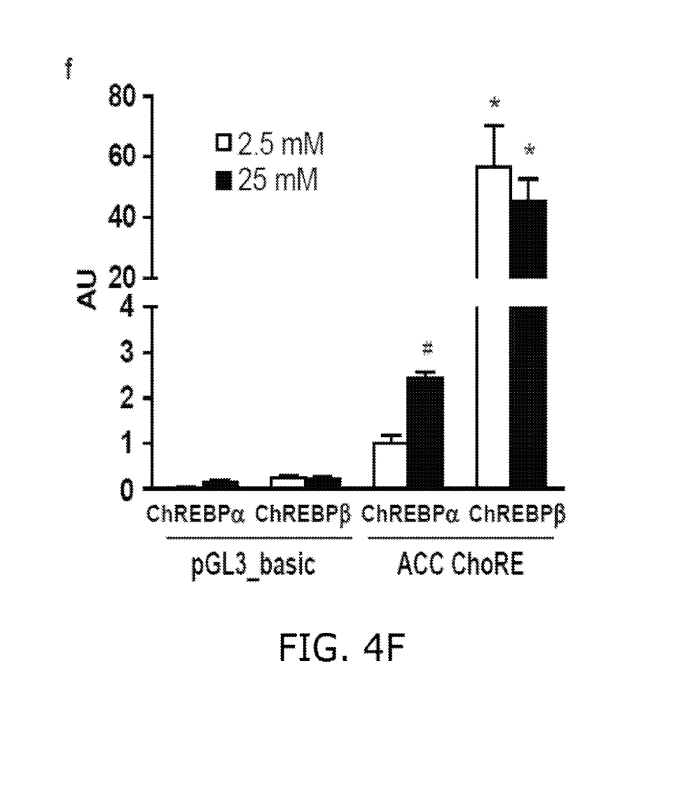

The mechanism by which glucose activates ChREBP transcriptional activity is complex and controversial. It has been attributed to xylulose-5-phosphate induced dephosphorylation of specific ChREBP residues³⁴. Alternatively, glucose or a glucose metabolite may prevent the interaction of an N-terminal inhibitory domain with a glucose-activating domain within the ChREBP protein³⁵. Glucose and other carbohydrates can also induce ChREBP expression³¹ but whether this contributes to ChREBP activity is unknown. The mechanism for carbohydrate-mediated ChREBP induction may involve “feed-forward” auto-regulation since a dominant negative mutant of Mlx which inhibits ChREBP activity reduces endogenous ChREBP expression^{21,36}. To determine whether this occurs in vivo, we used qPCR primers proximal to the deleted exons to detect ChREBP mRNA. ChREBP was downregulated in adipose tissue (FIG. 4a) and liver (FIG. 10) of ChREBP KO mice consistent with ChREBP regulating its own expression in vivo either directly or indirectly.

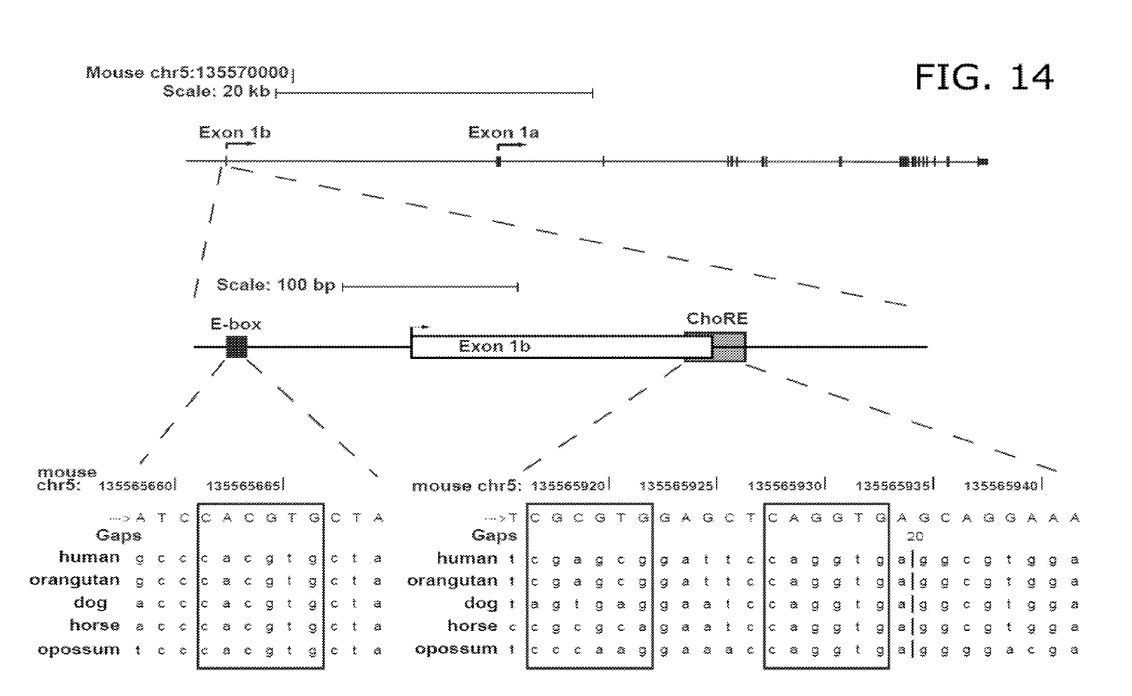

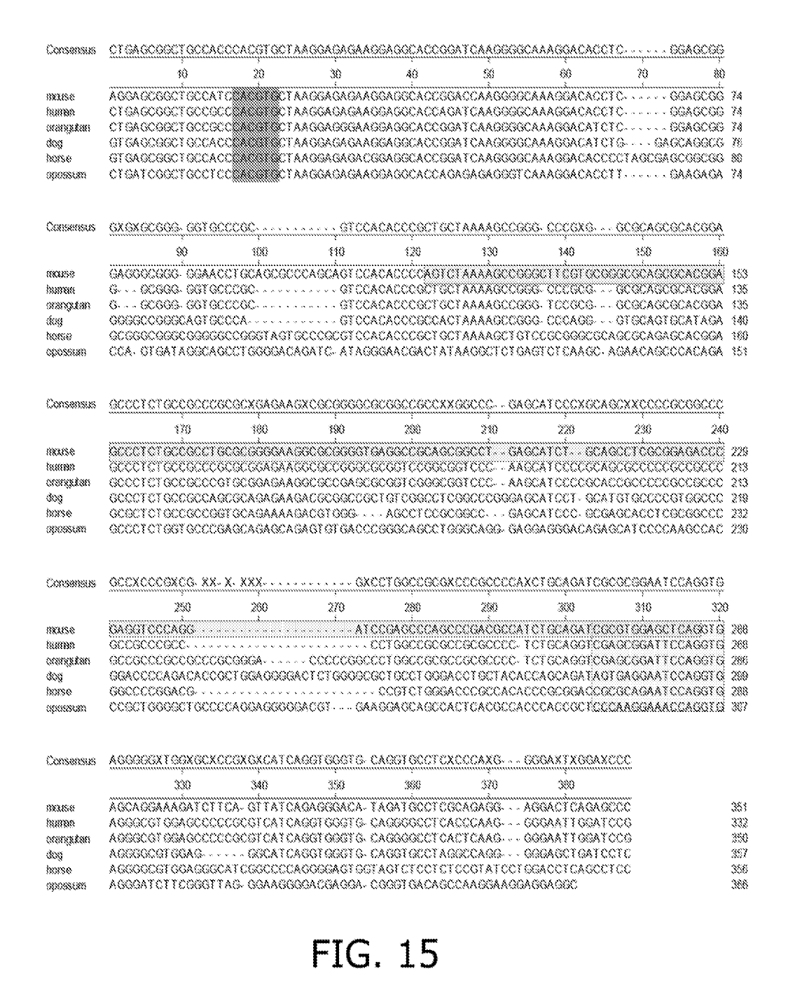

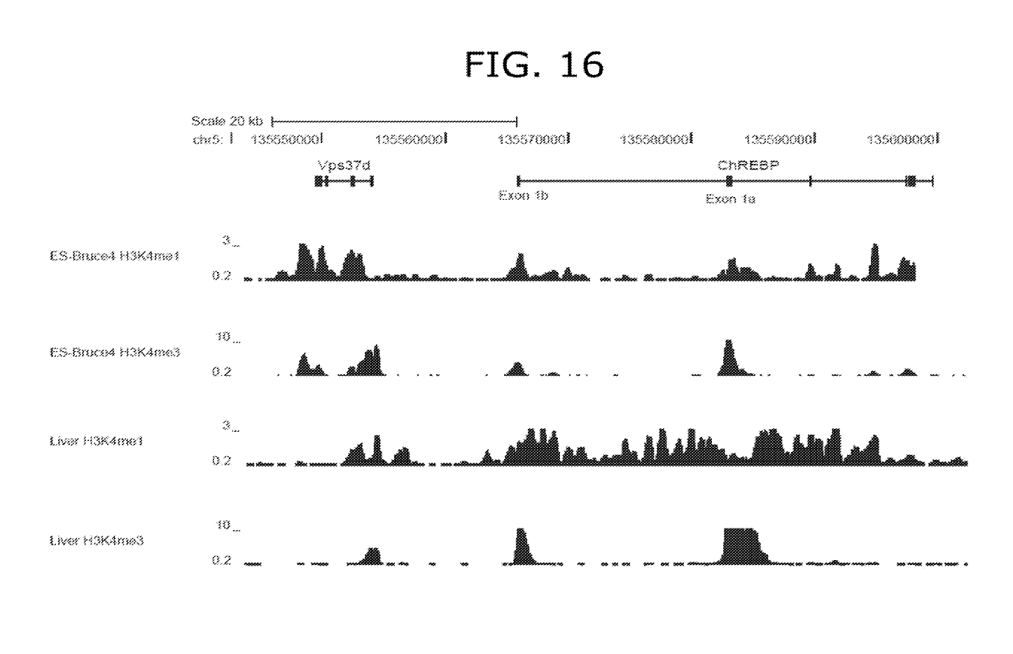

To determine the molecular mechanisms by which ChREBP transactivates its own expression, we searched the genomic sequence up- and downstream of the ChREBP transcriptional start site for a carbohydrate response element (ChoRE). The ChoRE is defined by two E-boxes (CACGTG) separated by 5 nucleotides³⁷. Using the Transcription Element Search System and a positional weight matrix defined by known ChoREs (FIG. 13), we identified a single candidate ChoRE present 17 kb upstream of the mouse ChREBP transcriptional start site (FIG. 14). Additionally, a separate E-box was identified 255 base pairs proximal to the putative ChoRE (FIG. 14). In this region, the genomic sequence including the ChoRE and 5′ E-box was found to be highly conserved (FIGS. 14 and 15). H3K4me3 and H3K4me1 histone methylation marks aligned with this conserved genomic region (FIG. 16) suggesting the presence of an alternative promoter³⁸ and potentially an alternative first exon. By 5′ rapid amplification of cDNA ends, we confirmed the presence of an alternative first exon, exon 1b (FIG. 4b and FIGS. 14-16) and cloned an mRNA species transcribed from this alternative promoter in which exon 1b is spliced to exon 2 bypassing exon 1a and retaining the remainder of exons present in the canonical ChREBP mRNA species (FIG. 4b).

### Physiologic Regulation of ChREBP Isoforms

To determine whether the two distinct ChREBP mRNA species are physiologically regulated, we performed qPCR with primers specific for ChREBPα defined by the presence of exon 1a and ChREBPβ defined by the presence of exon 1b. In adipose tissue, ChREBPα expression declined modestly with overnight fasting, and increased 64% after 3 hours of refeeding a chow diet compared to ad libitum fed mice (FIG. 4c). In contrast, adipose tissue ChREBPβ declined 45% after an overnight fast and increased 4.3-fold with refeeding compared to the ad libitum fed state. Thus, expression of ChREBPβ was more highly regulated in adipose tissue than ChREBPα with fasting and refeeding. In liver, ChREBPα declined 40% with overnight fasting and ChREBPβ expression tended to decline (25%) as well. Following 3 hours of refeeding, ChREBPα remained suppressed while ChREBPβ returns to baseline. After 6 hours of refeeding, liver ChREBPα levels had returned to normal (data not shown). Thus, in liver, the time course of regulation of ChREBPα and ChREBPβ differ. Furthermore, the refeeding effects on ChREBPβ were more rapid in adipose tissue than liver.

To determine whether alterations in glucose flux selectively regulate ChREBPα or β, we examined the expression of the two isoforms in AG4OX and AG4KO adipose tissue. Total ChREBP expression increased 50% in AG4OX and decreased 50% in AG4KO adipose tissue (FIG. 4d). The expression of ChREBPα remained unchanged in AG4OX adipose tissue and declined modestly (25%) in AG4KO adipose tissue. In contrast, in adipose tissue, ChREBPβ increased 4.6-fold in AG4OX mice, and decreased 97% in AG4KO (FIG. 4d). Considering the 50% increase in total ChREBP mRNA expression between WT and AG4OX mice compared to the 4.6-fold increase in ChREBPβ expression, we estimated that ChREBPβ represented ˜12.5% of all ChREBP mRNA species in WT adipose tissue from ad libitum fed mice. While the expression of both ChREBPα and β were nutritionally regulated (FIG. 4c), ChREBPβ alone responded robustly to Glut4-mediated changes in glucose flux (FIG. 4d).

### Glucose-Mediated Activation of ChREBPα Induces ChREBPβ

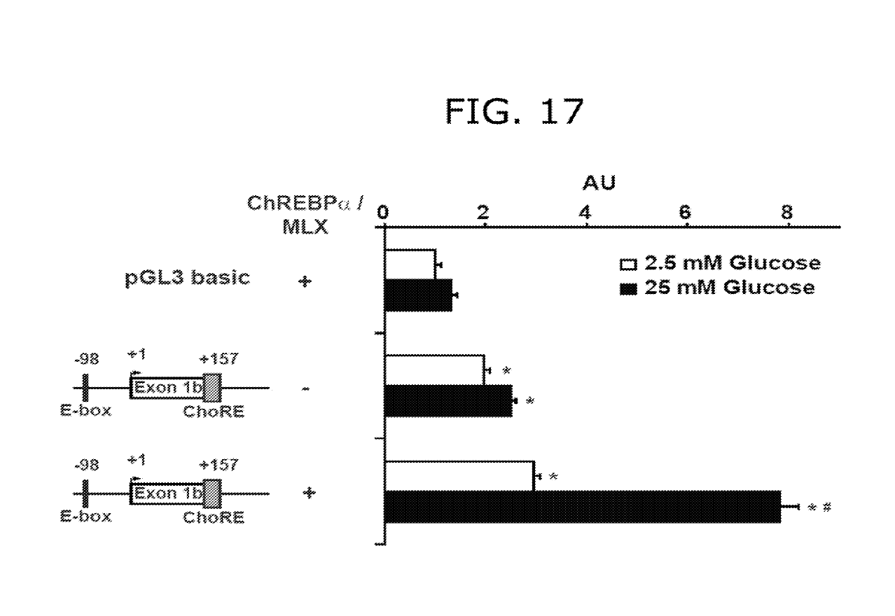

To investigate the molecular mechanism by which ChREBPβ expression is regulated in response to changes in adipose tissue Glut4 expression, we cloned the ChREBPβ promoter into a luciferase reporter plasmid. Expression of the ChREBPβ-promoter-luciferase construct was markedly increased in a glucose-dependent manner with co-transfection of ChREBPα and Mlx (FIG. 4e). Mlx, the dimerization partner for ChREBP, is required for ChREBP transcriptional activity³³. Expression of the ChREBPβ-promoter-luciferase construct in high glucose did not increase without co-transfection of ChREBPα and Mlx (FIG. 17) indicating that ChREBPβ required transactivation by ChREBPα for expression. Basal and glucose-stimulated expression was attenuated with deletion of either the ChoRE or the upstream E-box and abolished entirely with deletion of both (FIG. 4e). Glucose and ChREBPα/Mlx had no effect on the expression of a luciferase reporter containing 5 kb of the ChREBPα promoter indicating that ChREBPα does not regulate its own expression (data not shown). Thus, ChREBPα specifically regulates expression of ChREBPβ.

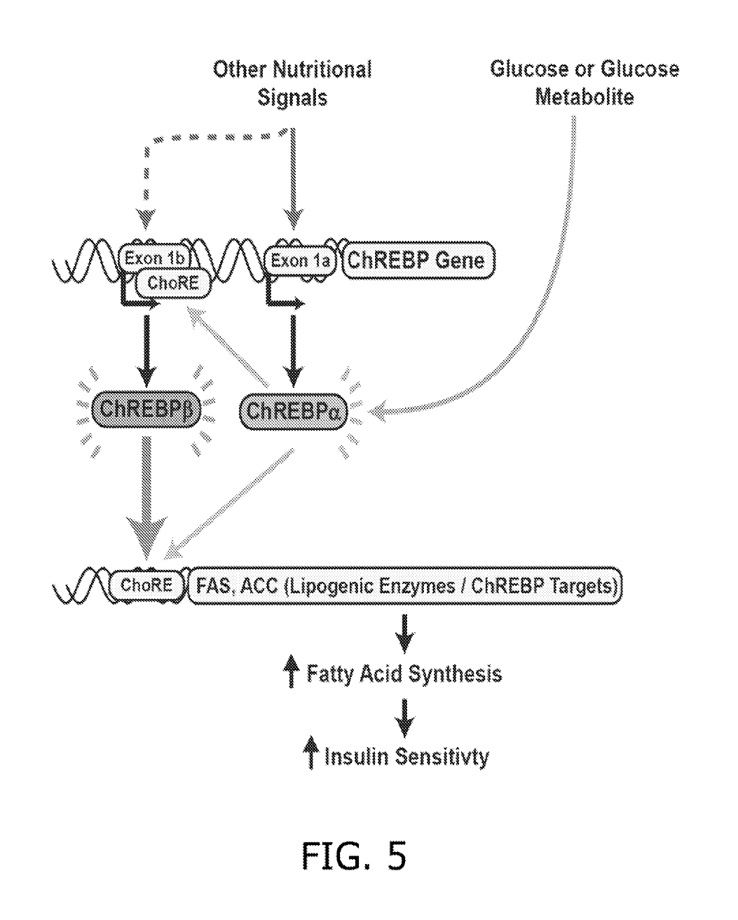

The translational start site for ChREBPα resides in exon 1a and translation from this site produces an 864 amino acid protein. No translational start site was present in exon 1b and translation beginning at the next start site, in exon 4, produces a 687 amino acid protein in which two nuclear export signals, a nuclear localization signal, and a domain that inhibits ChREBP transcriptional activity in low glucose conditions are deleted (FIG. 18)^{35,39}. ChREBP mutants with deletions of this N-terminal region demonstrate increased nuclear localization and enhanced transcriptional activity in both low and high glucose³⁵. Using an ACC-ChoRE-luciferase reporter construct⁴⁰, we compared the transcriptional activity of ChREBPα and ChREBPβ (FIG. 4f). High glucose increased ChREBPα transcriptional activity 2.4-fold compared to low glucose. ChREBPβ activity in either low or high glucose was increased ˜20-fold compared to ChREBPα activity in high glucose, but there was no glucose regulation of ChREBPβ transcriptional activity. The elevated transcriptional activity of ChREBPβ may have been due to the absence of the N-terminal inhibitory domain. Together, these results suggest the presence of a feed-forward mechanism in which glucose-mediated activation of ChREBPα transactivates the expression of ChREBPβ (FIG. 4e and FIG. 5), a more potent activator of ChREBP transcriptional targets (FIG. 4f).

### ChREBP Isoform Regulation in Pathophysiologic States

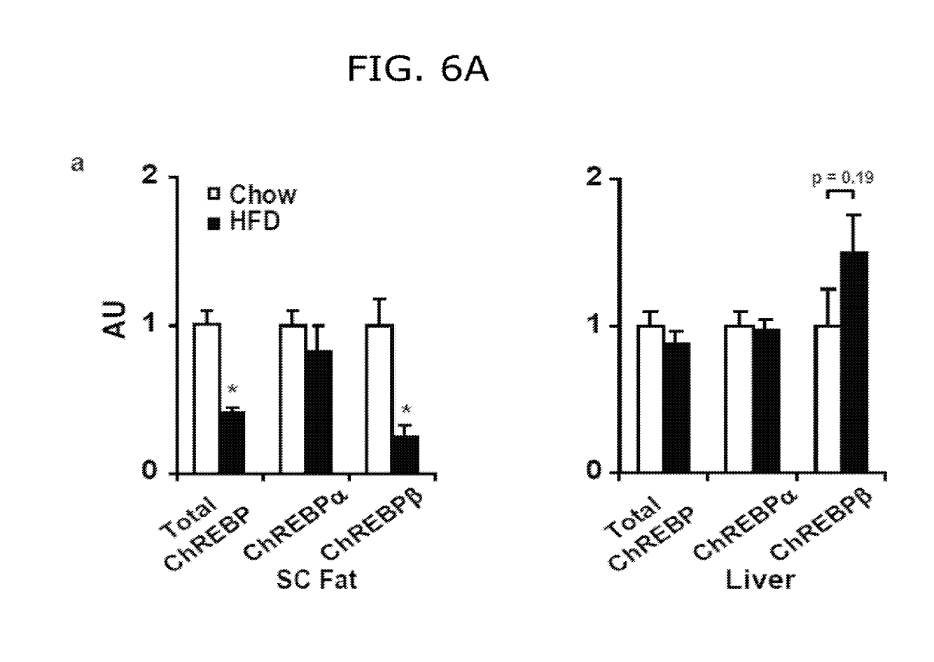

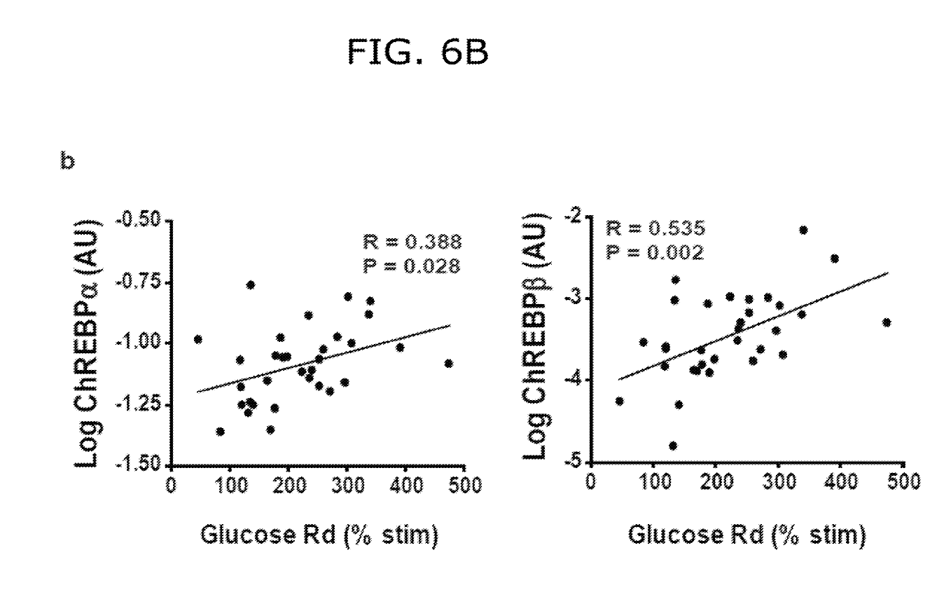

To determine whether regulation of distinct ChREBP isoforms might contribute to pathophysiology, we examined the regulation of ChREBPα and β in mice subjected to HFD. On HFD, total ChREBP and ChREBPβ expression declined in adipose tissue while ChREBPα expression remained unchanged (FIG. 6a). These results are consistent with the possibility that downregulation of adipose ChREBPβ contributes to insulin resistance. Interestingly, in liver, HFD did not alter total ChREBP, ChREBPα, or ChREBPβ expression (FIG. 6a).

To determine whether adipose tissue ChREBPα or β contributed more to insulin sensitivity in humans, we re-examined isoform-specific ChREBP expression in non-diabetic obese subjects. Expression of both isoforms correlated with insulin sensitivity (FIG. 6b). Multiple linear regression analysis demonstrated that expression of ChREBPβ (β=0.454, P=0.016) and not ChREBPα (β=0.136, P=0.356) was predictive of insulin sensitivity (Table 5).

### Significance of Adipose-ChREBP Isoform Regulation

In the past decade, the adipocyte has been recognized as a key regulator of whole-body energy homeostasis and metabolic function^{2,4}. Our prior work demonstrating that adipose tissue Glut4 expression regulates systemic insulin sensitivity^{4,5} indicated that adipocytes are capable of sensing and coordinating responses to changes in glucose availability. The elucidation here of the molecular mechanism by which glucose-mediated ChREBP activation transactivates expression of a novel, potent ChREBPβ (FIG. 5) isoform sheds new light on the complexity underlying cellular responses to changes in nutrient availability. Our data demonstrate that adipose tissue ChREBP plays a key role in integrating adipocyte and whole-body metabolic function and this may be mediated by transcriptional regulation of the potent ChREBPβ isoform.

The beneficial effects of adipose tissue-ChREBP on glucose homeostasis may result from upregulation of adipose tissue-fatty acid synthesis and/or other ChREBP-dependent actions such as changing the metabolic fate of glucose after it enters adipocytes and/or regulating a novel adipokine. The strong, direct correlation between adipose-ChREBP expression and insulin sensitivity in human subjects suggests that adipose tissue ChREBP may be involved in regulating whole-body insulin action in people. The beneficial metabolic effects of increased fatty acid synthesis in adipose tissue contrast with the adverse effects of increased fatty acid synthesis in the liver. The latter increases intrahepatic triglyceride content and may contribute to insulin resistance and other features of the metabolic syndrome^{41,42}. In summary, our data support the importance of adipose-ChREBP in regulating insulin action and glucose homeostasis in human obesity and diabetes. These data suggest that selective activation of adipose tissue ChREBP, and specifically ChREBPβ, could be an effective therapeutic strategy for preventing and treating type 2 diabetes and obesity-related metabolic diseases.

TABLE 1A

| | | | NOM | FDR |
|---|---|---|---|---|
| NAME | SIZE | NES | p-val | q-val |
| FATTY_ACID_SYNTHESIS | 8 | −1.92 | 0.00 | 0.15 |
| MALATEXPATHWAY | 6 | −1.80 | 0.00 | 0.25 |
| MATRIX_METALLOPROTEINASES | 19 | −1.76 | 0.08 | 0.24 |
| CYTOKINEPATHWAY | 22 | −1.70 | 0.03 | 0.25 |
| ST_INTERLEUKIN_13_PATHWAY | 8 | −1.67 | 0.08 | 0.25 |
| ST_IL_13_PATHWAY | 8 | −1.56 | 0.07 | 0.43 |
| ROS | 9 | −1.52 | 0.08 | 0.47 |
| MAP00480_GLUTATHIONE_METABOLISM | 26 | −1.48 | 0.10 | 0.53 |
| KREBS-TCA_CYCLE | 26 | −1.46 | 0.00 | 0.51 |
| GPCRS_CLASS_B_SECRETIN-LIKE | 16 | −1.43 | 0.10 | 0.55 |

TABLE 1B

| | | | RANK IN |
|---|---|---|---|
| PROBE | GENE SYMBOL | GENE NAME | GENE LIST |
| 93308_s_at | PCX | pyruvate carboxylase | 12245 |
| 98575_at | FASN | fatty acid synthase | 12140 |
| 162288_f_at | PCX | pyruvate carboxylase | 12044 |
| 160207_at | ACLY | ATP citrate lyase | 12038 |
| 100593_at | TNNT2 | troponin T2, cardiac | 11794 |
| 162077_f_at | SCD5 | stearoyl-CoA desaturase 5 | 4857 |
| 95758_at | SCD5 | stearoyl-CoA desaturase 5 | 4219 |
| 95485_at | HADH | hydroxyacyl-Coenzyme A | 3839 |
| | | dehydrogenase | |

### Table 1. Gene Set Enrichment Analysis (GSEA) in Adipose Tissue of AG4OX Mice.

Total RNA from epididymal adipose tissue was extracted using the RNeasy Mini Kit from Qiagen from three mice from each of two genotypes: AG4OX and FVB littermate controls. RNA from each mouse was hybridized on an Affymetrix MG-U74-A.v2 Genechip microarray containing (12,421 probes). Genome-wide expression analysis of the microarray data was performed using gene set enrichment analysis (GSEA) software version 2.05 available from the Broad Institute (http://www.broadinstitute.org/gsea/downloads.jsp)11 and gene set database version s2.mgu74av2.gmt containing 522 distinct gene sets.

In Table 1A, the top ten up-regulated gene sets ranked by “NES”—normalized enrichment score—in AG4OX adipose tissue compared to controls are listed. “Name” indicates the name of the gene set as listed in the s2.mgu74av2.gmt database. “Size” refers to the number of genes in the gene set. “Nom p-val” and “FDR q-val” indicate the nominal p-values and false discovery rates for the individual sets, respectively, as calculated by the GSEA analysis software.

In Table 1B, the Affymetrix Probe number, Gene Symbol, and Gene Name are listed for each gene included in the fatty acid synthesis gene set according to the s2.mgu74av2.gmt database. ‘Rank in Gene List’ refers to the position of the gene in the ranked list of all genes present in our expression dataset and ranked by signal to noise ratio. The GSEA algorithm calculates an enrichment score reflecting the degree to which the genes included in a gene set are overrepresented at the top or bottom of the ranked list of all genes present in the expression dataset.

TABLE 2
Serum metabolites in AG4OX mice crossbred with ChREBP KO
mice on chow diet. Serum was collected from 6-month-old female
mice in the fed state at 9 AM. Values are means ± SE. n = 9-10 per
group. Statistical comparisons were performed by ANOVA with
pairwise comparisons by Tukey's test. * p < 0.05 comparison between
different AG4OX genotypes with same ChREBP genotype; # p
< 0.05 comparison between different ChREBP genotypes with
same AG4OX genotype. † P = 0.15 compared to AG4OX; ‡ P = 0.12
compared to WT; § P = 0.20 compared to WT;
£ P = 0.07 compared to WT.

| | | | | AG4OX- |
|---|---|---|---|---|
| | WT | AG4OX | ChREBP KO | ChREBP KO |
| Glucose | 193 ± 9 | 148 ± 6 * | 211 ± 9 | 205 ± 7 # |
| (mg/dl) | | | | |
| Insulin (ng/ml) | 1.02 ± .13 | 0.82 ± 0.10 | 1.46 ± 0.21 | 1.26 ± 0.19 † |
| Glucose * | 196 ± 24 | 124 ± 17 ‡ | 314 ± 51 § | 261 ± 42 # |
| Insulin Product | | | | |
| Leptin (ng/ml) | 4.37 ± 1.17 | 2.82 ± 0.78 | 4.30 ± 1.14 | 2.35 ± 0.36 |
| NEFA (mM) | 0.66 ± 0.05 | 0.77 ± 0.05 | 0.56 ± 0.04 | 0.59 ± 0.03 # |
| Triglyceride | 62.9 ± 8.06 | 91.4 ± 10.73 £ | 50.2 ± 6.42 | 52.9 ± 5.47 # |
| (mg/dl) | | | | |

TABLE 3
Fed serum metabolites in WT vs AG4OX mice on chow vs HFD.
Serum was collected from 4-month-old male mice in the fed
state from 9 AM to 11 AM. Values are means ± SE. n = 11-17
per group. Statistical comparisons were performed by ANOVA
with pairwise comparisons by Tukey's test. * p < 0.05
comparison within genotype (chow compared to HFD); # p < 0.05
comparison within diet (WT compared to AG4OX); † p = 0.054
compared to WT-chow.

| | | | | AG4OX- |
|---|---|---|---|---|
| | WT-Chow | WT-HFD | AG4OX-Chow | HFD |
| Glucose | 225 ± 7 | 253 ± 7 * | 159 ± 6 # | 215 ± 11 *# |
| (mg/dl) | | | | |
| Insulin | 1.65 ± 0.12 | 2.96 ± 0.28 * | 1.14 ± 0.17 † | 3.27 ± 0.56 * |
| (ng/ml) | | | | |
| Glucose * | 376 ± 28 | 682 ± 75 * | 248 ± 51 # | 755 ± 169 * |
| Insulin | | | | |
| Product | | | | |
| Leptin | 4.86 ± 1.17 | 13.01 ± 0.78 * | 3.41 ± 1.14 | 5.73 ± 0.36 *# |
| (ng/ml) | | | | |
| NEFA (mM) | 0.77 ± 0.03 | 0.77 ± 0.04 | 1.14 ± 0.05 # | 0.98 ± 0.06 *# |
| Triglyceride | 196 ± 12 | 137 ± 11 * | 242 ± 13 # | 190 ± 11 *# |
| (mg/dl) | | | | |

TABLE 4
Anthropometric characteristics of human study subjects. All values
are Means ± SD
Anthropometric characteristics of cross-sectional study subjects

| | |
|---|---|
| Number (men/women) | 123 (64/59) |
| Age (years) | 57 ± 15 |
| BMI (kg/m2) | 29.8 ± 6.5 |
| % BodyFat | 31 ± 10 |

Anthropometric characteristics of obese case-control subjects

| | |
|---|---|
| Number (men/women) | 38 (28/10) |
| Age (years) | 41 ± 11 |
| BMI (kg/m2) | 36.6 ± 4.6 |
| % BodyFat | 41 ± 9 |

TABLE 5
Table 5. Linear regression analysis for ChREBPα and ChREBPβ
as predictors of insulin sensitivity as measured by insulin stimulated
glucose uptake (% over basal) in 32 obese non-diabetic individuals.
Model Summary^{b}

| | | | Std. Error of the |
|---|---|---|---|
| R | R Square | Adjusted R Square | Estimate |
| .554^{a} | .307 | .259 | 80.905 |

^{a}Predictors: (Constant), Log AT ChREBP Beta (AU), Log AT ChReBP Alpha (AU)
^{b}Dependent Variable: Glucose Rd % stim

ANOVA^{b}

| | | | Mean | | |
|---|---|---|---|---|---|
| Model | Sum of Squares | Df | Square | F | Sig. |
| Regression | 84139.554 | 2 | 42069.777 | 6.427 | .005^{a} |
| Residual | 189821.946 | 29 | 6545.584 | | |
| Total | 273961.500 | 31 | | | |

^{a}Predictors: (Constant), Log AT ChREBP Beta (AU), Log AT ChREBP Alpha (AU)
^{b}Dependent Variable: Glucose Rd % stim

Coefficients^{a}

| | Unstandardized | | | | | | | |
|---|---|---|---|---|---|---|---|---|
| | Coefficients | | Standardized | | | Correlations | | |
| | | Std. | Coefficients | | | Zero- | | |
| Model | B | Error | Beta | t | Sig. | order | Partial | Part |
| (Constant) | 609.126 | 116.106 | | 5.246 | .000 | | | |
| Log AT | 102.333 | 109.030 | .166 | .939 | .356 | .388 | .172 | .145 |
| ChREBP | | | | | | | | |
| Alpha (AU) | | | | | | | | |
| Log AT | 80.457 | 31.422 | .454 | 2.560 | .016 | .535 | .429 | .396 |
| ChREBP | | | | | | | | |
| Beta (AU) | | | | | | | | |

^{a}Dependent variable: Glucose Rd % stim

TABLE 6
Gene names, symbols, abbreviations and qPCR primer sequences,
SEQ ID NOs: 28-65, from upper left, to lower right, English reading.

| | | | | |
|---|---|---|---|---|
| | | Abbreviation | | |
| | Gene | in Text and | | |
| Gene Name | Symbol | FIGURES | Forward Primer | Reverse Primer |

Murine

| | | | | |
|---|---|---|---|---|
| 18s ribosomal | 18s | 18s | TTGACTCAACACGGGAAACC | AGACAAATCGCTCCACCAAC |
| RNA | | | | |
| acetyl- | Acaca | ACC1 | TGTACAAGCAGTGTGGGCTG | CCACATGGCCTGGCTTGGAGGG |
| Coenzyme A | | | GCT | |
| carboxylase | | | | |
| alpha | | | | |
| ATP citrate | Acly | ACL | GCCAGCGGGAGCACATC | CTTTGCAGGTGCCACTTCATC |
| lyase | | | | |
| Mlx | Mlxipl | Total | CACTCAGGGAATACACGCCT | ATCTTGGTCTTAGGGTCTTCAGG |
| interacting | | ChREBP | AC | |
| protein-like | | ChREBPα | CGACACTCACCCACCTCTTC | TTGTTCAGCCGGATCTTGTC |
| | | ChREBPβ | TCTGCAGATCGCGTGGAG | CTTGTCCCGGCATAGCAAC |
| ELOVL | Elovl6 | Elovl6 | TCAGCAAAGCACCCGAAC | AGCGACCATGTCTTTGTAGGAG |
| family | | | | |
| member 6, | | | | |
| elongation of | | | | |
| long chain | | | | |
| fatty | | | | |
| acids | | | | |
| fatty acid | Fasn | FAS | GCTGCGGAAACTTCAGGAA | AGAGACGTGTCACTCCTGGACTT |
| synthase | | | AT | |
| nuclear | Nr1h3 | LXRα | AGGAGTGTCGACTTCGCAAA | CTCTTCTTGCCGCTTCAGTTT |
| receptor | | | | |
| subfamily 1, | | | | |
| group H, | | | | |
| member 3 | | | | |
| nuclear | Nr1h2 | LXRβ | ATAGTGGGTCACGAAGCAGC | AGGGCAACAGAGTCGGAGAC |
| receptor | | | | |
| subfamily 1, | | | | |
| group H, | | | | |
| member 2 | | | | |
| malate | Mdh1 | MDH1 | AAGGCATGGAGAGGAAGGAC | AGTTCGTATTGGCTGGGTTTC |
| dehydrogenase | | | | |
| 1, NAD | | | | |
| (soluble) | | | | |
| malic enzyme | Me1 | ME1 | ATCACTTTGGATGTGGGAACAG | CAGGAAGGCGTCATACTCAGG |
| 1, NADP(+)- | | | | |
| dependent, | | | | |
| cytosolic | | | | |
| MAX-like | Mlx | Mlx | GGAGCTCTCAGCTTGTGTCT | CACCGATCACAATCTCTCGTAGA |
| protein X | | | TCA | GT |
| pyruvate | Pcx | PCx | GGAGCTTATCCCGAACATCC | CGGAAGACGTCCATACCATTC |
| carboxylase | | | | |
| stearoyl- | Scd1 | SCD1 | CCCTGCGGATCTTCCTTATC | TGTGTTTCTGAGAACTTGTGGTG |
| Coenzyme A | | | | |
| desaturase 1 | | | | |
| sterol | Srebpf1 | SREBP1c | GGAGCCATGGATTGCACATT | GGCCCGGGAAGTCACTGT |
| regulatory | | | | |
| element | | | | |
| binding | | | | |
| transcription | | | | |
| factor 1 | | | | |
| (isoform c) | | | | |
| TATA box | Tbp | Tbp | CCCTATCACTCCTGCCACAC | ACGAAGTGCAATGGTCTTTAGG |
| binding | | | | |
| protein | | | | |

Human

| | | | | |
|---|---|---|---|---|
| Mlx | Mlxipl | ChREBPα | AGTGCTTGAGCCTGGCCTAC | TTGTTCAGGCGGATCTTGTC |
| interacting | | ChREBPβ | AGCGGATTCCAGGTGAGG | TTGTTCAGGCGGATCTTGTC |
| protein-like | | | | |

### REFERENCES

1. Shepherd, P. R. & Kahn, B. B. Glucose transporters and insulin action—implications for insulin resistance and diabetes mellitus. N Engl J Med 341, 248-57 (1999).
2. Attie, A. D. & Scherer, P. E. Adipocyte metabolism and obesity. J Lipid Res 50 Suppl, S395-9 (2009).
3. Boden, G. Role of fatty acids in the pathogenesis of insulin resistance and NIDDM. Diabetes 46, 3-10 (1997).
4. Abel, E. D. et al. Adipose-selective targeting of the GLUT4 gene impairs insulin action in muscle and liver. Nature 409, 729-33 (2001).
5. Shepherd, P. R. et al. Adipose cell hyperplasia and enhanced glucose disposal in transgenic mice overexpressing GLUT4 selectively in adipose tissue. J Biol Chem 268, 22243-6 (1993).
6. Iizuka, K., Bruick, R. K., Liang, G., Horton, J. D. & Uyeda, K. Deficiency of carbohydrate response element-binding protein (ChREBP) reduces lipogenesis as well as glycolysis. Proc Natl Acad Sci USA 101, 7281-6 (2004).
7. Roberts, R. et al. Markers of de novo lipogenesis in adipose tissue: associations with small adipocytes and insulin sensitivity in humans. Diabetologia 52, 882-90 (2009).
8. Hoffstedt, J., Forster, D. & Lofgren, P. Impaired subcutaneous adipocyte lipogenesis is associated with systemic insulin resistance and increased apolipoprotein B/AI ratio in men and women. J Intern Med 262, 131-9 (2007).
9. Kursawe, R. et al. Cellularity and adipogenic profile of the abdominal subcutaneous adipose tissue from obese adolescents: association with insulin resistance and hepatic steatosis. Diabetes 59, 2288-96 (2010).
10. Ranganathan, G. et al. The lipogenic enzymes DGAT1, FAS, and LPL in adipose tissue: effects of obesity, insulin resistance, and TZD treatment. J Lipid Res 47, 2444-50 (2006).
11. Subramanian, A. et al. Gene set enrichment analysis: a knowledge-based approach for interpreting genome-wide expression profiles. Proc Natl Acad Sci USA 102, 15545-50 (2005).
12. Horton, J. D., Goldstein, J. L. & Brown, M. S. SREBPs: activators of the complete program of cholesterol and fatty acid synthesis in the liver. J Clin Invest 109, 1125-31 (2002).
13. Brown, M. S. & Goldstein, J. L. The SREBP pathway: regulation of cholesterol metabolism by proteolysis of a membrane-bound transcription factor. Cell 89, 331-40 (1997).
14. Repa, J. J. et al. Regulation of mouse sterol regulatory element-binding protein-1c gene (SREBP-1c) by oxysterol receptors, LXRalpha and LXRbeta. Genes Dev 14, 2819-30 (2000).
15. Cha, J. Y. & Repa, J. J. The liver X receptor (LXR) and hepatic lipogenesis. The carbohydrate-response element-binding protein is a target gene of LXR. J Biol Chem 282, 743-51 (2007).
16. Denechaud, P. D., Girard, J. & Postic, C. Carbohydrate responsive element binding protein and lipid homeostasis. Curr Opin Lipidol 19, 301-6 (2008).
17. Mitro, N. et al. The nuclear receptor LXR is a glucose sensor. Nature 445, 219-23 (2007).
18. Denechaud, P. D. et al. ChREBP, but not LXRs, is required for the induction of glucose-regulated genes in mouse liver. J Clin Invest 118, 956-64 (2008).
19. Pashkov, V. et al. Regulator of G protein signaling (RGS16) inhibits hepatic fatty acid oxidation in a carbohydrate response element-binding protein (ChREBP)-dependent manner. J Biol Chem 286, 15116-25.
20. Minn, A. H., Hafele, C. & Shalev, A. Thioredoxin-interacting protein is stimulated by glucose through a carbohydrate response element and induces beta-cell apoptosis. Endocrinology 146, 2397-405 (2005).
21. Ma, L., Robinson, L. N. & Towle, H. C. ChREBP*Mlx is the principal mediator of glucose-induced gene expression in the liver. J Biol Chem 281, 28721-30 (2006).
22. Su, A. I. et al. Large-scale analysis of the human and mouse transcriptomes. Proc Natl Acad Sci USA 99, 4465-70 (2002).
23. Wu, C. et al. BioGPS: an extensible and customizable portal for querying and organizing gene annotation resources. Genome Biol 10, R130 (2009).
24. Kuriyama, H. et al. Compensatory increase in fatty acid synthesis in adipose tissue of mice with conditional deficiency of SCAP in liver. Cell Metab 1, 41-51 (2005).
25. Cao, H. et al. Identification of a lipokine, a lipid hormone linking adipose tissue to systemic metabolism. Cell 134, 933-44 (2008).
26. Carvalho, E., Kotani, K., Peroni, O. D. & Kahn, B. B. Adipose-specific overexpression of GLUT4 reverses insulin resistance and diabetes in mice lacking GLUT4 selectively in muscle. Am J Physiol Endocrinol Metab 289, E551-61 (2005).
27. Ahima, R. S. & Flier, J. S. Leptin. Annu Rev Physiol 62, 413-37 (2000).
28. Nadler, S. T. et al. The expression of adipogenic genes is decreased in obesity and diabetes mellitus. Proc Natl Acad Sci USA 97, 11371-6 (2000).
29. Caesar, R. et al. A combined transcriptomics and lipidomics analysis of subcutaneous, epididymal and mesenteric adipose tissue reveals marked functional differences. PLoS One 5, e11525 (2010).
30. Gnudi, L., Tozzo, E., Shepherd, P. R., Bliss, J. L. & Kahn, B. B. High level overexpression of glucose transporter-4 driven by an adipose-specific promoter is maintained in transgenic mice on a high fat diet, but does not prevent impaired glucose tolerance. Endocrinology 136, 995-1002 (1995).
31. Sanchez, J., Palou, A. & Pico, C. Response to carbohydrate and fat refeeding in the expression of genes involved in nutrient partitioning and metabolism: striking effects on fibroblast growth factor-21 induction. Endocrinology 150, 5341-50 (2009).
32. Shimano, H. et al. Elevated levels of SREBP-2 and cholesterol synthesis in livers of mice homozygous for a targeted disruption of the SREBP-1 gene. J Clin Invest 100, 2115-24 (1997).
33. Stoeckman, A. K., Ma, L. & Towle, H. C. Mlx is the functional heteromeric partner of the carbohydrate response element-binding protein in glucose regulation of lipogenic enzyme genes. J Biol Chem 279, 15662-9 (2004).
34. Kabashima, T., Kawaguchi, T., Wadzinski, B. E. & Uyeda, K. Xylulose 5-phosphate mediates glucose-induced lipogenesis by xylulose 5-phosphate-activated protein phosphatase in rat liver. Proc Natl Acad Sci USA 100, 5107-12 (2003).
35. Li, M. V., Chang, B., Imamura, M., Poungvarin, N. & Chan, L. Glucose-dependent transcriptional regulation by an evolutionarily conserved glucose-sensing module. Diabetes 55, 1179-89 (2006).
36. Iizuka, K., Takeda, J. & Horikawa, Y. Hepatic overexpression of dominant negative Mlx improves metabolic profile in diabetes-prone C57BL/6J mice. Biochem Biophys Res Commun 379, 499-504 (2009).
37. Shih, H. M., Liu, Z. & Towle, H. C. Two CACGTG motifs with proper spacing dictate the carbohydrate regulation of hepatic gene transcription. J Biol Chem 270, 21991-7 (1995).
38. Bernstein, B. E. et al. Methylation of histone H3 Lys 4 in coding regions of active genes. Proc Natl Acad Sci USA 99, 8695-700 (2002).
39. Fukasawa, M., Ge, Q., Wynn, R. M., Ishii, S. & Uyeda, K. Coordinate regulation/localization of the carbohydrate responsive binding protein (ChREBP) by two nuclear export signal sites: discovery of a new leucine-rich nuclear export signal site. Biochem Biophys Res Commun 391, 1166-9.
40. Tsatsos, N. G. & Towle, H. C. Glucose activation of ChREBP in hepatocytes occurs via a two-step mechanism. Biochem Biophys Res Commun 340, 449-56 (2006).
41. Fabbrini, E. et al. Intrahepatic fat, not visceral fat, is linked with metabolic complications of obesity. Proc Natl Acad Sci USA 106, 15430-5 (2009).
42. Donnelly, K. L. et al. Sources of fatty acids stored in liver and secreted via lipoproteins in patients with nonalcoholic fatty liver disease. J Clin Invest 115, 1343-51 (2005).
43. Eguchi, J. et al. Transcriptional control of adipose lipid handling by IRF4. Cell Metab 13, 249-59.
44. Kotani, K., Peroni, O. D., Minokoshi, Y., Boss, O. & Kahn, B. B. GLUT4 glucose transporter deficiency increases hepatic lipid production and peripheral lipid utilization. J Clin Invest 114, 1666-75 (2004).
45. Kloting, N. et al. Serum retinol-binding protein is more highly expressed in visceral than in subcutaneous adipose tissue and is a marker of intra-abdominal fat mass. Cell Metab 6, 79-87 (2007).
46. Hems, D. A., Rath, E. A. & Verrinder, T. R. Fatty acid synthesis in liver and adipose tissue of normal and genetically obese (ob/ob) mice during the 24-hour cycle. Biochem J 150, 167-73 (1975).
47. Kent, W. J. et al. The human genome browser at UCSC. Genome Res 12, 996-1006 (2002).
48. Fujita, P. A. et al. The UCSC Genome Browser database: update 2011. Nucleic Acids Res 39, D876-82.
49. Rosenbloom, K. R. et al. ENCODE whole-genome data in the UCSC Genome Browser. Nucleic Acids Res 38, D620-5.
50. Robertson, G. et al. Genome-wide profiles of STAT1 DNA association using chromatin immunoprecipitation and massively parallel sequencing. Nat Methods 4, 651-7 (2007).

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures) are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g. elements, including numerical ranges and exemplary embodiments) of particular embodiments and aspects of the invention including the working examples. For example, particular experimental parameters exemplified in the working examples can be adapted for use in the claimed invention piecemeal without departing from the invention. For example, for materials that are disclosed, while specific reference of each various individual and collective combinations and permutation of these compounds may not be explicitly disclosed, each is specifically contemplated and described herein. Thus, if a class of elements A, B, and C are disclosed as well as a class of elements D, E, and F and an example of a combination of elements, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus to the extent an element is described in one or more references known to the person having ordinary skill in the art, they may be excluded from the claimed invention by, inter alia, a negative proviso or disclaimer of the feature or combination of features.

The described methods may be, at least in part, embodied as computer-readable implementations that can be implemented in software, hardware, or a combination of hardware and software. Examples of hardware include computing or processing systems, such as personal computers, servers, laptops, mainframes, and micro-processors. In addition, one of ordinary skill in the art will appreciate that the records and fields shown in the figures may have additional or fewer fields, and may arrange fields differently than the figures illustrate. Any of the computer-readable implementations provided by the invention may, optionally, further comprise a step of providing a visual output to a user, such as a visual representation of, for example, sequencing results, e.g., to a physician, optionally including suitable diagnostic summary and/or treatment options or recommendations.

While this invention has been particularly shown and described with references to example embodiments thereof, it will be understood by those skilled in the art that various changes in form and details may be made therein without departing from the scope of the invention encompassed by the appended claims.

1. A method of assessing the disease state and/or treatment response of a mammalian subject for a disease or disorder selected from obesity, type 2 diabetes, impaired glucose tolerance, impaired fasting glucose, metabolic syndrome, insulin resistance, vascular disease, or cancer, comprising determining the level of a ChREBP β expression product in a biological sample isolated from the mammalian subject, wherein the level of the ChREBP β expression product is indicative of the subject's disease state and/or treatment response for the disease or disorder.

2. The method of claim 1, wherein determining the level of the ChREBP β expression product comprises determining the level of a nucleic acid ChREBP β expression product.

3. The method of claim 2, wherein the level of the nucleic acid ChREBP β expression product is determined by sequencing, Northern blot, real-time-PCR, reverse-transcriptase PCR, hybridization, or microarray.

4. The method of claim 1, wherein determining the level of the ChREBP β expression product comprises determining the level of a protein ChREBP β expression product.

5. The method of claim 4, wherein the level of the protein ChREBP β expression product is determined by ELISA, Western Blot, RIA, HPLC, SPR, SAT, peptide sequencing, or MS/MS.

6. The method of claim 1, wherein the mammalian subject is a human.

7. The method of claim 1, wherein the sample is isolated from liver, adipose tissue, brown adipose tissue, muscle, pancreas, islet cells, kidney, breast, small intestine, bone marrow, nervous tissue (central, including brain or spine, and/or periperheral), prostate, ovary, cancerous, precancerous, or neoplastic tissue.

8. The method of claim 1, wherein it is determined that the subject has an abnormal level of a ChREBP β expression product, and is thereby diagnosed as having or being at increased risk for developing a disorder selected from obesity, type 2 diabetes, metabolic syndrome or insulin resistance.

9-10. (canceled)

11. A method of treating a mammalian subject having, or at increased risk for developing, a disorder selected from obesity, type 2 diabetes, metabolic syndrome, or insulin resistance, the method comprising administering to the subject an effective dose of a suitable prophylaxis or treatment, wherein the subject is assessed by the method of claim 1.

12. The method of claim 1, wherein the subject has an abnormally high level of a ChREBP β expression product, and is thereby diagnosed as having or being at increased risk for developing a disorder selected from vascular disease or cancer, preferably wherein the severity of the disorder is positively correlated to the level of the ChREBP β expression product.

13. A method of treating a mammalian subject having, or at increased risk of developing, a disorder selected from vascular disease or cancer, the method comprising administering to the subject an effective dose of a suitable prophylaxis or treatment, wherein the subject is assessed by the method of claim 1.

14-30. (canceled)

31. A screening method for identifying an agent that modulates the expression of a ChREBP β target gene comprising determining the level of a ChREBP β expression product reporter in a cell contacted with the agent, wherein a change in the level of the ChREBP β expression product reporter in the cell relative to a control cell not contacted with the candidate agent indicates that the agent modulates the expression of a ChREBP β target gene, wherein the ChREBP β expression product reporter is a synthetic ChREBP β expression product reporter.

32. The method of claim 31, wherein the agent identified is a candidate agent for treating a disorder selected from obesity, type 2 diabetes, impaired glucose tolerance, impaired fasting glucose, metabolic syndrome, insulin resistance, vascular disease or cancer.

33-40. (canceled)

41. The method of claim 31, wherein the synthetic ChREBP β expression product reporter is an expression product of a nucleic acid construct comprising a ChREBP β promoter.

42. The method of claim 41, wherein the nucleic acid construct comprises a nucleic acid sequence that has at least 60% identity to SEQ ID NO: 3 in operative association with a heterologous sequence.

43. The method of claim 42, wherein the heterologous sequence encodes a reporter molecule selected from a fluorescent protein, luciferase, aequorin, a peptide epitope, or an enzyme.

44. The method of claim 31, wherein the cell further expresses Mlx.

45. The method of claim 31, wherein the cell is contacted with glucose or fructose or one or more of their metabolites or analogs.

46. An isolated nucleic acid comprising a ChREBP β promoter sequence, wherein the ChREBP β promoter has at least 60% identity to SED ID NO: 3, and is in operative association with a heterologous sequence.

47. (canceled)
